(19) United States
(12) Patent Application Publication
(10) Pub. No.: US 2025/0127875 A1
(43) Pub. Date: Apr. 24, 2025

(54) LARGE-SCALE FLAVIVIRAL VACCINE PRODUCTION AND MANUFACTURE

(71) Applicant: Takeda Vaccines, Inc., Cambridge, MA (US)

(72) Inventors: Joseph David Santangelo, Cambridge, MA (US); Jill A. Livengood, Cambridge, MA (US); Yockann Lee, Singapore (SG); Weiwen Luo, Singapore (SG)

(21) Appl. No.: 18/832,973

(22) PCT Filed: Jan. 25, 2023
(86) PCT No.: PCT/US2023/061230

§ 371 Date: Jul. 25, 2024

Related U.S. Application Data
(60) Provisional application No. 63/385,309, filed on Nov. 29, 2022.
(60) Provisional application No. 63/385,274, filed on Nov. 29, 2022.
(60) Provisional application No. 63/302,920, filed on Jan. 25, 2022.
(60) Provisional application No. 63/302,910, filed on Jan. 25, 2022.

(51) Int. Cl. A61K 39/12; A61K 39/00; B01D 15/36; B01D 61/14; C12N 5/071; C12N 7/00

(52) U.S. Cl. CPC A61K 39/12; B01D 15/363; B01D 61/145; C12N 5/0686; C12N 7/00; A61K 2039/5254; C12N 2523/00; C12N 2770/24134; C12N 2770/24151

(57) ABSTRACT

The present invention provides methods for large-scale flaviviral vaccine production and manufacture. The methods provided herein are specifically contemplated for large-scale production and manufacture of live, attenuated flaviviral vaccines such as live, attenuated, dengue virus vaccines. Further, the methods provided herein pertain to formulation of live, attenuated, monovalent, divalent, trivalent, or tetravalent viral vaccine products.

### CROSS REFERENCE TO RELATED APPLICATIONS

The present application claims the benefit of, and priority to U.S. provisional application No. 63/302,910 filed on 25 Jan. 2022, U.S. provisional application No. 63/302,920 filed on 25 Jan. 2022, U.S. provisional application No. 63/385,274 filed on 29 Nov. 2022, and U.S. provisional application No. 63/385,309 filed on 29 Nov. 2022, the entire contents of which is incorporated herein by reference in its entirety.

### SEQUENCE LISTING

The instant application contains a Sequence Listing which is submitted herewith in electronically readable XML ST.26 format via Patent Center, and is hereby incorporated by reference in its entirety. The electronic Sequence Listing file was created on Jan. 24, 2023, is named “T08530WO.xml” and is 65 KB in size.

### FIELD OF THE INVENTION

The present invention provides methods for large-scale flaviviral vaccine production and manufacture. The methods provided herein are specifically contemplated for large-scale production and manufacture of live, attenuated flaviviral vaccines such as live, attenuated, dengue virus vaccines. Further, the methods provided herein pertain to formulation of live, attenuated, monovalent, divalent, trivalent, or tetravalent viral vaccine products.

### BACKGROUND OF THE INVENTION

Expanding vaccine manufacturing capacity is essential to stopping global transmission of diseases as well as halting the emergence of new variants of viruses. One of the biggest challenges of vaccine production and manufacture is the economical use of resources to meet the increasing delivery challenges to billions of people around the world.

One group of viruses that continue to pose an expanding threat to public health are the Flaviviruses. These are vector-borne RNA viruses that can emerge unexpectedly in human populations and cause a spectrum of potentially severe diseases including hepatitis, vascular shock syndrome, encephalitis, acute flaccid paralysis, congenital abnormalities, and fetal death. This epidemiological pattern has occurred numerous times during the last 70 years, including epidemics of dengue virus and West Nile virus, and the most recent explosive epidemic of Zika virus in the Americas. Flaviviruses are now globally distributed and infect up to 400 million people annually. Of significant concern, outbreaks of other less well-characterized flaviviruses have been reported in humans and animals in different regions of the world. The potential for these viruses to sustain epidemic transmission among humans is poorly understood. Thus, there is an urgent need for more economical, efficient, and robust methods for large-scale production and manufacture of flaviviruses that can reliably ramp up the manufacturing capacity and deliver on high demands.

Early signs of supply pressure are observed across all vaccine manufacturing steps e.g., the working virus seed that is used to infect the first production culture, or bioreactors that produce vaccine components, or chromatography and filtration steps that purify the virus, or filters and vials that comprise the vaccine components. These individual challenges can have a compound effect: the absence of a single input disrupts the entire manufacturing process. Thus, there is an urgent need for re-inventing large-scale vaccine production and manufacture at every step that provides for an economically tailored process synchronized with the increasing demand of vaccines.

### SUMMARY OF THE INVENTION

The present invention provides a method for large-scale flaviviral vaccine production and manufacture comprising the following sequential steps (i) providing cells in growth media, (ii) infecting the cells of step (i) with infection media comprising flavivirus at a low MOI, (iii) harvesting to obtain a harvest, (iv) processing the harvest of step (iii) to obtain a processed harvest, and (v) purifying the processed harvest of step (iv), wherein step (v) comprises at least one chromatography step.

The present invention provides a method for large-scale flaviviral vaccine production and manufacture comprising the following sequential steps (i) providing cells in growth media, (ii) infecting the cells of step (i) with infection media comprising flavivirus at a low MOI, (iii) harvesting to obtain a harvest, (iv) processing the harvest of step (iii) to obtain a processed harvest, and (v) purifying the processed harvest of step (iv), wherein step (v) comprises at least one chromatography step. In this context, the method of infection in step (ii) is static, and wherein the cells are infected in monolayer.

The present invention provides a method for large-scale flaviviral vaccine production and manufacture comprising the following sequential steps (i) providing cells in growth media, (ii) infecting the cells of step (i) with infection media comprising flavivirus at a low MOI, (iii) harvesting to obtain a harvest, wherein harvesting comprises a first harvesting step and at least one further harvesting step, and wherein at least 12 to 30 hours before the first harvesting step, a media change is carried out, (iv) processing the harvest of step (iii) to obtain a processed harvest, and (v) purifying the processed harvest of step (iv), wherein step (v) comprises at least one chromatography step.

The present invention provides a method for large-scale flaviviral vaccine production and manufacture comprising the following sequential steps (i) providing cells in growth media, (ii) infecting the cells of step (i) with infection media comprising flavivirus at a low MOI, (iii) harvesting to obtain a harvest, wherein harvesting comprises a first harvesting step and at least one further harvesting step, and wherein at least 12 to 30 hours before the first harvesting step, a media change is carried out, (iv) processing the harvest of step (iii) to obtain a processed harvest, and (v) purifying the processed harvest of step (iv), wherein step (v) comprises at least one chromatography step. In this context, the method of infection in step (ii) is static, and wherein the cells are infected in monolayer.

The present invention provides a method for large-scale flaviviral vaccine production and manufacture comprising the following sequential steps (i) providing cells in growth media, (ii) infecting the cells of step (i) with infection media comprising flavivirus at a low MOI, (iii) harvesting to obtain a harvest, (iv) processing the harvest of step (iii) to obtain a processed harvest, and (v) purifying the processed harvest of step (iv), wherein step (v) comprises at least one chromatography step, and wherein step (v) comprises the sequential steps of (v-a) ion exchange chromatography to obtain a purified harvest, and (v-b) ultrafiltration.

The present invention provides a method for large-scale flaviviral vaccine production and manufacture comprising the following sequential steps (i) providing cells in growth media, (ii) infecting the cells of step (i) with infection media comprising flavivirus, (iii) harvesting to obtain a harvest, (iv) processing the harvest of step (iii) to obtain a processed harvest, and (v) purifying the processed harvest of step (iv), wherein step (v) comprises at least one chromatography step, wherein the pH throughout steps (iii) to (v) is maintained at a range from 7.6 to 8.1, and wherein the difference between (a) the maximum pH occurring at any time point through out steps (iii) to (v), and (b) the minimum pH occurring at any time point through out steps (iii) to (v), is no greater than 0.4 or 0.3 units.

According to the invention, preferably, total surface area of production culture is achievable in the magnitude of 35,000 cm² or more, such as, for example, 50,000 cm² to 1,000,000 cm², 100,000 cm² to 1,000,000 cm², 50,000 cm² to 450,000 cm², 100,000 cm² to 450,000 cm².

According to the invention, preferably flavivirus is a dengue virus, preferably, the dengue virus is selected from a group consisting of live, attenuated dengue-2 virus serotype, a dengue 2/1 chimera, a dengue 2/3 chimera and a dengue 2/4 chimera.

The present invention also provides a method for large-scale flaviviral vaccine production and manufacture comprising formulation of a drug product. The drug product preferably comprises a tetravalent dengue viral vaccine comprising live, attenuated dengue-2 virus serotype, a dengue 2/1 chimera, a dengue 2/3 chimera and a dengue 2/4 chimera.

### BRIEF DESCRIPTION OF FIGURES

FIG. 1—Example overview of Flaviviral vaccine production and manufacture process. In this Figure, an example of a manufacturing process according to the invention for a tetravalent dengue viral vaccine is shown. Reference to individual serotypes as Den-1, Den-2, Den-3, or Den-4, can be construed either as non-chimeric dengue serotypes 1, 2, 3, and 4, respectively, or one or more chimeric dengue serotypes, such as e.g., four chimeric dengue serotypes or three chimeric dengue serotypes such as e.g., dengue 2/1 chimera or TDV-1 represented by SEQ ID NO: 1 and/or SEQ ID NO: 2, dengue 2 serotype or TDV-2 represented by SEQ ID NO: 3 and/or SEQ ID NO: 4, dengue 2/3 chimera or TDV-3 represented by SEQ ID NO: 5 and/or SEQ ID NO: 6 and dengue 2/4 chimera or TDV-4 represented by SEQ ID NO: 7 and/or SEQ ID NO: 8, respectively.

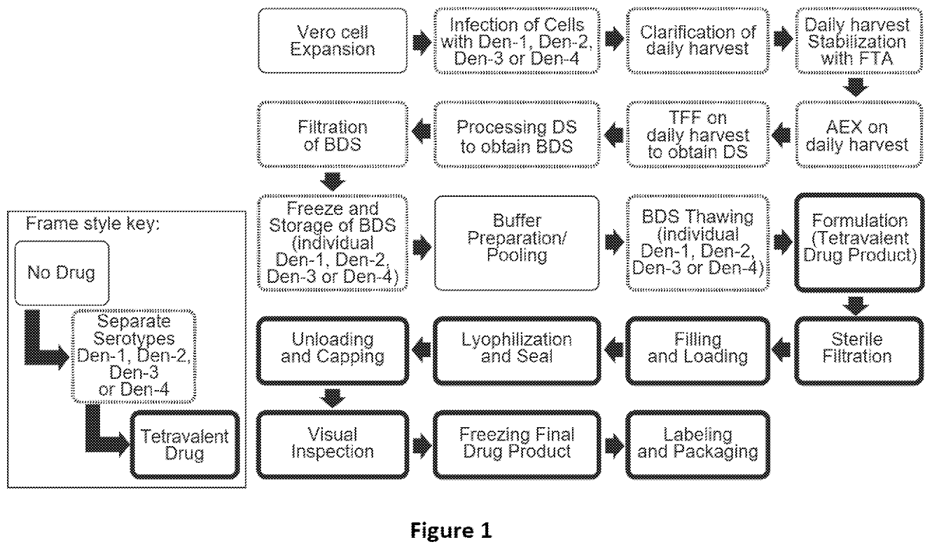

FIG. 2—Example overview of Flaviviral vaccine production and manufacture process. In this Figure, an example of a manufacturing process according to the invention for a tetravalent dengue viral vaccine is shown. In step “Processing BDS” Filtration could occur before and/or after Freeze and Storage of BDS. Reference to individual serotypes as Den-1, Den-2, Den-3, or Den-4, can be construed either as non-chimeric dengue serotypes 1, 2, 3, and 4, respectively, or one or more chimeric dengue serotypes, such as e.g., four chimeric dengue serotypes or three chimeric dengue serotypes such as e.g., dengue 2/1 chimera or TDV-1 represented by SEQ ID NO: 1 and/or SEQ ID NO: 2, dengue 2 serotype or TDV-2 represented by SEQ ID NO: 3 and/or SEQ ID NO: 4, dengue 2/3 chimera or TDV-3 represented by SEQ ID NO: 5 and/or SEQ ID NO: 6 and dengue 2/4 chimera or TDV-4 represented by SEQ ID NO: 7 and/or SEQ ID NO: 8, respectively. In one embodiment, TDV-1 and TDV-2 are manufactured in the same way. In one embodiment, TDV-3 and TDV-4 are manufactured in the same way as TDV-1 and TDV-2 but differ in the scale of manufacturing and/or the processing of the bulk drug substance. In one embodiment, TDV-4 is manufactured at a larger scale than TDV-1, TDV-2, and TDV-3. In one embodiment, TDV-3 and TDV-4 are manufactured at a larger scale than TDV-1 and TDV-2.

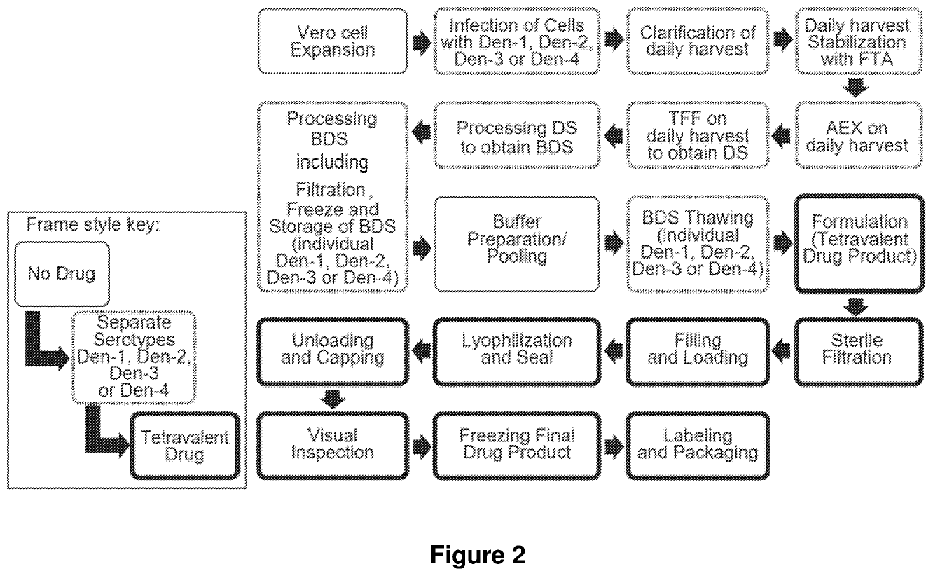

FIG. 3—Graph of viral titers obtained after infection with low MOI of 0.005 (Group 1) and high MOI of 0.05 (Group 2). Individual viral titers for days 5, 6 and 9 are shown (bars left to right).

FIG. 4—Graph of viral titers obtained after infection with low MOI of 0.001 (Group 1) and high MOI of 0.05 (Group 2). Individual viral titers for days 5, 6 and 9 are shown (bars left to right).

FIG. 5—Graph showing viral yield (grams) dependence on pH fluctuation-lower the fluctuation higher the viral yield.

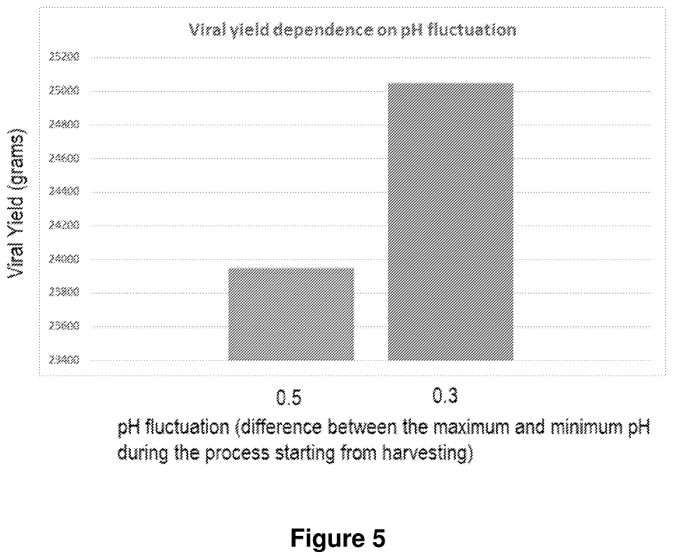

### DETAILED DESCRIPTION OF THE INVENTION

Throughout the description, references to the word “can”, (e.g. such as the phrase “can be”) are not construed as limiting the invention to specific embodiments but rather are illustrative and non-limiting examples.

Throughout the description, reference to multiple steps being carried out “sequentially” means that the steps are necessarily carried out in that order. However, when the word “sequentially” is not mentioned, the steps may or may not be carried out in that order.

The method of the invention pertains to large-scale production and manufacture of flaviviral vaccines. “Large-scale” also called “production scale” or “manufacturing scale” as used herein refers to isolation of the virus achievable from volumes of starting sample in the order of thousands of litres or thousands of square centimetres. Viewed alternatively, large-scale refers to starting production cultures of at least 1000 litres or more or at least 35,000 cm² or more. In the context of the present invention, large-scale production means a “higher” number of tissue culture vessels with a “higher” surface area for the expansion and growth of cells. For large-scale production and manufacture of flaviviral vaccines according to the present invention, culture vessels that can be used are e.g., Corning® CellSTACK® culture chambers or Nunc™ EasyFill™ Cell Factory™ systems.

For example, the Corning® CellSTACK® culture chambers that can be used are 1-stack (CF1) with 636 cm² cell growth area, 2-stack (CF2) with 1,272 cm² cell growth area, 5-stack (CF5) with 3,180 cm² cell growth area, 10-stack (CF10) with 6,360 cm² cell growth area, 40-stack (CF40) with 25,440 cm² cell growth area. The recommended medium volume required for each of these vessels is 130 to 200 mL for CF1, 260 to 400 mL for CF2, 650 to 1,000 mL for CF5, 1,300 to 2,000 mL for CF10, and 5,200 to 8,000 mL for 40-stack.

Thus, for large-scale production and manufacture of flaviviral vaccine, the “higher” number of culture vessels can be any of the combinations of the culture vessels specified above or other commercially available culture vessels with which the total volume of production culture is achievable in the magnitude of at least 1000 litres or with which the total surface area of production culture is achievable in the magnitude of 35,000 cm² or more, such as, for example, 50,000 cm² to 1,000,000 cm², 100,000 cm² to 1,000,000 cm², 50,000 cm² to 450,000 cm², 100,000 cm² to 450,000 cm².

Thus, for example, for large-scale production and manufacture of flaviviral vaccines 5 to 20 CF10 chambers, 10 to 20 CF10 chambers, 15 to 20 CF10 chambers can be used, or 1 to 20 CF40, 4 CF40, 5 to 20 CF40, 10 to 20 CF40, 15 to 20 CF40 chambers or any combination thereof can be used. It is understood that each individual possibility can be combined with another possibility to arrive at a combination of features and that each possibility, and each combination represents a separate embodiment of the invention.

A skilled person understands that a large-scale production is specifically designed to meet the demands of viral vaccine production and manufacture while economically improving the process resources. Thus, the method of the present invention specifically concerns large-scale production and manufacture of viral vaccines, the scientific, and economical considerations for which are entirely different from a small-scale process. Accordingly, the present invention preferably excludes small-scale processes of viral vaccine production.

A skilled person will also understand that the method of the present invention concerns the “production” of a flaviviral vaccine using “seed” stocks. The terms “production culture” and “seed culture” are well-known in the art. Seed culture, in general, refers to a pure culture of the desired strain, i.e., primary strain, and an inoculum refers to a seed culture to be inoculated to a medium for proliferation, which is then considered a production culture. Thus, a skilled person will understand that the methods used to develop seed stocks, or the methods used for seed culture in general are not applicable or irrelevant and cannot be used for large-scale production and manufacture of viral vaccines.

The method of the invention also pertains to large-scale production and manufacture of “attenuated” viral vaccines. The large-scale production and manufacture of attenuated viral vaccines is a challenge because attenuated viruses may have a compromised replication rate. As used herein, the term “replication”, refers to the process in which a complementary strand of a nucleic acid molecule is synthesized by a polymerase enzyme. In the context of the present invention, the term “replication” in reference to a virus refers to the completion of a flaviviral life cycle, wherein infectious viral particles or virions attach to the surface of the host cell (usually binding to a specific cell surface molecule that accounts for the specificity of the infection). Once inside the cell, the virions are uncoated and flaviviral genes begin to express leading to the synthesis of proteins needed for replication of the genome and synthesis of new proteins to make new capsids and cores leading to the assembly of progeny infectious virus particles which, themselves, are capable of infecting and replicating in new host cells. Thus, a viral life cycle is only complete if, within a single cell, infection by one or more virus particles or virions proceeds all the way to the production of fully infectious progeny virus particles. The term “replication rate” refers to the factor by which the flaviviral population grows. Thus, as used herein the term “attenuated” means that replication rate of the virus has been compromised. Several methods are known to a skilled person to assess attenuation of a particular virus. For example, attenuation of a dengue virus can be measured using methods including temperature sensitivity, small plaque size, decreased replication in mosquito C6136 cell culture, decreased replication in intact mosquitoes, and decreased incidence of viremia in monkeys.

The method of the present invention can be used for large-scale production and manufacture of flaviviruses. The genus flavivirus comprises enveloped positive-stranded RNA viruses such as West Nile (WN) virus, Japanese Encephalitis virus (JEV), Zika virus, Dengue fever virus, yellow fever virus (YF), Kyasanur Forest disease virus, Murray Valley encephalitis virus, St. Louis encephalitis virus, Tick-borne encephalitis virus, West Nile encephalitis virus, Central European encephalitis (TBE-W) virus, Far Eastern encephalitis (TBE-FE) virus, Kunjin virus, Tyuleniy virus, Ntaya virus, Uganda S virus, Modoc virus, BVDV (e.g, strains NADL, and 890), CSFV Alfort 187, BDV BD31 virus, and/or GB virus-A, -B, and/or -C.

The genus flavivirus contains highly pathogenic and potentially haemorrhagic fever viruses, such as yellow fever virus and dengue virus, Zika virus, encephalitic viruses, such as Japanese encephalitis virus, Murray Valley encephalitis virus and West Nile virus, and several less pathogenic viruses. The flavivirus genome comprises in 5′ to 3′ direction: a 5′-noncoding region (5′-NCR), a capsid protein (C) encoding region, a pre-membrane protein (prM) encoding region, an envelope protein (E) encoding region, a region encoding non-structural proteins (NS1, NS2A, NS2B, NS3, NS4A, NS4B, NS5) and a 3′ noncoding region (3′-NCR). The flaviviral structural proteins are C, prM and E, and the non-structural proteins are NS1 to NS5. The structural and non-structural proteins are translated as a single polyprotein and processed by cellular and flaviviral proteases.

Preferred flavivirus vaccines for manufacture according to the present invention are zika and dengue vaccines. Thus, the method of the present invention can be used for the large-scale production and manufacture of dengue viruses, i.e., each of the dengue serotypes. Dengue virus as used herein is a single stranded, positive sense RNA virus of the family flaviviridae. The family flaviviridae includes three genera, flavivirus, hepacivirus and pestivirus. As used herein, the term “dengue serotype” refers to a species of dengue virus which is defined by its cell surface antigens and therefore can be distinguished from other dengue serotypes by serological methods known in the art. At present, four serotypes of dengue virus are known, i.e., dengue serotype 1 (DENV-1), dengue serotype 2 (DENV-2), dengue serotype 3 (DENV-3) and dengue serotype 4 (DENV-4). Thus, the method of the present invention can also be used for large-scale production and manufacture of four live, attenuated, dengue virus strains, separately.

As used herein, the term “live, attenuated dengue virus” refers to a viable and infectious dengue virus which is mutated to provide reduced virulence. The live, attenuated dengue virus can be a dengue virus in which all components are derived from the same dengue serotype or it can be a chimeric dengue virus having parts from two or more dengue serotypes. A “virus strain” and in particular a “dengue virus strain” is a genetic subtype of a virus, in particular, of a dengue virus, which is characterized by a specific nucleic acid sequence. A dengue serotype may comprise different strains with different nucleic acid sequences, which have the same cell surface antigens and are therefore recognized by the same antibodies. A dengue virus strain can be a dengue virus in which all components are derived from the same dengue serotype or it can be a chimeric dengue virus having parts from two or more dengue serotypes.

The live, attenuated dengue virus strains can e.g., be (i) a chimeric dengue serotype 2/1 strain (TDV-1), (ii) a dengue serotype 2 strain (TDV-2), (iii) a chimeric dengue serotype 2/3 strain (TDV-3), and (iv) a chimeric dengue serotype 2/4 strain (TDV-4).

TDV-1, TDV-2, TDV-3 and TDV-4 together form a tetravalent dengue virus composition termed “TDV” or, “TAK-003”, a dengue vaccine candidate for which regulatory filings have recently been submitted by Takeda.

In one embodiment, the tetravalent dengure virus composition, “TAK-003”, comprises:
live attenuated dengue virus serotype 1,
live attenuated dengue virus serotype 2,
live attenuated dengue virus serotype 3, and
live attenuated dengue virus serotype 4;
wherein the dengue serotype 1 is a chimeric dengue serotype 2/1 strain, the dengue serotype 2 is a non-chimeric dengue serotype 2 strain, the dengue serotype 3 is a chimeric dengue serotype 2/3 strain and the dengue serotype 4 is a chimeric dengue serotype 2/4 strain;
wherein the dengue serotype 2 strain is derived from the wild type virus strain DEN-2 16681 and differs in at least three nucleotides from the wild type as follows:
a) 5′-noncoding region (NCR)-57 (nt-57)
b) NS1-53 Gly-to-Asp (nt-2579)
c) NS3-250 Glu-to-Val (nt-5270);
wherein the three chimeric dengue strains are derived from the dengue serotype 2 strain by replacing the structural proteins prM and E from serotype 2 strain with the corresponding structural proteins from the other dengue serotypes, resulting in the following chimeric dengue strains:
a DENV-2/1 chimera,
a DENV-2/3 chimera, and
a DENV-2/4 chimera.

A “chimeric virus” or “chimeric strain” or “chimeric virus strain” in general comprises parts from at least two different viruses. For example, a chimeric virus can comprise the prM and E proteins of dengue virus and the other proteins from another flavivirus. The chimeric virus can comprise the prM and E proteins of dengue virus and the other proteins from another flavivirus such as yellow fever virus, Zika virus, West Nile virus, Japanese encephalitis virus, St. Louis encephalitis virus and tick-borne encephalitis virus. the chimeric virus can comprise the prM and E proteins of dengue virus and the other proteins from yellow fever virus strain YF-17D. Such chimeric viruses are present in the commercial product Dengvaxia® and are described in, e.g., WO 98/37911, WO 03/101397, WO 2007/021672, WO 2008/007021, WO 2008/047023 and WO 2008/065315. Certain embodiments of the present invention are thus including the manufacture of tetravalent compositions of four chimeric dengue strains, and/or tetravalent compositions of four live, attenuated dengue strains, such as e.g., four live, attenuated chimeric dengue strains.

A “chimeric dengue virus” or “chimeric dengue serotype strain” or “chimeric dengue strain” preferably comprises parts from at least two different dengue serotypes, i.e., a dengue-dengue chimera. Such chimeric dengue viruses are described in WO 01/060847 A2, WO 2014/150939 A2 and WO 2017/179017 A1.

In a preferred embodiment, the live, attenuated dengue-2 virus serotype or DENV-2 or TDV-2 is in the form of DENV-2 16681 derived DEN-2 PDK-53 variant with a triple mutation at NS1-53, at 5′NCR-57 and at NS3-250 such that the amino acid position 250 of the NS3 protein contains a valine residue, and wherein the chimeras have said DEN-2 PDK-53 genome as viral backbone and one or more structural protein genes encoding capsid, premembrane/membrane or envelope of said DEN-2 PDK-53 genome or combinations thereof replaced with one or more corresponding structural protein genes from DEN-1, DEN-3 or DEN-4.

In a preferred embodiment, the dengue 2/1 chimera or DENV-2/1 chimera, or TDV-1 has two further mutations such that the amino acid position 116 of the NS2A protein contains a leucine residue and the amino acid position 92 of the NS2B protein contains an aspartic acid residue, wherein the live, attenuated dengue-2 virus serotype or DENV-2 or TDV-2 has two further mutations such that the amino acid position 52 of the prM protein contains a glutamic acid residue and the amino acid position 412 of the NS5 protein contains a valine residue, wherein the dengue-2/3 chimera or DENV-2/3 chimera, or TDV-3 has said DEN-2 PDK-53 genome as viral backbone and has the prM-E gene (nt-457 to -2373) of said DEN-2 PDK-53 genome replaced with the corresponding prM-E gene from wild-type DEN-3 16562 and wherein said dengue-2/3 chimera or DENV-2/3 chimera, or TDV-3 has one further mutation at said corresponding prM-E gene from wild-type DEN-3 16562 such that the amino acid position 223 of the E protein contains a serine residue, and wherein the dengue-2/4 chimera or DENV-2/4 chimera, or TDV-4 has two further mutations such that the amino acid position 66 of the NS2A protein contains a glycine residue and the amino acid position 21 of the NS4A protein contains a valine residue and wherein the dengue-2/4 chimera or DENV-2/4 chimera, or TDV-4 is a mixed genotype with respect to amino acid position 99 of the NS2A protein containing an arginine or a lysine residue.

Thus, the dengue-2/1 chimera or DENV-2/1 chimera, or TDV-1 has said DEN-2 PDK-53 genome as viral backbone and has the prM-E gene (nt-457 to -2379) of said DEN-2 PDK-53 genome replaced with the corresponding prM-E gene from wild-type DEN-1 16007, wherein the dengue-2/3 chimera or DENV-2/3 chimera, or TDV-3 has said DEN-2 PDK-53 genome as viral backbone and has the prM-E gene (nt-457 to -2373) of said DEN-2 PDK-53 genome replaced with the corresponding prM-E gene from wild-type DEN-3 16562, and wherein the dengue-2/4 chimera or DENV-2/4 chimera, or TDV-4 has said DEN-2 PDK-53 genome as viral backbone and has the prM-E gene (nt-457 to -2379) of said DEN-2 PDK-53 replaced with the corresponding prM-E gene from wild-type DEN-4 1036.

Thus, the live, attenuated dengue-2 virus serotype or DENV-2 or TDV-2 is represented by a polynucleotide of SEQ ID NO: 3, or a polypeptide of SEQ ID NO: 4; the dengue 2/1 chimera or DENV-2/1 chimera, or TDV-1 having nonstructural proteins from a modified live, attenuated dengue-2 virus serotype and structural proteins from a dengue-1 virus serotype, represented by a polynucleotide of SEQ ID NO: 1, or a polypeptide of SEQ ID NO: 2; the dengue 2/3 chimera or DENV-2/3 chimera, or TDV-3 having nonstructural proteins from a modified live, attenuated dengue-2 virus serotype and structural proteins from a dengue-3 virus serotype, represented by a polynucleotide of SEQ ID NO: 5, or a polypeptide of SEQ ID NO: 6; and the dengue 2/4 chimera or DENV-2/4 chimera, or TDV-4 having nonstructural proteins from a modified live, attenuated dengue-2 virus serotype and structural proteins from a dengue-4 virus serotype, represented by a polynucleotide of SEQ ID NO: 7, or a polypeptide of SEQ ID NO: 8.

An RNA virus, as comprised in TDV, can be characterized by its RNA sequence. It is, alternatively or in addition, also common practice to characterize the genome of RNA viruses by the corresponding DNA sequences and corresponding DNA sequences are readily understood to reflect the RNA genome in the virus. Therefore, reference to “t” or “thymine” in the entire disclosure is to be understood as reference to “u” or “uracil”, respectively, if the genomic RNA virus sequence is meant

### Providing Cells in Growth Media

According to the invention, the method for large-scale flaviviral vaccine production and manufacture preferably comprises a step of providing cells in growth media. In a preferred embodiment, the flaviviral vaccine is a tetravalent dengue vaccine including all four live, attenuated dengue serotypes including dengue serotype 1 such as dengue 2/1 chimera or TDV-1 represented by SEQ ID NO: 1 and/or SEQ ID NO:2, dengue serotype 2 or TDV-2 represented by SEQ ID NO: 3 and/or SEQ ID NO:4, dengue serotype 3 such as dengue serotype 2/3 or TDV-3 represented by SEQ ID NO: 5 and/or SEQ ID NO:6, dengue serotype 4 such as dengue serotype 2/4 or TDV-4 represented by SEQ ID NO: 7 and/or SEQ ID NO: 8. The methods described in this section are performed for each of the four serotypes separately.

This step further involves expansion of cells to sufficient numbers in growth media. For the purpose of large-scale flaviviral vaccine production and manufacture, a number of different cell lines can be used including Madin-Darby Canine Kidney cells, monkey cell lines pMK, Vero, and human cell lines HEK 293, MRC 5, Per.C6, PMK, LLCMK2, BHK and WI-38. All of these cell lines are well characterized with a proven safety profile. Furthermore, these cell lines are suitable to support replication of the attenuated vaccine strains. Each of these cell lines represents a separate embodiment of the invention.

Accordingly, for the purpose of the present invention a cell line selected from Madin-Darby Canine Kidney cells, monkey cell lines pMK, Vero, and human cell lines HEK 293, MRC 5, Per.C6, PMK, LLCMK2, BHK, and WI-38 can be used to develop two cell banks, a master cell bank (MCB) and a working cell bank (WCB). As used herein the term “master cell bank” refers to a culture of cells (e.g., fully characterized cells) that have been grown from a single clone and stored under cryopreservation conditions. The cells from the MCB are used to develop a working cell bank. As used herein the term “working cell bank”, refers to a culture of cells (e.g., fully characterized cells) that has been grown from a single vial of the MCB, or from two pooled vials of the master cell bank and stored under cryopreservation conditions. The cells from the WCB are later used in a production cell culture.

The method of the present invention also includes the use of growth media and additive formulations for the expansion of the cells, which may or may not be the same as the media in which the WCB and MCB are developed. According to the present invention, the growth media used for the expansion of cells comprises one or more of growth media, glutamine or GlutaMAX™, and serum.

The term “growth media” refers to a medium for culturing cells containing nutrients that maintain cell viability and support proliferation. The growth media may contain any of the following nutrients in appropriate amounts and combination: salt(s), buffer(s), amino acids, glucose or other sugar(s), antibiotics, and other components such as peptide growth factors, etc. “Growth media” are known in the art and may be classified as natural or artificial cell culture media. Examples of cell culture media that can be used for the method of the present invention include Minimum Essential Medium (MEM), Dulbecco's Modified Eagle's Medium (DMEM), DMEM/F-12, and Roswell Park Memorial Institute Medium (RPMI). Different culture media having different ranges of pH, glucose concentration, growth factors, and other supplements that can be used for different cell types or for different applications. In some embodiments, custom cell culture media or commercially available cell culture media such as Dulbecco's Modified Eagle Medium, Minimum Essential Medium, RPMI medium, HA or HAT medium, or other media available from other commercial sources can be used. The growth media can include one or more antibiotics (e.g., actinomycin D, ampicillin, carbenicillin, cefotaxime, fosmidomycin, gentamycin, kanamycin, neomycin, penicillin, penicillin streptomycin, polymyxin B, streptomycin, tetracycline, or any other suitable antibiotic or any combination of two or more thereof). The growth media can include one or more salts (e.g., balanced salts, calcium chloride, sodium chloride, potassium chloride, magnesium chloride, etc.). The growth media can also include one or more buffers (e.g., HEPES or other suitable buffer). The growth media can also include differentiation factors. Growth or differentiation factors (e.g., WNT-family proteins, BMP-family proteins, IGF-family proteins, etc.) can be added individually or in combination, e.g., as a differentiation cocktail including different factors that bring about differentiation toward a particular lineage. It is understood that each individual possibility can be combined with another possibility to arrive at a combination of features and that each possibility, and each combination represents a separate embodiment of the invention.

The concentration of each of the individual components in the growth media can be adapted to the needs of the cell line used and the flaviviral vaccine to be produced. For example, the concentration of glucose can be between 3 g/L and 4 g/L of glucose, 3.1 to 3.2 g/L, 3.2 to 3.3 g/L, 3.3 to 3.4 g/L, 3.4 to 3.5 g/L, 3.5 to 3.6 g/L, 3.6 to 3.7 g/L, 3.7 to 3.8 g/L, 3.8 to 3.9 g/L, or 3.9 to 4.0 g/L. The concentration of glucose can be, for example, 3.12 g/L, 3.22 g/L, 3.32 g/L, 3.42 g/L, 3.52 g/L, 3.62 g/L, 3.72 g/L, 3.82 g/L, or 3.92 g/L. Each of these possibilities represents a separate embodiment of the invention.

The concentration of glutamine or GlutaMAX™, if added, can be between 3.5 mM to 4.5 mM of glutamine or GlutaMAX™, 3.5 mM to 3.6 mM, 3.6 mM to 3.7 mM, 3.7 mM to 3.8 mM. 3.8 mM to 3.9 mM, 4.0 mM to 4.1 mM, 4.1 to 4.2 mM, 4.2 to 4.3 mM, 4.3 to 4.4 mM, or 4.4 to 4.5 mM. The concentration of glutamine or GlutaMAX™ can be, for example 3.5 mM, 3.6 mM, 3.7 mM, 3.8 mM, 3.9 mM, 4.0 mM, 4.1 mM, 4.2 mM, 4.3 mM, 4.4 mM, or 4.5 mM. Each of these possibilities represents a separate embodiment of the invention.

“Serum” as used herein refers to mammalian serum and can be selected from human serum, equine serum, bovine serum, or sheep serum. Serum is widely used as a supplement of the cell growth media for adherent mammalian cell culture as it provides a wide variety of macromolecular proteins, low molecular weight nutrients, carrier proteins for water-insoluble components, and other compounds necessary for in vitro growth of cells, such as hormones and attachment factors. The term “serum” further encompasses a solution based on serum containing a buffer. The addition of substances like salts, buffers, sugars, a chelating agents, preservatives and protease inhibitors to the serum used in the present invention can be included. The term “equine serum” as used herein refers to serum obtained from horses. The term “bovine serum” as used herein refers to serum obtained from bovine, commonly used bovine serum is termed “fetal bovine serum (FBS)” in the art. The term “sheep serum” as used herein refers to serum obtained from sheep. The term “human serum” as used herein refers to serum obtained from human. In some embodiments, cell culture media include serum (e.g., fetal bovine serum, bovine calf serum, equine serum, porcine serum, or other serum). In some embodiments, cell culture media are serum-free. In some embodiments, cell culture media include human platelet lysate (hPL).

If serum is added, the concentration of serum can be in the range 1 to 10%, for example between 1 to 2%, 2 to 3%, 3 to 4%, 4 to 5%, 5 to 6%, 6 to 7%, 7 to 8%, 8 to 9%, or 9 to 10%. The concentration of serum can be, for example, 1%, 2%, 3%, 4%, 5%, 6%, 7%, 8%, 9%, or 10%. Thus, the concentration of FBS in the media can be in the range 1 to 10%, for example between 1 to 2%, 2 to 3%, 3 to 4%, 4 to 5%, 5 to 6%, 6 to 7%, 7 to 8%, 8 to 9%, or 9 to 10%. Thus, the concentration of equine serum in the media can be in the range 1 to 10%, for example between 1 to 2%, 2 to 3%, 3 to 4%, 4 to 5%, 5 to 6%, 6 to 7%, 7 to 8%, 8 to 9%, or 9 to 10%. Thus, the concentration of sheep serum in the media can be in the range 1 to 10%, for example between 1 to 2%, 2 to 3%, 3 to 4%, 4 to 5%, 5 to 6%, 6 to 7%, 7 to 8%, 8 to 9%, or 9 to 10%. Thus, the concentration of porcine serum in the media can be in the range 1 to 10%, for example between 1 to 2%, 2 to 3%, 3 to 4%, 4 to 5%, 5 to 6%, 6 to 7%, 7 to 8%, 8 to 9%, or 9 to 10%. Each of these possibilities represents a separate embodiment of the invention.

Further optional constituents of the growth media for the purpose of the invention include a pH indicator. The pH indicator that can be used for the method of the invention can be phenol red. In the presence of phenol red, microbial contamination of the media might be indicated by a shift to lower pH values, which will trigger a color change of the pH indicator. Instead of or in addition to the pH indicator, visual microscopic check for microbial contamination can be implemented. Each possibility has been tested and represents a separate embodiment of the invention.

The growth media may or may not include a non-ionic surfactant such as a poloxamer. Preferably, the growth media does not comprise a poloxamer. Thus, according to one embodiment of the invention, the method for large-scale flaviviral vaccine production and manufacture comprises a step of providing cells in growth media, wherein the growth media does not comprise a non-ionic surfactant such as a poloxamer.

In the context of the invention, growth media preparation strategy is also a factor to control microbial growth. For the purpose of the invention, the growth media can be prepared by either “aseptically mixing” and homogenizing all components, or by mixing the components and then “filtering” them depending on the virus that is being produced. The term “aseptic” is understood to mean a condition in which there is substantially no growth of unwanted or pathogenic organisms and substantially no build-up of debris or other medium in which such organisms are likely to reproduce.

The invention includes preparing the growth media in any of the two ways described above. Further, the invention includes using one way of preparing the growth media for one flavivirus and another way for another flavivirus. Further, if a flavivirus with different serotypes is being produced, the invention includes producing one serotype in growth media prepared in one way and another serotype in growth media prepared in another way. For example, for the purpose of the invention, if dengue serotypes are being separately produced, serotype 4 may be produced with a filtered growth media while serotype 1 may be produced with aseptic mixing and vice versa. Each possibility represents a separate embodiment of the invention. When filtration is used, preferably a filter less than 1 μm is used, more preferably a filter less than 0.5 μm is used.

Another optional additive that can be included is a dissociation reagent for adherent cell passaging to dislodge the cells from the surface. The term “dissociation reagent” as used herein refers to a solution or fluid which is contacted with anchorage-dependent cells and causes the cells to dissociate (loosen their cell attachment and may become detached) from the surface to which they adhere. Thus, if for the purpose of the invention adherent cells are used, the growth media can also comprise dissociation reagents such as one or more chelators (ethylenediamine tetraacetate, “EDTA”; ethylene glycol-bis beta-aminoethyl ether N,N,N′,N′-tetraacetic acid, “EGTA”; versen; and the like). The growth media can alternatively comprise one or more proteolytic enzymes (e.g., ficin, pepsin, trypsin, chymotrypsin, papain, and the like, with trypsin being a preferred enzyme). The proteolytic enzymes used in the present invention can be recombinant e.g., commercially available TrypLE Select or of mammalian origin e.g., trypsin of porcine origin. The growth media can also comprise a combination of chelators and proteolytic enzymes. Each possibility represents a separate embodiment of the invention.

The targeted volume of the dissociation reagent is determined by the surface area of the culture vessel. The volume to surface area ratio of the dissociation reagent can be between 0.01 to 0.02 mL/cm², or 0.011 to 0.016 mL/cm². Thus, for example, the volume to surface area ratio of proteolytic enzymes can be between 0.01 to 0.02 mL/cm², or 0.011 to 0.016 mL/cm². Thus, for example, the volume to surface area ratio of pepsin can be between 0.01 to 0.02 mL/cm², or 0.011 to 0.016 mL/cm². Thus, for example, the volume to surface area ratio of TrypLE Select can be between 0.01 to 0.02 mL/cm², or 0.011 to 0.016 mL/cm². Thus, for example, the volume to surface area ratio of trypsin of porcine origin can be between 0.01 to 0.02 mL/cm², or 0.011 to 0.016 mL/cm².

The duration of treatment with the dissociation agent can be up to 100 minutes, up to 90 minutes, up to 80 minutes, up to 70 minutes, up to 60 minutes, up to 50 minutes, up to 40 minutes, up to 30 minutes, up to 20 minutes. Thus, for example, the duration of treatment with a proteolytic enzyme can be up to 100 minutes, up to 90 minutes, up to 80 minutes, up to 70 minutes, up to 60 minutes, up to 50 minutes, up to 40 minutes, up to 30 minutes, up to 20 minutes. Thus, for example, the duration of treatment with TrypLE select can be up to 100 minutes, up to 90 minutes, up to 80 minutes, up to 70 minutes, up to 60 minutes, up to 50 minutes, up to 40 minutes, up to 30 minutes, up to 20 minutes. Thus, for example, the duration of treatment with pepsin can be up to 100 minutes, up to 90 minutes, up to 80 minutes, up to 70 minutes, up to 60 minutes, up to 50 minutes, up to 40 minutes, up to 30 minutes, up to 20 minutes. Thus, for example, the duration of treatment with trypsin of porcine origin can be up to 100 minutes, up to 90 minutes, up to 80 minutes, up to 70 minutes, up to 60 minutes, up to 50 minutes, up to 40 minutes, up to 30 minutes, up to 20 minutes.

If a dissociation reagent is used and the growth media comprises serum, preferably the cells are washed prior to using the dissociation reagent. Preferably, the wash is conducted once, twice, or thrice before adding the dissociation reagent. The wash can be conducted using any of the wash buffers such as phosphate-buffered saline (PBS), Dulbeco's phosphate-buffered saline (DPBS) or Tris buffered saline (TBS). “PBS” as used herein is a water-based salt solution containing disodium hydrogen phosphate, sodium chloride and, in some formulations, potassium chloride and potassium dihydrogen phosphate. “Dulbecco's phosphate-buffered saline (DPBS)” as used herein is a balanced salt solution used for a variety of cell culture applications, such as washing cells before dissociation, transporting cells or tissue samples, diluting cells for counting, and preparing reagents.

When all the cells are dislodged from the surface of the culture vessel, the cells are washed with the growth media that was used for cell expansion. The targeted volume of the growth media used per wash is determined by the surface area of the culture vessel. The volume to surface area ratio of the wash with the growth media can be between 0.04 to 0.06 mL/cm² or 0.046 to 0.053 mL/cm².

In the context of the invention, it is to be understood that each of the individual components of the media described herein above are contemplated alone or in combination with each other based on the cell line used and the virus to be produced. These components provide all the necessary nutrients, growth factors and other proteins for adherent cell growth on the surface of the culture vessel.

For the purpose of the present invention, operating ranges that support robust cell growth for large-scale production and manufacture of flaviviral vaccines have to be used.

One of the parameters is the cell thawing process. The term “thawing” refers to raising the temperature of the cryopreserved composition or biological material in this case cells, to 0° C. or above, preferably to 4° C. or above. The term “thawing” may also refer to raising the temperature of the cryopreserved composition or biological material to a temperature at which there are no or substantially no ice crystals in all or part of the cryopreserved composition or biological material. Hence the term “thawing” includes complete and partial thawing. The duration of the cell thawing process can be within the range of 1 to 5 minutes, or 3 to 5 minutes. Thus, for example, when the cells used are Vero cells, the duration of thawing can be within the range of 1 to 5 minutes, or 3 to 5 minutes. After the thawing process, the post-thawed cells can be stored in ambient temperature. “Ambient temperature” as used herein refers to room temperature that is between 20 to 22° C. The duration of storage of the post-thawed cells can be extended up to 5, 6, 7, or 8 minutes. Thus, for example, when the cells used are Vero cells, the duration of storage of the post-thawed cells can be extended up to 5, 6, 7, or 8 minutes.

Other parameters are the percentage of carbon dioxide (CO₂) and temperature during the incubation of cells in the expansion process. For the purpose of the invention, CO₂ percentage can be between 2 to 8%, 4% to 6%, or less than 6%, such as, for example, 5% or 6%, and a temperature range between 36° C. to 39° C. temperature, or less than 39° C., such as for example, 37° C. or 38° C.

Another parameter is to determine the allowable time for the cells to be left “dry” (that is when adherent cells are used, cells monolayer to be without coverage of solution, such as after growth media or DPBS removal) during passaging. Said time can be up to two hours, preferably less than two hours such as, for example, 30 minutes, 45 minutes, 60 minutes. As used herein, the term “passaging” refers to transferring a cell or cells from a first growth environment to a second growth environment, wherein the cell density of the second is less than that of the first.

Another parameter is the maximum process duration for passaging, i.e., the time period starting from when the cells are out of the incubator, passaged and are on hold with growth media. The maximum process duration for passaging can be up to 20 hours, preferably less than 20 hours, or less than 15 hours or less than 10 hours such as, for example, 5 hours, 6 hours, 7 hours, 8 hours or 9 hours.

Another parameter is the maximum cell split ratio, which can be set up to 1:8 irrespective of the culture vessel used. This parameter largely depends on the cell density and confluency up to which the cells are expanded before infection. For the method of the present invention, the minimum confluency of cells before infection is between 80 to 90%, preferably more than 80%. Accordingly, for the method of the present invention, the cell seeding density is in the range 1.5×10⁴ to 3×10⁴ cells/cm², such as, for example, 1.75×10⁴ or 2×10⁴ cells/cm² and the cell density before infection is the range of 9×10⁴ to 2×10⁵ cells/cm².

Thus, for large-scale flaviviral vaccine production and manufacture, the duration of the cell thawing process can be within the range of 3 to 10 minutes, the incubation CO₂ percentage can be between 2 to 8%, the dry period of the cells can be two hours, the maximum process duration for passaging can be up to 20 hours, and the maximum cell split ratio can be up to 1:8. Thus, for large-scale flaviviral vaccine production and manufacture the duration of the cell thawing process can be within the range of 3 to 8 minutes, the incubation CO₂ percentage can be between 4% to 6%, the dry period of the cells can be less than two hours, the maximum process duration for passaging can be up to 20 hours, and the maximum cell split ratio can be up to 1:8. Thus, for large-scale flaviviral vaccine production and manufacture the duration of the cell thawing process can be within the range of 3 to 5 minutes, the incubation CO₂ percentage can be less than 6%, the dry period of the cells can be less than two hours, the maximum process duration for passaging can be up to 20 hours, and the maximum cell split ratio can be up to 1:8.

Accordingly, in one embodiment of the method for large-scale production and manufacture of flaviviral vaccines, includes a step of providing cells in growth media without a poloxamer comprising expansion of cells to sufficient numbers. In the context of the invention, it is to be understood that each of the individual method steps and/or components described herein above are contemplated alone or in combination with each other. Thus, each possibility described herein above alone and in combination with each other represents a separate embodiment of the invention.

### Infection of Cells

According to one embodiment of the invention, the method for large-scale flaviviral vaccine production and manufacture comprises the sequential steps of (i) providing cells in growth media, (ii) infecting the cells of step (i) with media comprising a flavivirus at a low MOI. In this context, the cells can be Vero cells. In this context, the flavivirus can be a dengue virus. In this context, the vaccine can be a live, attenuated viral vaccine. In a preferred embodiment, the flaviviral vaccine is a tetravalent dengue vaccine including all four live, attenuated dengue serotypes including dengue serotype 1 such as dengue 2/1 chimera or TDV-1 represented by SEQ ID NO: 1 and/or SEQ ID NO:2, dengue serotype 2 or TDV-2 represented by SEQ ID NO: 3 and/or SEQ ID NO:4, dengue serotype 3 such as dengue serotype 2/3 or TDV-3 represented by SEQ ID NO: 5 and/or SEQ ID NO:6, dengue serotype 4 such as dengue serotype 2/4 or TDV-4 represented by SEQ ID NO: 7 and/or SEQ ID NO:8. The methods described in this section are performed for each of the four serotypes separately.

After the cells have been expanded to sufficient numbers in production vessels as explained in the previous section, the cells are infected with media comprising the working virus seed (WVS) comprising flavivirus. This media is called the infection media. The infection media may or may not be the same as the growth media. The objective of this step is to ensure that the virus infects the cells and replicates to a sufficient potency for harvesting and purification. Furthermore, when an attenuated virus is used, the additional objective is that the virus retains its attenuating loci. The WCB is used for the manufacture of the master and working virus seeds, MVS and WVS, respectively. As used herein the term “master virus seeds (MVS)” within the meaning of this disclosure is a virus that is intended to be used for the production of a vaccine. MVS is first used to inoculate the WVS, which is then used to inoculate the production culture.

As used herein, “production vessels” refers to “culture vessels” that are inoculated with infection media comprising the WVS for the purpose of large-scale production and manufacture of flaviviral vaccines. The term “culture vessel” may refer to any container in which cells may be cultured. Culture vessels include, but are not limited to, tissue culture flasks, 96 well plates, culture dishes, culture slides, and rotating wall vessels. For the purpose of the invention, tissue culture flasks that can be used are Corning® CellSTACK® culture chambers or Nunc™ EasyFill™ Cell Factory™ systems. For example, the Corning® CellSTACK® culture chambers that can be used alone or in combination are 1-stack (CF1) with 636 cm² cell growth area, 2-stack (CF2) with 1,272 cm² cell growth area, 5-stack (CF5) with 3,180 cm² cell growth area, 10-stack (CF10) with 6,360 cm² cell growth area, 40-stack (CF40) with 25,440 cm² cell growth area.

For the purpose of the invention, maintaining cell viability while facilitating the viral replication in cells as well as the stability of the produced virus are particularly ensured. Thus, all the method steps of the invention are carried out to specifically attain these advantages. The method of the present invention also includes the use of infection media comprising a WVS comprising a flavivirus for infection of cells. Thus, the present invention includes the use of infection media comprising a flavivirus for infection of cells. In the context of the method of the invention, the infection media can be the same as the growth media used for the expansion of cells or it can be different.

The infection media can comprise one or more of growth media, and additionally, glutamine or GlutaMAX™, and a non-ionic surfactant.

The term “non-ionic surfactant” means a surfactant that contains neither positively nor negatively charged functional groups. In contrast to anionic and cationic surfactants, non-ionic surfactants do not ionize in solution. Non-ionic surfactants may be selected from block copolymers, sorbitan esters, ethoxylated or propoxylated sorbitan esters, alkyl-polyglycosides (APG), alkoxylated mono- or di-alkylamines, fatty acid monoethanolamides (FAMA), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), and combinations thereof.

The non-ionic surfactant can be a high molecular weight non-ionic surfactant. “High molecular weight” means a molecular weight of 1500 or more. The non-ionic surfactant can be a non-ionic triblock copolymer. The surfactant can be a non-ionic, hydrophilic, polyoxyethylene-polyoxypropylene block copolymer (or EO-PO block copolymer). The EO-PO block copolymers can include blocks of polyethylene oxide (—CH2CH2O-designated EO) and polypropylene oxide (—CH2CHCH3O-designated PO). The PO block can be flanked by two EO blocks in a EOx-POy-Eox arrangement. Since the PO component is hydrophilic and the EO component is hydrophobic, the overall hydrophilicity, molecular weight and the surfactant properties of the copolymer can be adjusted by varying x and y in the EOx-POy-Eox block structure. In aqueous solutions, the EO-PO block copolymers will self-assemble into micelles with a PO core and a corona of hydrophilic EO groups.

The non-ionic surfactant can be a poloxamer. Poloxamers are non-ionic triblock copolymers composed of a central hydrophobic chain of poly(propyleneoxide) flanked by two hydrophilic chains of poly(ethylene oxide). The length of the polymer blocks can be customized, leading to different poloxamers with slightly different properties.

As used herein, “EO-PO block copolymer” can mean a copolymer consisting of blocks of poly(ethylene oxide) and poly(propylene) oxide. In addition, as used herein, “Pluronic” can mean EO-PO block copolymers in the EOx-POy-EOx. This configuration of EO-PO block copolymer is also referred to as “Poloxamer”.

Accordingly, the non-ionic surfactant can be Pluronic F127 (poloxamer 407), Pluronic F123 (poloxamer 403), Pluronic F-68 (poloxamer 188), Pluronic P123, Pluronic P85, other polyethylene oxide-polypropylene oxide (EO-PO) block copolymers of greater than 3,000-4,000 MW or combinations thereof. “Poloxamer 407” as used herein is a hydrophilic non-ionic surfactant which consists of a triblock copolymer consisting of a central propylene glycol block with about 56 repeat units and two flanking hydrophilic polyethylene glycol blocks each comprising about 101 repeat units. “Poloxamer 407” is also known by its trade names Pluronic F127 and Synperonic PE/F127. “Poloxamer 188” as used herein (P188) is a non-ionic linear copolymer having an average molecular weight of 8400 Daltons and is also referred to as Pluronic F68, FLOCOR and RheothRx. Pluronic P123 is a symmetric triblock copolymer comprising poly(ethylene oxide) (PEO) and poly(propylene oxide) (PPO) in an alternating linear fashion, PEO-PPO-PEO. Pluronic P85 is a difunctional block copolymer surfactant terminating in primary hydroxyl groups.

Thus, according to one embodiment of the invention, the preparation of the infection media can include adding glutamine or GlutaMAX™ and non-ionic surfactant, resulting in specific concentrations of glucose, glutamine or GlutaMAX™ and non-ionic surfactant in the infection media. The concentration of glucose in the infection media can be between 3 g/L and 4 g/L of glucose, 3.1 to 3.2 g/L, 3.2 to 3.3 g/L, 3.3 to 3.4 g/L, 3.4 to 3.5 g/L, 3.5 to 3.6 g/L, 3.6 to 3.7 g/L, 3.7 to 3.8 g/L, 3.8 to 3.9 g/L, or 3.9 to 4.0 g/L. The concentration of glucose can be, for example, 3.11 g/L, 3.22 g/L, 3.33 g/L, 3.44 g/L, 3.55 g/L, 3.66 g/L, 3.77 g/L, 3.88 g/L, or 3.99 g/L. Each of these possibilities represents a separate embodiment of the invention.

The concentration of glutamine or GlutaMAX™ in the infection media can be between 3.5 mM to 4.5 mM, 3.5 mM to 3.6 mM, 3.6 mM to 3.7 mM, 3.7 mM to 3.8 mM. 3.8 mM to 3.9 mM, 4.0 mM to 4.1 mM, 4.1 to 4.2 mM, 4.2 to 4.3 mM, 4.3 to 4.4 mM, or 4.4 to 4.5 mM. The concentration of glutamine or GlutaMAX™ can be, for example 3.5 mM, 3.6 mM, 3.7 mM, 3.8 mM, 3.9 mM, 4.0 mM, 4.1 mM, 4.2 mM, 4.3 mM, 4.4 mM, or 4.5 mM. Each of these possibilities represents a separate embodiment of the invention.

For carrying out certain embodiments of the method of the invention, the addition of non-ionic surfactant at specific concentrations enhances the viral production compared to infection media without the addition of non-ionic surfactant. For example, the concentration of the non-ionic surfactant can be between 0.05% to 2.0% (w/v) in the medium. For example, 0.05%, 0.1%, 0.15%, 0.20%, 0.25%. 0.3%, 0.35%, 0.4%, 0.45% or 0.5% (w/v). Each possibility represents a separate embodiment of the invention. For example, the concentration of poloxamer 407 and/or F127 can be between 0.05% to 2.0% (w/v) in the medium. For example, 0.05%, 0.1%, 0.15%, 0.20%, 0.25%. 0.3%, 0.35%, 0.4%, 0.45% or 0.5% (w/v). Each possibility represents a separate embodiment of the invention. For example, the concentration of poloxamer 403 and/or F68 can be between 0.05% to 2.0% (w/v) in the medium. For example, 0.05%, 0.1%, 0.15%, 0.20%, 0.25%. 0.3%, 0.35%, 0.4%, 0.45% or 0.5% (w/v). Each possibility represents a separate embodiment of the invention. For example, the concentration of P123 can be between 0.05% to 2.0% (w/v) in the medium. For example, 0.05%, 0.1%, 0.15%, 0.20%, 0.25%. 0.3%, 0.35%, 0.4%, 0.45% or 0.5% (w/v). Each possibility represents a separate embodiment of the invention. For example, the concentration of P85 can be between 0.05% to 2.0% (w/v) in the medium. For example, 0.05%, 0.1%, 0.15%, 0.20%, 0.25%. 0.3%, 0.35%, 0.4%, 0.45% or 0.5% (w/v). Each possibility represents a separate embodiment of the invention.

The infection media with and without a pH indicator can be used. The pH indicator for the infection media can be phenol red. If no pH indicator is used, e.g., the media does not contain phenol red, visual microbial assessment may be implemented.

In the context of the invention, infection media preparation strategy is also a factor to control microbial growth. The way the infection media is prepared can affect the growth or potency of the virus. For the purpose of the invention, the infection media can be prepared by either “aseptically mixing” and homogenizing all components or mixing the components and then “filtering” them depending on the flavivirus that is being produced. The invention includes preparing the infection media in any of the two ways described above.

Further, the invention includes using one way of preparing the infection media for one flavivirus and another way for another flavivirus. Further, if a virus with different serotypes is being used, the invention includes producing one serotype in infection media prepared in one way and another serotype in infection media prepared in another way. For example, for the purpose of the invention, if dengue serotypes are being separately produced, serotype 4 may be produced with a filtered infection media while serotype 1 may be produced with aseptic mixing and vice versa. Each possibility represents a separate embodiment of the invention. When filtration is used, preferably a filter less than 1 μm is used, more preferably a filter less than 0.5 μm is used.

The cells provided in growth media are infected with media comprising a WVS of the flavivirus. This means that each flavivirus and each serotype of each flavivirus has its own WVS from which a separate production culture for large-scale production and manufacture of the flaviviral vaccine is initiated. Infection is a complex biological process which involves various operating parameters that will impact the infection efficiency/harvest potency, namely viruses attaching, entering and infecting the cells.

It has been surprisingly found that the infection parameters used for the method of the invention are reproducible and allow large-scale production of flaviviruses in general as well as large-scale production of attenuated flaviviruses.

For the purpose of achieving high infection efficiency/high harvest potency/and/or high viral titers, the method of the invention includes specific infection parameters.

One of the infection parameters is the multiplicity of infection (“MOI”). MOI refers to the ratio defined by the number of infectious virus particles deposited in a vessel and/or container divided by the number of target cells present in that vessel and/or container.

The probability that a given infectious virus particle infects a target cell is given by a Poisson's distribution,

$$P ( n ) = \frac{{m}^{n} \cdot {e}^{- m}}{n !}$$

i.e., the infections are independent of each other.
m is the multiplicity of infection or MOI,
n is the number of infectious virus particles, and
P(n) is the probability that a target cell will get infected by n infectious virus particles.

If n=0, the probability that a target cell will not get infected can be calculated. Thus, the probability that a target cell will get infected by a virus particle is 1−P(0).

$$P ( n > 0 ) = 1 - P ( n = 0 ) = 1 - \frac{{m}^{0} \cdot {e}^{- m}}{0 !} = 1 - {e}^{- m}$$

Thus, the average percentage of cells that will become infected as a result of inoculation with a given MOI is given by the above equation. For example, for MOI=1, 63.2% of cells will get infected. Thus, a skilled person understands that the MOI would also impact the efficiency of virus production that is calculated in terms of viral titers.

The terms “infection efficiency” or “transduction efficiency” or “virus potency” or “viral titers” are used interchangeably and refer to the ability of a flaviviral particle to bind to, penetrate and deliver its genome to the cytoplasm of a host cell, thereby allowing expression of structural proteins in a host cell. The infection efficiency can be measured using analytical techniques known in the art such as an immunofocus assay (“IFA assay”). The IFA assay is a well-known analytical technique used in the art. The principle of the assay is based on classical virus plaque assays where serial dilutions of virus are plated on monolayers of adherent cells from a suitable host. After a period of time to allow infectious virions to bind and be taken up by cells, an overlay medium containing thickening agents is added to prevent diffusion of virions. Therefore, progeny virions can only infect cells adjacent to the original infected cell. This results in a roughly circular focus of infection for each infectious unit of virus. Immunofocus assays differ from the classical plaque assay in that foci of infection are detected by immunostaining instead of visual observation of cytopathic effect. In particular, the term “viral titre” refers to the number of infectious viral particles, or “transducing units,” that result in the infection of a target cell. Viral titer in the context of the invention can be measured using the IFA assay discussed herein above. Viral titers determined by an IFA assay result in plaque forming units (PFU). In one example, the immune focus assay can be carried out as described in detail in section 2.5 of Brewoo et al. (Vaccine. 2012 Feb. 14; 30(8): 1513-1520. doi:10.1016/j.vaccine.2011.11.072) Alternatively, any other analytical method can be used to measure the viral titer, for example, viral titer can be measured by a functional assay, such as an assay described in Xiao et al., Exp. Neurobiol. 144:113-124, 1997, or Fisher et al., J. Virol. 70:520-532, 1996, the disclosures of both of which are incorporated herein by reference.

“High viral titers” as used herein refers to a viral titre greater than 7.0 log 10 PFU/mL, preferably greater than 7.5 log 10 PFU/mL.

For large-scale vaccine production and manufacture, one of the biggest challenges is to conserve the MVS and WVS stocks from an economic point of view and at the same time obtaining high viral titres for meeting the requirements of the population in a timely manner. This challenge is further exacerbated in large-scale production and manufacture of attenuated viral vaccines because an attenuated virus may have a compromised replication rate.

It has been surprisingly found that for the method of the invention, an infection media comprising a flavivirus at a low MOI can be used for large-scale flaviviral vaccine production and manufacture. Even more surprisingly, it has been found that a low MOI can be used for large-scale attenuated flaviviral vaccine production and manufacture. In this context, the flavivirus can be a dengue virus. In one embodiment, by using the method of the invention, a low MOI can be used for large-scale flaviviral vaccine production and manufacture providing higher average viral titres as compared to the same method comprising the same process steps but using a high MOI. Thus, by using the method of the invention, a low MOI can be used for large-scale attenuated flaviviral vaccine production and manufacture providing higher average viral titers as compared to the same method comprising the same process steps but using a high MOI. In this context, high MOI refers to MOI greater than 0.008. In this context, the flavivirus can be a dengue virus. “Average viral titers” as used herein refers to average viral titers of at least two harvesting steps.

“Low MOI” as used herein refers to MOI of 0.008 and less such as less than 0.008, or 0.005 or less such as less than 0.005, preferably, from 0.0001 to 0.008, or from 0.0001 to 0.005 such as, from 0.001 to 0.005, e.g., 0.0011, 0.0012, 0.0013, 0.0014, 0.0015, 0.002, 0.0021, 0.0022, 0.0023, 0.0024, 0.0025, 0.003, 0.0031, 0.0032, 0.0033, 0.0034, 0.0035, 0.004, 0.0041, 0.0042, 0.0043, 0.0044, 0.0045, or 0.005. Accordingly, for the purpose of the present invention, a low MOI within the range of 0.008 or less can be used. Accordingly, for the purpose of the present invention, a low MOI within the range of 0.005 or less can be used. For the method of the invention, if a flavivirus with different serotypes is being used, the infection of each of the serotypes can be carried out within the low MOI range. It is also possible that one serotype is infected with media comprising a flavivirus at a certain low MOI within the range of 0.008 or less and 0.005 or less, and another serotype is infected with media comprising a flavivirus at another low MOI within the range of 0.008 or less and 0.005 and less. Each possibility represents a separate embodiment of the invention.

Thus, by using the method of the invention, a low MOI can be used for large-scale flaviviral vaccine production and manufacture providing high viral titres. Thus, by using the method of the invention, a low MOI can be used for large-scale attenuated flaviviral vaccine production and manufacture with high viral titers. In this context, the flavivirus can be a dengue virus. In one embodiment, by using the method of the invention, a low MOI can be used for large-scale flaviviral vaccine production and manufacture providing higher average viral titres as compared to the same method comprising the same process steps but using a high MOI. Thus, by using the method of the invention, a low MOI can be used for large-scale attenuated flaviviral vaccine production and manufacture providing higher average viral titers as compared to the same method comprising the same process steps but using a high MOI. In this context, high MOI refers to MOI greater than 0.008. In this context, the flavivirus can be a dengue virus. “Average viral titers” as used herein refers to average viral titers of at least two harvesting steps.

In one embodiment, vero cells in more than ten CF-10 flasks are infected with dengue serotype 1 such as dengue 2/1 chimera or TDV-1 represented by SEQ ID NO:1 and/or SEQ ID NO: 2 at a low MOI.

In one embodiment, vero cells in more than ten CF-10 flasks are infected with dengue serotype 2 or TDV-2 represented by SEQ ID NO:3 and/or SEQ ID NO: 4 at a low MOI.

In one embodiment, vero cells in more than ten CF-10 flasks or CF-40 flasks are infected with dengue serotype 3 such as dengue 3/1 chimera or TDV-3 represented by SEQ ID NO:5 and/or SEQ ID NO: 6 at a low MOI.

In one embodiment, vero cells in more than ten CF-40 flasks are infected with dengue serotype 4 such as dengue 2/4 chimera or TDV-4 represented by SEQ ID NO:7 and/or SEQ ID NO: 8 at a low MOI.

According to certain embodiments of the invention, the method for large-scale flaviviral vaccine production and manufacture comprises the sequential steps of (i) providing cells in growth media, and (ii) infecting the cells of step (i) with media comprising a flavivirus at a low MOI. Preferably, the growth media of step (i) does not comprise a non-ionic surfactant. In this context, the cells can be Vero cells. In this context, the flavivirus can be a dengue virus. In this context, the vaccine can be a live, attenuated viral vaccine.

In one embodiment, the large-scale flaviviral vaccine production and manufacture with high viral titers involves the use of a cell line that comprises abundant flaviviral receptors. In another embodiment, the large-scale attenuated flaviviral vaccine production and manufacture with high viral titers involves the use of a cell line that comprises abundant flaviviral receptors. In this context, the cells can be Vero cells. In this context, the flavivirus can be a dengue virus and the flaviviral receptors can be dengue receptors.

“Flaviviral receptors” as used herein refers to cell surface receptors. A wide range of cell surface receptors has been implicated in flavivirus entry into different cells types such as αvβ3 integrins C-type lectin receptors (CLR), phosphatidylserine receptors TIM (T-cell immunoglobulin and mucin domain) and TYRO3, AXL and MER (TAM). A cell line “abundant” in flaviviral receptors as used herein refers to a cell line that has some degree of overlap of flaviviral receptors with the flaviviral receptors in a mosquito cell line such as C636, for example, at least one or two common receptors.

“Dengue virus vaccine” as used herein means either a monovalent vaccine, i.e., one of the four dengue serotypes, divalent, vaccine, i.e., two of the four serotypes, trivalent vaccine, i.e., three of the four serotypes, or tetravalent vaccine, i.e., all four serotypes.

“Dengue virus receptors” as used herein refers to cell surface receptors. These include carbohydrate molecules, lectins, and claudin-1 cell receptors. Carbohydrate molecules such as glycosaminoglycans (GAGs), sulphated polysaccharides, and glycosphingolipids (GSL) are widely expressed cell surface co-receptors for DENV entry and are believed to enhance viral entry efficiency. A cell line “abundant” in dengue virus receptors as used herein refers to a cell line that has some degree of overlap of dengue virus receptors with the dengue virus receptors in a mosquito cell line such as C636, for example, at least one or two common receptors.

According to an aspect of the invention, the method for large-scale flaviviral vaccine production and manufacture comprises the sequential steps of (i) providing cells that comprise abundant flaviviral receptors in growth media, and (ii) infecting the cells of step (i) with media comprising a flavivirus at a low MOI. Preferably, the growth media of step (i) does not comprise a non-ionic surfactant. In this context, the cells can be Vero cells. In this context, the flavivirus can be a dengue virus. In this context, the vaccine can be a live, attenuated viral vaccine

Another infection parameter is the volume of infection media per surface area of the production vessel. For the method of the invention when low MOI range is used, the volume of infection media per surface area of the production vessel is such that it covers the monolayer of the cells within the tissue culture vessel. However, whether the infection media covers the cell monolayer or not can depend on the physical method of infection, i.e., either static or rocking.

Typically, for the purpose of large-scale vaccine production, it is understood that a rocking/shaking infection method should be used to ensure infection efficiency and virus potency. “Rocking/shaking as used herein refers to either manually rocking the tissue culture vessels or using a shaking platform. The term “static” infection means that the tissue culture vessel is not moving during the infection process and thus, the infection media is not mechanically dispersed throughout the cell monolayer. On the other hand, the term “rocking” infection means that the tissue culture vessel is kept on a platform that gently moves along a horizontal axis or the tissue culture vessel is manually moved so that the infection media is mechanically dispersed throughout the monolayer.

It has been surprisingly found that a static infection method can be used for large-scale flaviviral vaccine production and manufacture. Even more surprisingly, it has been found that a static infection method can be used for large-scale attenuated flaviviral vaccine production and manufacture. In this context, the flavivirus can be a dengue virus.

Thus, for large-scale flaviviral vaccine production and manufacture, static infection provides for the same infection efficiency and/or viral titers, i.e., high viral titers as compared to the rocking infection method. Thus, for large-scale flaviviral vaccine production and manufacture, static infection provides for the same infection efficiency and/or viral titers, i.e., high viral titers as compared to the rocking infection method when the cell monolayer is covered with infection media. The term “covered with infection media” refers to a state wherein the cell monolayer is completely or almost completely submerged in infection media. This would largely depend on the volume of infection media used and the tissue culture vessel used. Thus, whenever “static infection” is mentioned, it is understood that the volume of the infection media would be such that it covers the cell monolayer.

Furthermore, it has been surprisingly found that static infection in combination with low MOI range, provides for high infection efficiency and/or high viral titers for large-scale flaviviral vaccine production and even more surprisingly for large-scale attenuated flaviviral vaccine production. In this context, the flavivirus can be a dengue virus.

According to an aspect of the invention, the method for large-scale flaviviral vaccine production and manufacture comprises the sequential steps of (i) providing cells in growth media, and (ii) static infection of the cells of step (i) with media comprising a flavivirus at a low MOI. Preferably, the growth media of step (i) does not comprise a non-ionic surfactant. In this context, the cells can be Vero cells. In this context, the flavivirus can be a dengue virus. In this context, the vaccine can be a live, attenuated viral vaccine.

According to an embodiment of the invention, the method for large-scale flaviviral vaccine production and manufacture comprises the sequential steps of (i) providing cells that comprise abundant flaviviral receptors in growth media, and (ii) static infection of the cells of step (i) with media comprising a flavivirus at a low MOI. Preferably, the growth media of step (i) does not comprise a non-ionic surfactant. In this context, the cells can be

Vero cells. In this context, the flavivirus can be a dengue virus. In this context, the vaccine can be a live, attenuated viral vaccine.

Another infection parameter within the meaning of the method of infection is infecting the cells in suspension or in monolayer. The term “suspension” refers to cells that are dispersed in media. In the present context, the cells can be adherent cells that have been dissociated from the culture vessel using a dissociation reagent. Typically, for the purpose of large-scale vaccine production, it is understood that the cells should be infected immediately after using the dissociation agent (when they are still in suspension) to ensure infection efficiency.

It has been surprisingly found that infection of cells in monolayer can be used for large-scale flaviviral vaccine production and manufacture. Even more surprisingly, it has been found that infection of cells in monolayer can be used for large-scale attenuated flaviviral vaccine production and manufacture. In this context, the cells can be Vero cells. In this context, the flavivirus can be a dengue virus.

Furthermore, it has been surprisingly found that infection of cells in monolayer in combination with low MOI, provides for high infection efficiency for large-scale flaviviral vaccine production and even more surprisingly for large-scale attenuated flaviviral vaccine production. In this context, the cells can be Vero cells. In this context, the flavivirus can be a dengue virus.

Furthermore, it has been surprisingly found that infection of cells in monolayer in combination with low MOI and static infection, provides for high infection efficiency for large-scale flaviviral vaccine production and even more surprisingly for large-scale attenuated flaviviral vaccine production. In this context, the cells can be Vero cells. In this context, the flavivirus can be a dengue virus.

As used herein, the term “monolayer” refers to a layer of cells in which no, or substantially no cell is growing on top of another, but all are growing side by side and are often touching each other on the same growth surface.

According to an embodiment of the invention, the method for large-scale flaviviral vaccine production and manufacture comprises the sequential steps of (i) providing cells in growth media, and (ii) static infection of the cells of step (i) in monolayer with media comprising a flavivirus at a low MOI. Preferably, the growth media of step (i) does not comprise a non-ionic surfactant. In this context, the cells can be Vero cells. In this context, the flavivirus can be a dengue virus. In this context, the vaccine can be a live, attenuated viral vaccine.

Thus, infection of cells in monolayer can be used for large-scale flaviviral vaccine production and manufacture in a cell line that comprises abundant flaviviral receptors. Furthermore, infection of cells in monolayer in combination with low MOI can be used for large-scale flaviviral vaccine production and manufacture with high infection efficiency and/or high viral titers in a cell line that comprises abundant flaviviral receptors. Furthermore, infection of cells in monolayer in combination with low MOI and static infection can be used for large-scale flaviviral vaccine production and manufacture for high infection efficiency and/or high viral titers in a cell line that comprises abundant flaviviral receptors.

According to an embodiment of the invention, the method for large-scale flaviviral vaccine production and manufacture comprises the sequential steps of (i) providing cells that comprise abundant flaviviral receptors in growth media, and (ii) static infection of the cells of step (i) in monolayer with media comprising a flavivirus at a low MOI. Preferably, the growth media of step (i) does not comprise a non-ionic surfactant. In this context, the cells can be Vero cells. In this context, the flavivirus can be a dengue virus. In this context, the vaccine can be a live, attenuated viral vaccine.

Another infection parameter is the duration of infection and temperature of infection. The duration of infection can be within the range of 50 to 250 minutes, 60 to 200 minutes, 70 to 150 minutes, 70, 80, 90, 100, 110, 120, 130, 140 or 150 minutes. The incubation of the cells during infection can be within the temperature range between 36 to 39° C., preferably less than 39° C.

After infection, the cells may or may not be washed. If the cells are washed, the wash may be carried once, twice, thrice or four times. The wash can be conducted using any of the wash buffers such as phosphate-buffered saline (PBS), Dulbeco's phosphate-buffered saline (DPBS) or Tris buffered saline (TBS).

Accordingly, an aspect of the method for large-scale production and manufacture of flaviviral vaccines comprises a step of infection of cells with media comprising a flavivirus at a low MOI. In the context of the invention, it is to be understood that each of the individual method steps and/or components of ingredients described herein above are contemplated alone or in combination with each other. Thus, each possibility described herein above alone and in combination with each other represents a separate embodiment of the invention.

### Harvesting to Obtain a Harvest

After the infection of cells, a harvesting step can be carried out to obtain a harvest. Thus, according to an embodiment of the invention, the method for large-scale flaviviral vaccine production and manufacture comprises the sequential steps of (i) providing cells in growth media, (ii) infecting cells of step (i) with media comprising a virus at a low MOI, and (iii) harvesting to obtain a harvest. Preferably, the growth media of step (i) does not comprise a non-ionic surfactant. In this context, the cells can be Vero cells. In this context, the flavivirus can be a dengue virus. In this context, the vaccine can be a live, attenuated viral vaccine. In a preferred embodiment, the flaviviral vaccine is a tetravalent dengue vaccine including all four live, attenuated dengue serotypes including dengue serotype 1 such as dengue 2/1 chimera or TDV-1 represented by SEQ ID NO: 1 and/or SEQ ID NO:2, dengue serotype 2 or TDV-2 represented by SEQ ID NO: 3 and/or SEQ ID NO:4, dengue serotype 3 such as dengue serotype 2/3 or TDV-3 represented by SEQ ID NO: 5 and/or SEQ ID NO: 6, dengue serotype 4 such as dengue serotype 2/4 or TDV-4 represented by SEQ ID NO: 7 and/or SEQ ID NO:8. The methods described in this section are performed for each of the four serotypes separately.

The term “harvest” as used herein refers to the composition obtained from harvesting, e.g., from collecting the supernatant. Harvest refers to any intermediate composition from the time of collecting the supernatant up until the first processing step.

According to the invention, “harvesting” refers to an action of collecting at least a portion of the media from the production culture in which the virus is being produced and released. The “portion” of the media as used herein refers to the supernatant. Thus, “harvesting” refers to collecting the supernatant from the infected cells. In the context of the invention, “collecting the supernatant” or “a harvesting step” is immediately followed by adding fresh media such as e.g., growth media, such as described herein above. In a preferred embodiment, fresh media refers to growth media as described herein above.

In the context of the invention, harvesting can comprise a first harvesting step and at least one further harvesting step.

According to certain embodiments of the invention, 12-30 hours, 24 hours, 48 hours, 96 hours, or 120 hours, before the “first harvesting step”, a media change is carried out comprising collecting the supernatant, discarding the collected supernatant and adding fresh media. Preferably, the media change is carried out 12-30 hours before the first harvesting step. Thus, according to an embodiment of the invention, harvesting comprises a first harvesting step and at least one further harvesting step. In the context of the invention, the interval between each harvesting step is preferably at least 20-30 hours, 21-27 hours, 24 hours, 48 hours, or 96 hours.

Thus, according to an embodiment of the invention, the method for large-scale flaviviral vaccine production and manufacture comprises the sequential steps of (i) providing cells in growth media, (ii) infecting cells of step (i) with media comprising a virus at a low MOI, and (iii) harvesting to obtain a harvest, wherein harvesting comprises a first harvesting step and at least one further harvesting step and wherein at least 12-30 hours prior to the first harvesting step, a media change is carried out. Preferably, the growth media of step (i) does not comprise a non-ionic surfactant. In this context, the cells can be Vero cells. In this context, the flavivirus can be a dengue virus. In this context, the vaccine can be a live, attenuated viral vaccine.

According to an embodiment of the method of the invention, a media change is carried out 12-30 hours, 24 hours, 48 hours, 96 hours, or 120 hours prior to the first harvesting step, i.e., a media change is carried out on day 1, 2, 3, 4, 5, 6, 7, 8, 9, or 10 post infection and the first harvesting step is conducted on day 1, day 2, day 3, or day 4 post media change or day 2, day 3, day 4, day 5, day 6, day 7, day 8, day 9, day 10, day 11, day 12, day 13, or day 14 post infection followed by one, two, three, four, five, six, seven, eight or nine further harvesting steps. Each harvesting step is immediately followed by adding fresh media to the cells. The interval between each harvesting step is at least 20-30 hours, 21-27 hours, 24 hours, 48 hours, or 96 hours.

Thus according to an embodiment of the method of the invention, the first harvesting step is conducted on day 2 post infection followed by one further harvesting step on day 3 post infection, or the first harvesting step is conducted on day 3 post infection followed by one further harvesting step on day 4 post infection, or the first harvesting step is conducted on day 4 post infection followed by one further harvesting step on day 5 post infection, or the first harvesting step is conducted on day 5 post infection followed by one further harvesting step on day 6 post infection, or the first harvesting step is conducted on day 6 post infection followed by one further harvesting step on day 7 post infection. In this context, 12-30 hours before the first harvesting step, a media change is carried out. The first harvesting step is immediately followed by adding fresh media to the cells.

Thus according to an embodiment of the method of the invention, the first harvesting step is conducted on day 2 post infection followed by two further harvesting steps on day 3 and day 4 post infection, or the first harvesting step is conducted on day 3 post infection followed by two further harvesting steps on day 4 and day 5 post infection, or the first harvesting step is conducted on day 4 post infection followed by two further harvesting steps on day 5 and day 6 post infection, or the first harvesting step is conducted on day 5 post infection followed by two further harvesting steps on day 6 and day 7 post infection, or the first harvesting step is conducted on day 6 post infection followed by two further harvesting steps on day 7 and day 8 post infection. In this context, 12-30 hours before the first harvesting step, a media change is carried out. Each harvesting step is immediately followed by adding fresh media to the cells.

Thus according to an embodiment of the method of the invention, the first harvesting step is conducted on day 2 post infection followed by three further harvesting steps on day 3, day 4 and day 5 post infection, or the first harvesting step is conducted on day 3 post infection followed by three further harvesting steps on day 4, day 5, and day 6 post infection, or the first harvesting step is conducted on day 4 post infection followed by three further harvesting steps on day 5, day 6 and day 7 post infection, or the first harvesting step is conducted on day 5 post infection followed by three further harvesting steps on day 6, day 7, and day 8 post infection, or the first harvesting step is conducted on day 6 post infection followed by three further harvesting steps on day 7, day 8 and day 9 post infection. In this context, 12-30 hours before the first harvesting step, a media change is carried out. Each harvesting step is immediately followed by adding fresh media to the cells.

Thus according to an embodiment of the method of the invention, the first harvesting step is conducted on day 2 post infection followed by four further harvesting steps on day 3, day 4, day 5, and day 6 post infection, or the first harvesting step is conducted on day 3 post infection followed by four further harvesting steps on day 4, day 5, day 6, and day 7 post infection, or the first harvesting step is conducted on day 4 post infection followed by four further harvesting steps on day 5, day 6, day 7 and day 8 post infection, or the first harvesting step is conducted on day 5 post infection followed by four further harvesting steps on day 6, day 7, day 8, and day 9 post infection, or the first harvesting step is conducted on day 6 post infection followed by four further harvesting steps on day 7, day 8, day 9, and day 10 post infection. In this context, 12-30 hours before the first harvesting step, a media change is carried out. Each harvesting step is immediately followed by adding fresh media to the cells.

Thus according to an embodiment of the method of the invention, the first harvesting step is conducted on day 2 post infection followed by five further harvesting steps on day 3, day 4, day 5, day 6 and day 7 post infection, or the first harvesting step is conducted on day 3 post infection followed by five further harvesting steps on day 4, day 5, day 6, day 7, and day 8 post infection, or the first harvesting step is conducted on day 4 post infection followed by five further harvesting steps on day 5, day 6, day 7, day 8, and day 9 post infection. In a preferred embodiment, the first harvesting step is conducted on day 5 post infection followed by five further harvesting steps on day 6, day 7, day 8, day 9, and day 10 post infection. In one embodiment, the first harvesting step is conducted on day 6 post infection followed by five further harvesting steps on day 7, day 8, day 9, day 10, and day 11 post infection. In this context, 12-30 hours before the first harvesting step, a media change is carried out. Each harvesting step is immediately followed by adding fresh media to the cells.

Thus according to an embodiment of the method of the invention, the first harvesting step is conducted on day 2 post infection followed by six further harvesting steps on day 3, day 4, day 5, day 6, day 7 and day 8 post infection, or the first harvesting step is conducted on day 3 post infection followed by six further harvesting steps on day 4, day 5, day 6, day 7, day 8 and day 9 post infection, or the first harvesting step is conducted on day 4 post infection followed by six further harvesting steps on day 5, day 6, day 7, day 8, day 9 and day 10 post infection, or the first harvesting step is conducted on day 5 post infection followed by six further harvesting steps on day 6, day 7, day 8, day 9, day 10 and day 11 post infection, or the first harvesting step is conducted on day 6 post infection followed by six further harvesting steps on day 7, day 8, day 9, day 10, day 11 and day 12 post infection. In this context, 12-30 hours before the first harvesting step, a media change is carried out. Each harvesting step is immediately followed by adding fresh media to the cells.

Thus according to an embodiment of the method of the invention, the first harvesting step is conducted on day 2 post infection followed by seven further harvesting steps on day 3, day 4, day 5, day 6, day 7, day 8, and day 9 post infection, or the first harvesting step is conducted on day 3 post infection followed by seven further harvesting steps on day 4, day 5, day 6, day 7, day 8, day 9, and day 10 post infection, or the first harvesting step is conducted on day 4 post infection followed by seven further harvesting steps on day 5, day 6, day 7, day 8, day 9, day 10, and day 11 post infection, or the first harvesting step is conducted on day 5 post infection followed by seven further harvesting steps on day 6, day 7, day 8, day 9, day 10, day 11, and day 12 post infection, or the first harvesting step is conducted on day 6 post infection followed by seven further harvesting steps on day 7, day 8, day 9, day 10, day 11, day 12, and day 13 post infection. In this context, 12-30 hours before the first harvesting step, a media change is carried out. Each harvesting step is immediately followed by adding fresh media to the cells.

Thus according to an embodiment of the method of the invention, the first harvesting step is conducted on day 2 post infection followed by eight further harvesting steps on day 3, day 4, day 5, day 6, day 7, day 8, day 9, and day 10 post infection, or the first harvesting step is conducted on day 3 post infection followed by eight further harvesting steps on day 4, day 5, day 6, day 7, day 8, day 9, day 10, and day 11 post infection, or the first harvesting step is conducted on day 4 post infection followed by eight further harvesting steps on day 5, day 6, day 7, day 8, day 9, day 10, day 11, and day 12 post infection, or the first harvesting step is conducted on day 5 post infection followed by eight further harvesting steps on day 6, day 7, day 8, day 9, day 10, day 11, day 12 and day 13 post infection, or the first harvesting step is conducted on day 6 post infection followed by eight further harvesting steps on day 7, day 8, day 9, day 10, day 11, day 12, day 13 and day 14 post infection. In this context, 12-30 hours before the first harvesting step, a media change is carried out. Each harvesting step is immediately followed by adding fresh media to the cells.

Thus according to an embodiment of the method of the invention, the first harvesting step is conducted on day 2 post infection followed by nine further harvesting steps on day 3, day 4, day 5, day 6, day 7, day 8, day 9, day 10 and day 11 post infection, or the first harvesting step is conducted on day 3 post infection followed by nine further harvesting steps on day 4, day 5, day 6, day 7, day 8, day 9, day 10, day 11 and day 12 post infection, or the first harvesting step is conducted on day 4 post infection followed by nine further harvesting steps on day 5, day 6, day 7, day 8, day 9, day 10, day 11, day 12 and day 13 post infection, or the first harvesting step is conducted on day 5 post infection followed by nine further harvesting steps on day 6, day 7, day 8, day 9, day 10, day 11, day 12 day 13 and day 14 post infection, or the first harvesting step is conducted on day 6 post infection followed by nine further harvesting steps on day 7, day 8, day 9, day 10, day 11, day 12, day 13, day 14, and day 15 post infection. In this context, 12-30 hours before the first harvesting step, a media change is carried out. Each harvesting step is immediately followed by adding fresh media to the cells.

According to an embodiment of the invention, the large-scale production and manufacture of flaviviral vaccine may involve a step of media change 12-30 hours prior to the first daily harvest. For large-scale vaccine production and manufacture, another challenge is to maintain a balance between high viral titres and getting rid of process-related impurities. This is because the steps that get rid of process-related impurities may also adversely affect viral titres. It was surprisingly found that a change of media 12-30 hours, i.e., less than 48 hours prior to the first daily harvest achieves an optimal balance of waste of viral titre on one hand but reduction of the concentration of impurities on the other.

As used herein, the term “process-related impurities” refers to any undesirable component in such as nucleotides, polynucleotides, non-target proteins (such as host cell proteins, HCP), other cellular components (such as lipids and glycolipids), and any other contaminants that arise from, or during, production, separation, and/or purification processes. Other process related impurities, specifically in the production of large-scale vaccines include defective interfering particles or DIPs. DIPs are a natural byproduct of replicating viruses. DIPs interfere with the propagation and spread of infectious standard virus (STV), reduce virus yields by competing for viral and cellular resources and induce antiviral responses. It has been surprisingly found that the method of the invention gets rid of process-related impurities, which significantly enhances viral production as seen by significant improvement in viral titres.

It has been surprisingly found that a media change 12-30 hours, i.e., less than 48 hours prior to the first harvesting step in the method of flaviviral vaccine production and manufacture does not significantly impact the viral titres as compared to no media change. Further, it has been surprisingly found that a media change 12-30 hours prior to the first harvesting step in the method of flaviviral vaccine production and manufacture in combination with at least two, at least three, at least four, or most preferably, at least five further harvesting steps such as seven further harvesting steps or more does not significantly impact the viral titres as compared to no media change. In this context, the flavivirus can be a dengue virus. In this context, the vaccine can be a live, attenuated viral vaccine.

Thus, according to an embodiment of the method of the invention, for large-scale flaviviral vaccine production and manufacture, a media change 12-30 hours prior to the first harvesting step in combination with infection of cells at a low MOI provides for high viral titres. Further, according to an embodiment of the method of the invention, for large-scale flaviviral vaccine production and manufacture, media change 12-30 hours prior to the first harvesting step followed by at least three further harvesting steps such as five further harvesting steps such as seven further harvesting steps or more in combination with infection of cells at a low MOI range provides for high viral titres. In this context, the cells can be Vero cells. In this context, the flavivirus can be a dengue virus. In this context, the vaccine can be a live, attenuated viral vaccine.

Thus, according to an embodiment of the invention, the method for large-scale flaviviral vaccine production and manufacture comprises the sequential steps of (i) providing cells in growth media, (ii) infecting cells of step (i) with media comprising a virus at a low MOI, and (iii) harvesting to obtain a harvest, wherein harvesting comprises a first harvesting step and at least three further harvesting steps such as five further harvesting steps such as seven further harvesting steps or more and wherein 12-30 hours prior to the first harvesting step, a media change is carried out. Preferably, the growth media of step (i) does not comprise a non-ionic surfactant. In this context, the cells can be Vero cells. In this context, the flavivirus can be a dengue virus. In this context, the vaccine can be a live, attenuated viral vaccine.

Thus, for large-scale flaviviral vaccine production and manufacture, media change 12-30 hours prior to the first harvesting step in combination with static infection of cells at a low MOI range provides for high viral titres. Further, for large-scale flaviviral vaccine production and manufacture, media change 12-30 hours prior to the first harvesting step followed by at least three further harvesting steps such as five further harvesting steps such as seven further harvesting steps or more in combination with static infection of cells at a low MOI provides for high viral titres. In this context, the cells can be Vero cells. In this context, the flavivirus can be a dengue virus. In this context, the vaccine can be a live, attenuated viral vaccine.

Thus, according to an embodiment of the invention a large-scale flaviviral vaccine production and manufacture comprises the sequential steps of (i) providing cells in growth media, (ii) static infection of the cells of step (i) with media comprising a flavivirus at a low MOI, (iii) harvesting to obtain a harvest, wherein harvesting comprises a first harvesting step and at least three further harvesting steps such as five further harvesting steps such as seven further harvesting steps or more and wherein 12-30 hours prior to the first harvesting step, a media change is carried out. Preferably, the growth media of step (i) does not comprise a non-ionic surfactant. In this context, the cells can be Vero cells. In this context, the flavivirus can be a dengue virus. In this context, the vaccine can be a live, attenuated viral vaccine.

Thus, for large-scale flaviviral vaccine production and manufacture, media change 12-30 hours prior to the first harvesting step in combination with static infection of cells in a monolayer at a low MOI provides for high viral titres. Further, for large-scale flaviviral vaccine production and manufacture, media change 12-30 hours prior to the first harvesting step followed by at least three further harvesting steps such as five further harvesting steps such as seven further harvesting steps or more in combination with static infection of cells in a monolayer at a low MOI provides for high flaviviral titres. In this context, the cells can be Vero cells. In this context, the flavivirus can be a dengue virus. In this context, the vaccine can be a live, attenuated viral vaccine.

Thus, according to an embodiment of the invention a large-scale flaviviral vaccine production and manufacture comprises the sequential steps of (i) providing cells in growth media, (ii) static infection of the cells of step (i) in monolayer with media comprising a flavivirus at a low MOI, (iii) harvesting to obtain a harvest, wherein harvesting comprises a first harvesting step and at least three further harvesting steps such as five further harvesting steps such as seven further harvesting steps or more and wherein 12-30 hours prior to the first harvesting step, a media change is carried out. Preferably, the growth media of step (i) does not comprise a non-ionic surfactant. In this context, the cells can be Vero cells. In this context, the flavivirus can be a dengue virus. In this context, the vaccine can be a live, attenuated viral vaccine.

Thus, according to an embodiment of the method of the invention, for large-scale flaviviral vaccine production and manufacture, media change 12-30 hours prior to the first harvesting step in combination with infection of cells that comprise abundant flaviviral receptors at a low MOI range provides for high viral titres. Further, according to the method of the invention, for large-scale flaviviral vaccine production and manufacture, media change 12-30 hours prior to the first harvesting step followed by at least three further harvesting steps such as five further harvesting steps such as seven further harvesting steps or more in combination with infection of cells that comprise abundant flaviviral receptors at a low MOI range provides for high viral titres. In this context, the cells can be Vero cells. In this context, the flavivirus can be a dengue virus. In this context, the vaccine can be a live, attenuated viral vaccine.

Thus, according to an embodiment of the invention a large-scale flaviviral vaccine production and manufacture comprises the sequential steps of (i) providing cells that comprise abundant flaviviral receptors in growth media, (ii) infecting the cells of step (i) with media comprising a flavivirus at a low MOI, (iii) harvesting to obtain a harvest, wherein harvesting comprises a first harvesting step and at least three further harvesting steps such as five further harvesting steps such as seven further harvesting steps or more and wherein 12-30 hours prior to the first harvesting step, a media change is carried out. Preferably, the growth media of step (i) does not comprise a non-ionic surfactant. In this context, the cells can be Vero cells. In this context, the flavivirus can be a dengue virus. In this context, the vaccine can be a live, attenuated viral vaccine.

Thus, according to an embodiment of the method of the invention, for large-scale flaviviral vaccine production and manufacture, media change 12-30 hours prior to the first harvesting step in combination with static infection of cells that comprise abundant flaviviral receptors at a low MOI range provides for high flaviviral titres. Further, according to an embodiment of the method of the invention, for large-scale flaviviral vaccine production and manufacture, media change 12-30 hours prior to the first harvesting step followed by at least three further harvesting steps such as five further harvesting steps such as seven further harvesting steps or more in combination with static infection of cells that comprise abundant flaviviral receptors at a low MOI range provides for high flaviviral titres. In this context, the cells can be Vero cells. In this context, the flavivirus can be a dengue virus. In this context, the vaccine can be a live, attenuated viral vaccine.

Thus, according to an embodiment of the method of the invention, for large-scale flaviviral vaccine production and manufacture, media change 12-30 hours prior to the first harvesting step in combination with static infection of cells in monolayer that comprise abundant flaviviral receptors at a low MOI range provides for high flaviviral titres. Further, according to an embodiment of the method of the invention, for large-scale flaviviral vaccine production and manufacture, media change 12-30 hours prior to the first harvesting step followed by at least three further harvesting steps such as five further harvesting steps such as seven further harvesting steps or more in combination with static infection of cells in monolayer that comprise abundant flaviviral receptors at a low MOI range provides for high flaviviral titres. In this context, the cells can be Vero cells. In this context, the flavivirus can be a dengue virus. In this context, the vaccine can be a live, attenuated viral vaccine.

Thus, according to an embodiment of the invention a large-scale flaviviral vaccine production and manufacture comprises the sequential steps of (i) providing cells that comprise abundant flaviviral receptors in growth media, (ii) static infection of cells of step (i) in monolayer with media comprising a flavivirus at a low MOI, (iii) harvesting to obtain a harvest, wherein harvesting comprises a first harvesting step and at least three further harvesting steps such as five further harvesting steps such as seven further harvesting steps or more and wherein 12-30 hours prior to the first harvesting step, a media change is carried out. Preferably, the growth media of step (i) does not comprise a non-ionic surfactant. In this context, the cells can be Vero cells. In this context, the flavivirus can be a dengue virus. In this context, the vaccine can be a live, attenuated viral vaccine.

In certain embodiments, the pH throughout the steps mentioned above is maintained in a range from 7.6 to 8.1.

Accordingly, an embodiment of the method for large-scale production and manufacture of flaviviral vaccines, comprises a step of harvesting comprising a first harvesting step and at least one further harvesting step, wherein a media change is carried out at least 12-30 hours prior to the first harvesting step. In the context of the invention, it is to be understood that each of the individual method steps and/or components of ingredients described herein above are contemplated alone or in combination with each other. Thus, each possibility described herein above alone and in combination with each other represents a separate embodiment of the invention.

### Processing the Harvest

According to an embodiment of the invention, the method for large-scale flaviviral vaccine production and manufacture comprises the sequential steps of (i) providing cells in growth media, (ii) infecting the cells of step (i) with media comprising a flavivirus at a low MOI, (iii) harvesting to obtain a harvest and (iv) processing the harvest of step (iii). In this context, the cells can be Vero cells. In this context, the flavivirus can be a dengue virus. In this context, the vaccine can be a live, attenuated viral vaccine. In a preferred embodiment, the flaviviral vaccine is a tetravalent dengue vaccine including all four live, attenuated dengue serotypes including dengue serotype 1 such as dengue 2/1 chimera or TDV-1 represented by SEQ ID NO: 1 and/or SEQ ID NO:2, dengue serotype 2 or TDV-2 represented by SEQ ID NO: 3 and/or SEQ ID NO:4, dengue serotype 3 such as dengue serotype 2/3 or TDV-3 represented by SEQ ID NO: 5 and/or SEQ ID NO:6, dengue serotype 4 such as dengue serotype 2/4 or TDV-4 represented by SEQ ID NO: 7 and/or SEQ ID NO:8. The methods described in this section are performed for each of the four serotypes separately.

As discussed above, the term “harvest” as used herein refers to the composition obtained from the action of harvesting, e.g., from collecting the supernatant. “Harvest” or “crude harvest” are used interchangeably and refer to any intermediate composition from the time of collecting the supernatant up until the first processing step.

The term “processing the harvest” refers to one or more operational steps conducted on the obtained harvest. Thus, processing the harvest may comprise one or all of the steps including clarification, stabilization, freezing, thawing and pooling. In a preferred embodiment, processing the harvest refers to clarification and stabilization in that order. In a preferred embodiment, processed harvest refers to clarified and stabilized harvest in that order.

### Processing the Harvest: Clarification of the Harvest

In the context of the invention, processing the harvest may comprise clarification of the harvest. The harvest can be clarified to get rid of cells and cell debris that may have accumulated during the infection process. The clarification of the harvest may comprise centrifugation, and/or one or more non-membrane filtration steps such as depth filtration. These steps are performed on the harvest.

Depth filtration uses a porous filtration medium to separate particles and solids from a liquid. The filter media which may be used in depth filtration can be cellulose acetate, polypropylene, cellulose acetate protected by glassfiber fleece and polyethersulfone. The filter used in depth filtration can have a pore size of e.g., 0.1 μm to 1 μm, 0.2 μm to 0.8 μm, or 0.2 μm to 0.45 μm.

The filters used in the clarification of the harvest can be heterogenous double layer of polyethersulfone with pore sizes of 0.2 μm and 0.45 μm. Thus, for the purpose of clarification of the harvest, several heterogenous double layer filters can be used such as Sartoclean (Cellulose Acetate) 0.8+0.65 μm, Sartopure (Polyporpylene) 0.65 μm, Sartoclean (Glassfiber Fleeces) 0.8+0.65 μm, Sartopore 2 (Polyethersulfone) 0.8+0.45 μm, Sartopore 2 (Polyethersulfone) 0.45+0.2 μm, or Sartopore 2 XLG (Polyethersulfone) 0.8+0.2 μm. Each possibility represents a separate embodiment of the invention.

At the end of the clarification step, the composition obtained is termed “clarified harvest”, which refers to a harvest that has been subjected to the processing step of clarification.

Thus, according to an embodiment of the invention, the method for large-scale flaviviral vaccine production and manufacture comprises the sequential steps of (i) providing cells in growth media, (ii) infecting the cells of step (i) with media comprising a flavivirus at a low MOI, (iii) harvesting to obtain a harvest and (iv) processing the harvest of step (iii) comprising clarification of the harvest. In this context, the cells can be Vero cells. In this context, the flavivirus can be a dengue virus. In this context, the vaccine can be a live, attenuated viral vaccine.

### Processing the Harvest: Stabilization of the Harvest

In the context of the invention, processing the harvest may comprise stabilization. For the method of the invention, a buffered excipient composition comprising one or more of a surfactant, a sugar and a protein can be used. The buffered excipient composition may comprise a TRIS buffer, phosphate buffer, a sodium citrate buffer, a 2-(N-morpholino) ethanesulfonic acid (MES) buffer, a 3-morpholinopropane-1-sulfonic acid (MOPS) buffer, and a salt (e.g., sodium chloride, magnesium chloride, or calcium chloride). In some embodiments, the buffer may be free of proteins. In one embodiment, the buffered excipient composition comprises a non-ionic surfactant, a sugar and a protein in phosphate buffer saline (PBS). The buffered excipient composition is also termed stabilization buffer. Thus, the terms buffered excipient composition and stabilization buffer are used interchangeably.

Thus, for the method of the invention, a stabilization buffer comprising one or more excipients including a surfactant, a sugar and a protein can be used. The stabilization buffer may comprise a phosphate buffer, a sodium citrate buffer, a 2-(N-morpholino) ethanesulfonic acid (MES) buffer, a 3-morpholinopropane-1-sulfonic acid (MOPS) buffer, and a salt (e.g., sodium chloride, magnesium chloride, or calcium chloride). In some embodiments, the stabilization buffer may be free of proteins. In one embodiment, the stabilization buffer comprises a non-ionic surfactant, a sugar and a protein in phosphate buffer saline (PBS).

As used herein, the term “sugar” includes monosaccharides, (e.g. glucose, galactose, ribose, mannose, rhamnose, talose, xylose, or allose arabinose.), disaccharides (e.g. trehalose, sucrose, maltose, isomaltose, cellibiose, gentiobiose, laminaribose, xylobiose, mannobiose, lactose, or fructose), trisaccharides (e.g. acarbose, raffinose, melizitose, panose, or cellotriose) and sugar polymers (e.g. dextran, xanthan, pullulan, cyclodextrins, amylose, amylopectin, starch, cello-oligosaccharides, cellulose, maltooligosaccharides, glycogen, chitosan, or chitin). In addition, the term “sugar” as used herein also includes sugar alcohols such as mannitol, sorbitol, arabitol, erythritol, maltitol, xylitol, glycitol, glycol, polyglycitol, polyethylene glycol, polypropylene glycol, and glycerol.

The sugar used for the stabilization buffer can be a non-reducing sugar. Non-reducing sugars do not contain an aldehyde or ketone group which is capable of being oxidized. Examples of non-reducing sugars include sucrose, trehalose or its hydrates such as trehalose dihydrate.

The concentration of the sugar in the stabilization buffer can be 20% to 60% (w/v), 25% to 55% (w/v), or 35% to 50%, or 45% (w/v). Preferably, 35% to 50% (w/v). The concentration of non-reducing sugar in the stabilization buffer can be 20% to 60% (w/v), 25% to 55% (w/v), or 35% to 50%, or 45% (w/v). Preferably, 35% to 50% (w/v). The concentration of sucrose in the stabilization buffer can be 20% to 60% (w/v), 25% to 55% (w/v), or 35% to 50%, or 45% (w/v). Preferably, 35% to 50% (w/v). The concentration of trehalose in the stabilization buffer can be 20% to 60% (w/v), 25% to 55% (w/v), or 35% to 50%, or 45% (w/v). Preferably, 35% to 50% (w/v). The concentration of trehalose dihydrate in the stabilization buffer can be 20% to 60% (w/v), 25% to 55% (w/v), or 35% to 50%, or 45% (w/v). Preferably, 35% to 50% (w/v).

For carrying out an embodiment of the method of the invention, the surfactant in the stabilization buffer can be a non-ionic surfactant. The concentration of the non-ionic surfactant in the stabilization buffer can be preferably between 0.25% to 5%. For example, 1.5%, 1.75%, 2%, 2.25%, 2.5%, 2.75% or 3%. Each possibility represents a separate embodiment of the invention. For example, the concentration of poloxamer 407 and/or F127 can be preferably between 0.25% to 5%. For example, 1.5%, 1.75%, 2%, 2.25%, 2.5%, 2.75% or 3%. Each possibility represents a separate embodiment of the invention. For example, the concentration of poloxamer 403 and/or F123 can be preferably between 0.25% to 5%. For example, 1.5%, 1.75%, 2%, 2.25%, 2.5%, 2.75% or 3%. Each possibility represents a separate embodiment of the invention. For example, the concentration of P123 can be preferably between 0.25% to 5%. For example, 1.5%, 1.75%, 2%, 2.25%, 2.5%, 2.75% or 3%. Each possibility represents a separate embodiment of the invention. For example, the concentration of P85 can be preferably between 0.25% to 5%. For example, 1.5%, 1.75%, 2%, 2.25%, 2.5%, 2.75% or 3%. Each possibility represents a separate embodiment of the invention.

The protein which may be present in the stabilization buffer can be any protein which is essentially inert and does not react with the virus. In particular, the protein does not affect the structure or infectivity of the virus. Thus, the protein can be a structural protein or a serum protein. The protein can be selected from an albumin, collagen, hydrolyzed collagen, gelatin and hydrolyzed gelatin.

The albumins are a family of globular non-glycosylated proteins which are inter alia present in the blood of a vertebrate. They are water-soluble, moderately soluble in concentrated salt solutions, and experience heat denaturation. Suitable albumins for use in the method of the present invention include mammalian serum albumins such as human serum albumin and bovine serum albumin or lactalbumin. Serum albumin is one of the most common proteins in vertebrate blood and has multiple functions. Human serum albumin is not glycosylated and has a single free thiol group. The human serum albumin may be recombinant human serum albumin, or it may be human serum albumin purified from human serum. Preferably, human serum albumin purified from human serum is used.

As used herein, the term “collagen” is used in reference to the extracellular family of fibrous proteins that are characterized by their stiff, triple-stranded helical structure. Three collagen polypeptide chains (“α-chains”) are wound around each other to form this helical molecule. The term is also intended to encompass the various types of collagen, although the preferred form is type I collagen.

The term “gelatin” refers to a heterogeneous mixture of water-soluble proteins of high average molecular weight. Gelatin is not found in nature but derived from collagen by hydrolytic action. Gelatin is obtained by boiling skin, tendon, bones, ligaments in water. Gelatin is colorless or slightly yellowish, transparent, sheets, flakes or coarse powder which absorbs in a range between about 5 times and about 10 times its weight of water to form a gel in solutions.

The concentration of the protein in the stabilization buffer can be 0.1% to 0.5% (w/v), 0.2% to 0.4% (w/v), 0.15% to 0.3% (w/v) or 0.3% (w/v), preferably 0.2% to 0.4% (w/v). The concentration of the albumin in the stabilization buffer can be 0.1% to 0.5% (w/v), 0.2% to 0.4% (w/v), 0.15% to 0.3% (w/v) or 0.3% (w/v), preferably 0.2% to 0.4% (w/v). The concentration of human serum albumin or human serum albumin purified from human serum in the stabilization buffer can be 0.1% to 0.5% (w/v), 0.2% to 0.4% (w/v), 0.15% to 0.3% (w/v) or 0.3% (w/v), preferably 0.2% to 0.4% (w/v). The concentration of collagen in the stabilization buffer can be 0.1% to 0.5% (w/v), 0.2% to 0.4% (w/v), 0.15% to 0.3% (w/v) or 0.3% (w/v), preferably 0.2% to 0.4% (w/v). The concentration of gelatin in the stabilization buffer can be 0.1% to 0.5% (w/v), 0.2% to 0.4% (w/v), 0.15% to 0.3% (w/v) or 0.3% (w/v), preferably 0.2% to 0.4% (w/v).

The concentration of the excipients of the stabilization buffer in the stabilized harvest is between 1/7^{th} to 1/15^{th} times the concentration of the excipients in the stabilization buffer, preferably 1/12^{th} to 1/15^{th} times. For example, if the concentration of the excipients of the stabilization buffer in the stabilized harvest is 1/14^{th} times the concentration of the excipients in the stabilization buffer, the following exemplary concentrations in the stabilized harvest would result.

The concentration of the sugar in the stabilized harvest can be e.g., 1.4% to 4% (w/v), 1.8% to 3.9% (w/v), 2.5% to 3.6% (w/v) or 3% (w/v). The concentration of non-reducing sugar in the stabilized harvest can be 1.4% to 4% (w/v), 1.8% to 3.9% (w/v), 2.5% to 3.6% (w/v) or 3% (w/v). The concentration of sucrose in the stabilized harvest can be 1.4% to 4% (w/v), 1.8% to 3.9% (w/v), 2.5% to 3.6% (w/v) or 3% (w/v). The concentration of trehalose in the stabilized harvest can be 1.4% to 4% (w/v), 1.8% to 3.9% (w/v), 2.5% to 3.6% (w/v) or 3% (w/v) The concentration of trehalose dihydrate in the stabilized harvest can be 1.4% to 4% (w/v), 1.8% to 3.9% (w/v), 2.5% to 3.6% (w/v) or 3% (w/v)

For carrying out an embodiment of the method of the invention, the non-ionic surfactant in the stabilized harvest can be between 0.01% to 0.4%. For example, 0.1%, 0.125%, 0.14%, 0.16%. 0.18%, or 0.2%. Each possibility represents a separate embodiment of the invention. For example, the concentration of poloxamer 407 and/or F127 can be between 0.01% to 0.4%. For example, 0.1%, 0.125%, 0.14%, 0.16%. 0.18%, or 0.2%. Each possibility represents a separate embodiment of the invention. For example, the concentration of poloxamer 403 and/or F123 can be between 0.01% to 0.4%. For example, 0.1%, 0.125%, 0.14%, 0.16%. 0.18%, or 0.2%. Each possibility represents a separate embodiment of the invention. For example, the concentration of P123 can be between 0.01% to 0.4%. For example, 0.1%, 0.125%, 0.14%, 0.16%. 0.18%, or 0.2%. Each possibility represents a separate embodiment of the invention. For example, the concentration of P85 can be between 0.01% to 0.4%. For example, 0.1%, 0.125%, 0.14%, 0.16%. 0.18%, or 0.2%. Each possibility represents a separate embodiment of the invention.

The concentration of the albumin in the stabilized harvest can be 0.007% to 0.04% (w/v), 0.01% to 0.03% (w/v), 0.01% to 0.075% (w/v) or 0.02% (w/v). The concentration of human serum albumin or human serum albumin purified from human serum in the stabilized harvest can be 0.007% to 0.04% (w/v), 0.01% to 0.03% (w/v), 0.01% to 0.075% (w/v) or 0.02% (w/v). The concentration of collagen in the stabilized harvest can be 0.007% to 0.04% (w/v), 0.01% to 0.03% (w/v), 0.01% to 0.075% (w/v) or 0.02% (w/v). The concentration of gelatin in the stabilized harvest can be 0.007% to 0.04% (w/v), 0.01% to 0.03% (w/v), 0.01% to 0.075% (w/v) or 0.02% (w/v).

For other ratios such as 1/7^{th} to 1/15^{th} or 1/12^{th} to 1/15th, a skilled person can easily calculate the resulting concentrations of the excipients in the stabilized harvest.

The composition termed “stabilized harvest”, refers to a crude harvest that has been subjected to the processing step of stabilization, or a crude harvest that has been subjected to the processing steps of clarification and stabilization in any order. Thus, the term clarified, and stabilized harvest refers to a crude harvest that has been clarified and stabilized in that order. Thus, the term stabilized and clarified harvest refers to a crude harvest that has been stabilized and clarified in that order.

Thus, according to an embodiment of the invention, the method for large-scale viral vaccine production and manufacture comprises the sequential steps of (i) providing cells in growth media, (ii) infecting the cells of step (i) with media comprising a flavivirus at a low MOI, iii) harvesting to obtain a harvest and (iv) processing the harvest of step (iii) comprising stabilization of the harvest. In this context, the cells can be Vero cells. In this context, the flavivirus can be a dengue virus. In this context, the vaccine can be a live, attenuated viral vaccine. Thus, according to an embodiment of the invention, the method for large-scale viral vaccine production and manufacture comprises the sequential steps of (i) providing cells in growth media, (ii) infecting the cells of step (i) with media comprising a flavivirus at a low MOI, (iii) harvesting to obtain a harvest and (iv) processing the harvest of step (iii) comprising clarification and stabilization of the harvest. In this context, the cells can be Vero cells. In this context, the flavivirus can be a dengue virus. In this context, the vaccine can be a live, attenuated viral vaccine. Thus, according to an embodiment of the invention, the method for large-scale viral vaccine production and manufacture comprises the sequential steps of (i) providing cells in growth media, (ii) infecting the cells of step (i) with media comprising a flavivirus at a low MOI, (iii) harvesting to obtain a harvest and (iv) processing the harvest of step (iii) comprising stabilization and clarification of the harvest. In this context, the cells can be Vero cells. In this context, the flavivirus can be a dengue virus. In this context, the vaccine can be a live, attenuated viral vaccine.

### Processing the Harvest: Freezing, Thawing and Pooling

Freezing is another way of processing and can be used to store and freeze a composition at any time during the processing at or below −30° C.

Thawing is another way of processing and refers to adapting the frozen composition to room temperature.

Pooling is another way of processing and comprises pooling one or more compositions at any time during the processing in order to carry out further processing steps on the pooled composition or purifying steps on the pooled processed harvest.

According to an embodiment of the invention, each “harvest” or “crude harvest” may be frozen immediately after the harvesting step followed by thawing and then may be subjected to one or several processing steps or purifying steps on the processed harvest. Furthermore, according to an embodiment of the method of the invention, a first harvest obtained from the first harvesting step may be immediately frozen after the harvesting step followed by thawing and pooling together with at least one further harvest obtained from at least one further harvesting step and then subjected to one or more further processing steps or purifying steps.

Furthermore, according to an embodiment of the method of the invention, a first harvest obtained from the first harvesting step can be immediately clarified and then immediately frozen followed by thawing and pooling with at least one further clarified harvest obtained from at least one further harvesting step (that may or may not have been frozen and thawed) and then subjected to one or more further processing steps or purifying steps.

Furthermore, according to an embodiment of the method of the invention, a first harvest obtained from the first harvesting step can be immediately clarified and stabilized then immediately frozen followed by thawing and pooling with at least one further clarified and stabilized harvest obtained from at least one further harvesting step (that may or may not have been frozen and thawed) and then subjected to one or more further processing steps or purifying steps.

In a preferred embodiment, a first harvest obtained from the first harvesting step is immediately clarified and stabilized on the same day.

“Immediately on the same day” or “on the same day” as used herein throughout the disclosure refers to a time interval ranging from a few minutes to a few hours but less than 20 hours preferably less than 10 hours, or less than 12 hours such as 1 hour or less, 2 hours or less, 3, hours or less, 4 hours or less or, 5 hours or less. 6 hours or less, 7 hours or less, 8 hours or less, 9 hours or less.

### Purifying the Processed Harvest: At Least One Chromatography Step

In a preferred embodiment, a first harvest obtained from the first harvesting step is immediately clarified, stabilized and purified with at least one chromatography step on the same day.

Purifying the processed harvest may comprise a step of removal of host cell DNA. The primary objective of this step is removal of the host cell DNA (HC DNA) while ensuring maximal virus recovery. The “host cell DNA” is derived from the cell in which the infectious viral particles were produced. The host cell DNA can be distinguished from the viral nucleic acids by its nucleic acid sequences which differ from those of the viral nucleic acids. Several methods are known in the art for removing HC DNA. These are either enzymatic methods or enzymatic methods in combination with chromatography-based methods.

In the context of the invention, an enzymatic method may be used for the removal of host cell DNA followed by one or more chromatography steps. One of the enzymatic methods is enzymatic degradation with endonucleases. These enzymes act on nucleic acid by specifically catalyzing the hydrolysis of internal phosphodiester bonds in DNA and RNA chains breaking them into smaller nucleotides. Smaller nucleotides/nucleic acid fragments and endonucleases can eventually be easily removed from the process during subsequent downstream processing.

In the context of the invention, purifying the processed harvest can necessarily comprise a chromatography step. Irrespective of the fact whether an enzymatic method for HC DNA removal is used or not, a chromatography step is preferably necessarily included and may be an ion exchange chromatography, (i.e., either a cation exchange chromatography, or an anion exchange chromatography).

For an embodiment of the method of the invention, the chromatography step for host cell DNA removal can be performed in bind-and-elute or in flow-through mode. In the bind-and-elute mode the substance to be purified binds to the anion exchange groups while the impurities do not bind. The substance to be purified can be eluted from the anion exchange groups by changing one or more chromatography conditions such as the salt condition in the buffer or the pH of the buffer. In the flow-through mode the impurities bind to the anion exchange groups while the substance to be purified does not bind but can be collected directly from the flow-through.

Ion exchange chromatography, (i.e., each of cation and anion exchange chromatography) relies on charge-charge interactions between components in a sample and the charges of the immobilized functional group. In anion exchange chromatography, the binding ions present in the sample are negative, and the immobilized functional group is positive while in cation exchange chromatography, the binding ions present in the sample are positive and the immobilized functional group is negative.

Commonly used anion exchange functional groups are Q-resin, a quaternary amine, and DEAE resin (diethylaminoethane). Commonly used cation exchange groups are CM, a quaternary amine, S, a methyl sulfonate, and SP, a sulphonyl group. However, in general the anion or cation exchange chromatography step can be performed with all common commercially available anion or cation exchange resins or membranes.

Typical strong anion exchange groups that can be used for the purpose of the invention comprise functional groups such as: quaternary aminoethyl (QAE) moieties, primary amine (PA), quaternary ammonium (Q) moieties and trimethylammoniumethyl (TMAE) groups. Resins having quaternary aminoethyl (QAE) moieties include, e.g., Toyopearl QAE (available from Tosoh Bioscience, Germany), Selectacel QAE (a quaternary aminoethyl derivative of cellulose, available from Polysciences Inc., Pennsylvania USA) and others. Resins having quaternary ammonium (Q) moieties include, e.g., Sartobind® Q (available from Sartorius, Germany), Mustang® Q Acrodisc (available from Pall, Germany), Q Sepharose XL, Q Sepharose FF, Q Sepharose HP, Resource Q (available from GE Healthcare, Germany), Macro Prep High Q (Bio-Rad, California, USA), Toyopearl Super Q (available from Tosoh Bioscience, Germany) and UNOsphere Q (available from Bio-Rad, California, USA). Resins having trimethylammoniumethyl (TMAE) groups include, e.g., Fractogel EMD TMAE (available from Merck, Germany). Resins having primary amine groups include Sartobind STIC primary amine resins (available from Sartorius, Germany).

For example, the anion exchange chromatography step can be performed with an anion exchange chromatography membrane having quaternary ammonium groups. The membrane base material can be selected from stabilized reinforced cellulose and polyethersulfone. The membrane base material can be stabilized reinforced cellulose and the functional group is a quaternary ammonium group. The anion exchange chromatography step may or may not involve the use of a monolithic support. The nominal pore size of the membrane can be 0.5 μm to 5 μm or greater than 3 μm. The membrane area can be 20 to 50 cm², 25 to 45 cm², 30 to 40 cm² such as, for example, 32 cm², 34 cm², 36 cm², or 38 cm². The flow rate can be 1 to 50 ml/min, or 10 to 40 ml/min depending on the column used.

For example, the cation exchange chromatography step can be performed with a cation exchange chromatography membrane having quaternary amine groups. The membrane base material can be selected from stabilized reinforced cellulose and polyethersulfone. The cation exchange chromatography step may or may not involve the use of a monolithic support. The nominal pore size of the membrane can be 0.5 μm to 5 μM or greater than 3 μm. The membrane area can be 20 to 50 cm², 25 to 45 cm², 30 to 40 cm². The flow rate can be 1 to 50 ml/min, or 10 to 40 ml/min.

In certain embodiments of the method of the present invention, host cell DNA is removed from a sample comprising viral particles and host cell DNA. The term “removing host cell DNA” means that the content of host cell DNA after the method of the invention has been performed is lower than the content of host cell DNA before the method of the invention is performed. In one embodiment, the method of the present invention results in an at least ten-fold reduction of the host cell DNA content. Preferably, the method of the present invention results in a reduction of the host cell DNA content by at least 12-fold or 15-fold, more preferably by at least 18-fold or 20-fold and most preferably by at least 22-fold. The fold reduction can be calculated by dividing the host cell DNA content in the sample before the method is performed with the host cell DNA content in the sample after the method has been performed. Preferably, the content of host cell DNA after the method of the invention has been performed is lower than the detection limit of the assay used for determining the host cell DNA content. In one embodiment, the content of host cell DNA after the method of the invention has been performed is 50 ng/ml or lower.

Methods to determine the host cell DNA content in a biological sample obtained from a host cell are known in the art and include quantitative PCR using primers which specifically bind to the host cell DNA, but not to the viral nucleic acids. Kits for determining host cell DNA content are commercially available for example from ThermoFisher. Preferably, the Picogreen® dye is used to determine the host cell DNA content.

Typically for large-scale vaccine production, the harvest, or the clarified harvest, or the clarified and stabilized harvest or the stabilized and clarified harvest, is first concentrated with an ultrafiltration step before loading it on the chromatography column such as hydrophobic interaction chromatography column, or ion exchange chromatography column, i.e., anion exchange chromatography column or cation exchange chromatography column. This is mainly because concentrating the harvest before column chromatography enables faster loading and increased column capacity for improved productivity. Moreover, typically, it is understood that ultrafiltration before a chromatography step enhances the efficiency of the chromatography. Furthermore, typically, it is understood that ultrafiltration of the harvest, or the clarified harvest, or the clarified and stabilized harvest, or the stabilized and clarified harvest before the chromatography step requires less dilution to adjust pH and conductivity for the chromatography step, which ultimately increases column productivity and reduces the use of consumables, contributing to improved process economics. “Concentrated” as used herein means that the total volume of the harvest is reduced, thus increasing the concentration of the ingredients in the harvest.

Ultrafiltration (UF) as used herein refers to techniques that rely on the use of polymeric membranes with highly defined pore sizes to separate molecules according to size. Simply put, UF procedures rely on the use of fluid pressure to drive the migration of the smaller molecules through a UF membrane with the simultaneous retention of larger molecules. Ultrafiltration can be performed in one of two operational modes: Direct Flow Filtration (DFF), or Tangential Flow Filtration (TFF).

It has been surprisingly found that a chromatography step can be implemented directly on the harvest, or the clarified harvest, or the clarified and stabilized harvest, or the stabilized and clarified harvest i.e., without any prior chromatography step and/or without a prior ultrafiltration step, which, does not lead to substantial loss of viral titres as compared to when the harvest is first concentrated in a membrane filtration step or by means of a prior chromatography step.

The term “feed composition” as used herein refers to the solution that is introduced to the chromatography column or the ultrafiltration membrane. The feed composition is directly related to how the harvest is processed before the chromatography step for example by clarification, or stabilization, or clarification and stabilization. Thus, it has been surprisingly found that directly performing a chromatography step after the clarification, stabilization or clarification and stabilization of the harvest does not lead to substantial loss of viral titer as compared to when the same feed composition is first concentrated in a membrane filtration step such as a TFF step.

Thus, according to an embodiment of the invention, high viral titers can be obtained when a large-scale flaviviral vaccine production and manufacture comprises the following steps sequentially: a chromatography step directly on the clarified and stabilized harvest, and followed by ultrafiltration. In this context, the flavivirus can be a dengue virus. In this context, the vaccine can be a live, attenuated viral vaccine. Preferably, the harvesting step, the clarification of the harvest, the stabilization of the harvest, the purification of the clarified and stabilized harvest with a chromatography step followed by ultrafiltration are all performed on the same day.

Thus, according to an embodiment of the invention, high viral titers can be obtained when a large-scale flaviviral vaccine production and manufacture comprises the following steps sequentially: a chromatography step directly on the clarified and stabilized harvest, and followed by tangential flow filtration. In this context, the flavivirus can be a dengue virus. In this context, the vaccine can be a live, attenuated viral vaccine. Preferably, the harvesting step, the clarification of the harvest, the stabilization of the harvest, the purification of the clarified and stabilized harvest with a chromatography step followed by tangential flow filtration are all performed on the same day.

Thus, according to an aspect of the invention, high viral titers can be obtained when a large-scale flaviviral vaccine production and manufacture comprises the following steps sequentially: an ion exchange chromatography step directly on the clarified and stabilized harvest, and followed by ultrafiltration. In this context, the flavivirus can be a dengue virus. In this context, the vaccine can be a live, attenuated viral vaccine. Preferably, the harvesting step, the clarification of the harvest, the stabilization of the harvest, the purification of the clarified and stabilized harvest with ion exchange chromatography followed by ultrafiltration are all performed on the same day.

Thus, according to an aspect of the invention, high viral titers can be obtained when a large-scale flaviviral vaccine production and manufacture comprises the following steps sequentially: an ion exchange chromatography step directly on the clarified and stabilized harvest, and followed by tangential flow filtration. In this context, the flavivirus can be a dengue virus. In this context, the vaccine can be a live, attenuated viral vaccine. Preferably, the harvesting step, the clarification of the harvest, the stabilization of the harvest, the purification of the clarified and stabilized harvest with ion exchange chromatography followed by tangential flow filtration are all performed on the same day.

Thus, according to an embodiment of the invention, high viral titers can be obtained when a large-scale flaviviral vaccine production and manufacture comprises the following steps sequentially: an anion exchange chromatography step directly on the clarified and stabilized harvest, and followed by ultrafiltration. In this context, the flavivirus can be a dengue virus. In this context, the vaccine can be a live, attenuated viral vaccine. Preferably, the harvesting step, the clarification of the harvest, the stabilization of the harvest, the purification of the clarified and stabilized harvest with anion exchange chromatography followed by ultrafiltration are all performed on the same day.

Thus, according to an embodiment of the invention, high viral titers can be obtained when a large-scale flaviviral vaccine production and manufacture comprises the following steps sequentially: an anion exchange chromatography step directly on the clarified and stabilized harvest, and followed by tangential flow filtration. In this context, the flavivirus can be a dengue virus. In this context, the vaccine can be a live, attenuated viral vaccine. Preferably, the harvesting step, the clarification of the harvest, the stabilization of the harvest, the purification of the clarified and stabilized harvest with anion exchange chromatography followed by tangential flow filtration are all performed on the same day.

Thus, according to an embodiment of the invention, high viral titers can be obtained when a large-scale flaviviral vaccine production and manufacture comprises the following steps sequentially: a cation exchange chromatography step directly on the clarified and stabilized harvest, and followed by ultrafiltration. In this context, the flavivirus can be a dengue virus. In this context, the vaccine can be a live, attenuated viral vaccine. Preferably, the harvesting step, the clarification of the harvest, the stabilization of the harvest, the purification of the clarified and stabilized harvest with cation exchange chromatography followed by ultrafiltration are all performed on the same day.

Thus, according to an aspect of the invention, high viral titers can be obtained when a large-scale flaviviral vaccine production and manufacture comprises the following steps sequentially: a cation exchange chromatography step directly on the clarified and stabilized harvest, and followed by tangential flow filtration. In this context, the flavivirus can be a dengue virus. In this context, the vaccine can be a live, attenuated viral vaccine. Preferably, the harvesting step, the clarification of the harvest, the stabilization of the harvest, the purification of the clarified and stabilized harvest with cation exchange chromatography followed by tangential flow filtration are all performed on the same day.

Thus, according to an embodiment of the invention, high viral titers can be obtained when a large-scale flaviviral vaccine production and manufacture comprises the following steps sequentially: (i) providing cells in growth media, (ii) infecting the cells of step (i) with media comprising a flavivirus at a low MOI, (iii) harvesting to obtain a harvest, (iv) processing the harvest of step (iii) comprising clarification and stabilization of the harvest (v) purifying the processed harvest of step (iv) by the sequential steps of (v-a) ion exchange chromatography directly on the processed harvest, and (v-b) tangential flow filtration on the purified harvest obtained in step (v-a). In this context, the cells can be Vero cells. In this context, the flavivirus can be a dengue virus. In this context, the vaccine can be a live, attenuated viral vaccine. Preferably, steps (iii), (iv) and (v) are all performed on the same day.

Thus, according to an embodiment of the invention, high viral titers can be obtained when a large-scale flaviviral vaccine production and manufacture comprises the following steps sequentially: (i) providing cells in growth media, (ii) infecting the cells of step (i) with media comprising a flavivirus at a low MOI, (iii) harvesting to obtain a harvest, (iv) processing the harvest of step (iii) comprising clarification and stabilization of the harvest (v) purifying the processed harvest of step (iv) by the sequential steps of (v-a) anion exchange chromatography directly on the processed harvest and (v-b) tangential flow filtration on the purified harvest obtained in step (v-a). In this context, the cells can be Vero cells. In this context, the flavivirus can be a dengue virus. In this context, the vaccine can be a live, attenuated viral vaccine. Preferably, steps (iii), (iv) and (v) are all performed on the same day.

Thus, according to an embodiment of the invention, high viral titers can be obtained when a large-scale flaviviral vaccine production and manufacture comprises the following steps sequentially: (i) providing cells in growth media, (ii) infecting the cells of step (i) with media comprising a flavivirus at a low MOI, (iii) harvesting to obtain a harvest, (iv) processing the harvest of step (iii) comprising clarification and stabilization of the harvest (v) purifying the processed harvest of step (iv) by the sequential steps of (v-a) cation exchange chromatography directly on the processed harvest and (v-b) tangential flow filtration on the purified harvest obtained in step (v-a). In this context, the cells can be Vero cells. In this context, the flavivirus can be a dengue virus. In this context, the vaccine can be a live, attenuated viral vaccine. Preferably, steps (iii), (iv) and (v) are all performed on the same day.

Thus, according to an embodiment of the invention, high viral titers can be obtained when a large-scale flaviviral vaccine production and manufacture comprises the following steps sequentially: (i) providing cells in growth media, (ii) static infection of the cells of step (i) in monolayer with media comprising a virus at a low MOI, (iii) harvesting to obtain a harvest, (iv) processing the harvest of step (iii) comprising clarification and stabilization of the harvest (v) purifying the processed harvest of step (iv) by the sequential steps of (v-a) ion exchange chromatography directly on the processed harvest and (v-b) tangential flow filtration on the purified harvest obtained in step (v-a). In this context, the cells can be Vero cells. In this context, the flavivirus can be a dengue virus. In this context, the vaccine can be a live, attenuated viral vaccine. In this context, harvesting comprises a first harvesting step and at least three further harvesting steps. In this context, 12-30 hours prior to the first harvesting step, a media change is carried out. Preferably, steps (iii), (iv) and (v) are all performed on the same day.

Thus, according to an embodiment of the invention, high viral titers can be obtained when a large-scale flaviviral vaccine production and manufacture comprises the following steps sequentially: (i) providing cells in growth media, (ii) static infection of the cells of step (i) in monolayer with media comprising a virus at a low MOI, (iii) harvesting to obtain a harvest, (iv) processing the harvest of step (iii) comprising clarification and stabilization of the harvest (v) purifying the processed harvest of step (iv) by the sequential steps of (v-a) anion exchange chromatography directly on the processed harvest and (v-b) tangential flow filtration on the purified harvest obtained in step (v-a). In this context, the cells can be Vero cells. In this context, the flavivirus can be a dengue virus. In this context, the vaccine can be a live, attenuated viral vaccine. In this context, harvesting comprises a first harvesting step and at least three further harvesting steps. In this context, 12-30 hours prior to the first harvesting step, a media change is carried out. Preferably, steps (iii), (iv) and (v) are all performed on the same day.

Thus, according to an embodiment of the invention, high viral titers can be obtained when a large-scale flaviviral vaccine production and manufacture comprises the following steps sequentially: (i) providing cells in growth media, (ii) static infection of the cells of step (i) in monolayer with media comprising a virus at a low MOI, (iii) harvesting to obtain a harvest, (iv) processing the harvest of step (iii) comprising clarification and stabilization of the harvest (v) purifying the processed harvest of step (iv) by the sequential steps of (v-a) cation exchange chromatography directly on the processed harvest and (v-b) tangential flow filtration on the purified harvest obtained in step (v-a). In this context, the cells can be Vero cells. In this context, the flavivirus can be a dengue virus. In this context, the vaccine can be a live, attenuated viral vaccine. In this context, harvesting comprises a first harvesting step and at least three further harvesting steps. In this context, 12-30 hours prior to the first harvesting step, a media change is carried out Preferably, steps (iii), (iv) and (v) are all performed on the same day.

At the end of one or more chromatography steps, the composition obtained is termed “purified harvest”, which refers to a processed harvest that has been subjected to at least one chromatography steps.

The term “clarified and purified harvest” refers to a crude harvest that has been subjected to a clarification step and one or more chromatography steps in that order. The term “clarified, stabilized and purified harvest” refers to a crude harvest that has been subjected to a clarification step, a stabilization step and one or more chromatography steps in that order. The term stabilized, clarified and purified harvest refers to a crude harvest that has been subjected to a clarification step, one or more chromatography steps and a stabilization step in that order.

In one embodiment, by using the method of the invention as described herein above, a low MOI can be used for large-scale flaviviral vaccine production and manufacture providing higher average viral titres as compared to the same method comprising the same process steps but using a high MOI. Thus, by using the method of the invention, a low MOI can be used for large-scale attenuated flaviviral vaccine production and manufacture providing higher average viral titers as compared to the same method comprising the same process steps but using a high MOI. In this context, high MOI refers to MOI greater than 0.008. In this context, the flavivirus can be a dengue virus. “Average viral titers” as used herein refers to average viral titers of at least two harvesting steps.

Accordingly, in certain embodiments, a method for large-scale flaviviral vaccine production and manufacture is provided comprising the following sequential steps:
(i) providing cells in growth media,
(ii) infecting the cells of step (i) with infection media comprising flavivirus,
(iii) harvesting to obtain a harvest,
(iv) processing the harvest of step (iii) to obtain a processed harvest,
(v) purifying the processed harvest of step (iv), wherein step (v) comprises at least one chromatography step,
wherein the pH throughout steps (iii) to (v) is maintained at a range from 7.6 to 8.1, and
wherein the difference between
(a) the maximum pH occurring at any time point throughout steps (iii) to (v), and
(b) the minimum pH occurring at any time point throughout steps (iii) to (v),
is no greater than 0.4 units.

In certain embodiments, the method results in higher viral yield as compared to the same method with the same process steps but allowing a difference between (a) and (b) to be greater than 0.4 units. In certain embodiments, the higher viral yield pertains to the viral yield that is higher by at least 1 mg/cm2, such as 1.5 mg/cm2.Accordingly, in certain embodiments, a method for large-scale Flaviviral vaccine production and manufacture is provided comprising the following sequential steps:
(i) providing cells in growth media,
(ii) infecting the cells of step (i) with infection media comprising flavivirus,
(iii) harvesting to obtain a harvest,
(iv) processing the harvest of step (iii) to obtain a processed harvest,
(v) purifying the processed harvest of step (iv), wherein step (v) comprises at least one chromatography step,
wherein the pH throughout steps (iii) to (v) is maintained at a range from 7.6 to 8.1, and
wherein the difference between
(a) the maximum pH occurring at any time point throughout steps (iii) to (v), and
(b) the minimum pH occurring at any time point throughout steps (iii) to (v),
is no greater than 0.3 units.

In certain embodiments, the method results in higher viral yield as compared to the same method with the same process steps but allowing a difference between (a) and (b) to be greater than 0.3 units. In certain embodiments, the higher viral yield pertains to the viral yield that is higher by at least 1 mg/cm2, such as 1.5 mg/cm2.

Accordingly, in certain embodiments, a method for large-scale Flaviviral vaccine production and manufacture is provided comprising the following sequential steps:

(i) providing cells in growth media,

(ii) infecting the cells of step (i) with infection media comprising flavivirus at a low MOI,

(iii) harvesting to obtain a harvest,

(iv) processing the harvest of step (iii) to obtain a processed harvest,

(v) purifying the processed harvest of step (iv), wherein step (v) comprises at least one chromatography step,
wherein the pH throughout steps (iii) to (v) is maintained at a range from 7.6 to 8.1, and
wherein the difference between
(a) the maximum pH occurring at any time point throughout steps (iii) to (v), and
(b) the minimum pH occurring at any time point throughout steps (iii) to (v),
is no greater than 0.4 units.

In certain embodiments, “low MOI” pertains to less than 0.008. In certain embodiments, the method results in higher viral yield as compared to the same method with the same process steps but allowing a difference between (a) and (b) to be greater than 0.4 units. In certain embodiments, the higher viral yield pertains to the viral yield that is higher by at least 1 mg/cm2, such as 1.5 mg/cm2.

Accordingly, in certain embodiments, a method for large-scale Flaviviral vaccine production and manufacture is provided comprising the following sequential steps:
(i) providing cells in growth media,
(ii) infecting the cells of step (i) with infection media comprising flavivirus at a low MOI,
(iii) harvesting to obtain a harvest,
(iv) processing the harvest of step (iii) to obtain a processed harvest,
(v) purifying the processed harvest of step (iv), wherein step (v) comprises at least one chromatography step,
wherein the pH throughout steps (iii) to (v) is maintained at a range from 7.6 to 8.1, and
wherein the difference between
(a) the maximum pH occurring at any time point throughout steps (iii) to (v), and
(b) the minimum pH occurring at any time point throughout steps (iii) to (v),
is no greater than 0.3 units.

In certain embodiments, “low MOI” pertains to less than 0.008.

In certain embodiments, the method results in higher viral yield as compared to the same method with the same process steps but allowing a difference between (a) and (b) to be greater than 0.3 units. In certain embodiments, the higher viral yield pertains to the viral yield that is higher by at least 1 mg/cm2, such as 1.5 mg/cm2.

The calculation of virus yield in mg/cm2 can be conducted as follows when one type of flask is used:

$$Total number of flasks \times area of each flask in cm 2 = total area \qquad ( i )$$

$$Total virus yield in milligrams / Total area in cm 2 obtained from step ( i ) , \\ above = virus yield / cm 2 \qquad ( ii )$$

The calculation of virus yield in mg/cm2 can be conducted as follows when more than one type of flask is used:

$$( number of flask type 1 ) \times area of each type 2 flask in cm 2 ) + ( number of flask type 2 ) \times ( area of each type 2 flask in cm 2 ) etc . = total area \qquad ( i )$$

$$Total virus yield in milligrams / Total area in cm 2 obtained from step ( i ) , \\ above = virus yield / cm 2 \qquad ( ii )$$

### Purifying the Processed Harvest: Ultrafiltration

In the context of the invention, the step of purifying the harvest can comprise a step of ultrafiltration to obtain a drug substance.

Thus, the feed composition, i.e., the input of the ultrafiltration step is either a clarified and purified harvest, a clarified, stabilized, and purified harvest, a stabilized, clarified and purified harvest, a stabilized and purified harvest, or only a purified harvest.

The objective of the ultrafiltration step implementation in the method of the invention is to minimize harvest volume without affecting virus potency/recovery. According to an embodiment of the invention, the ultrafiltration step can be a direct flow filtration (DFF) or tangential flow filtration step (TFF).

According to an embodiment of the invention, the method for large-scale viral vaccine production and manufacture comprises the sequential steps of (i) providing cells in growth media, (ii) infecting the cells of step (i) with media comprising a flavivirus at a low MOI, (iii) harvesting to obtain a harvest and (iv) processing the harvest of step (iii) to obtain a processed harvest and (v) purifying the processed harvest of step (iv) comprising at least one chromatography step and an ultrafiltration step. Preferably, steps (iii), and (iv) and (v) are all performed on the same day.

According to an embodiment of the invention, the method for large-scale viral vaccine production and manufacture comprises the sequential steps of (i) providing cells in growth media, (ii) infecting the cells of step (i) with media comprising a flavivirus at a low MOI, (iii) harvesting to obtain a harvest and (iv) processing the harvest of step (iii) to obtain a processed harvest and (v) purifying the processed harvest of step (iv) comprising at least one chromatography step and a direct flow filtration step. Preferably, steps (iii), (iv) and (v) are all performed on the same day.

According to an embodiment of the invention, the method for large-scale viral vaccine production and manufacture comprises the sequential steps of (i) providing cells in growth media, (ii) infecting the cells of step (i) with media comprising a flavivirus at a low MOI, (iii) harvesting to obtain a harvest and (iv) processing the harvest of step (iii) to obtain a processed harvest and (v) purifying the processed harvest of step (iv) comprising at least one chromatography step and a tangential flow filtration step. In this context, the cells can be Vero cells. In this context, the flavivirus can be a dengue virus. In this context, the vaccine can be a live, attenuated viral vaccine. Preferably, steps (iii), (iv) and (v) are all performed on the same day.

Direct flow filtration (DFF) is applied when all the fluid to be filtered is driven, due to a supply pressure, in a direction perpendicular to a filtering surface. Contaminants are captured within the filtration media or build up on the surface, causing the differential pressure across the filter to rise as it blocks over the duration of the filtration process. The filtrate exits the filter on the downstream side. Once a certain differential pressure has been reached, after which the fluid flow rate decreases and/or the filter reaches its terminal differential pressure, filtration is stopped, and the filter is either discarded or may sometimes be regenerated for re-use. On the other hand, Tangential Flow Filtration (TFF) is a process where the feed stream flows parallel to the membrane face. Applied pressure causes one portion of the flow stream to pass through the membrane (filtrate) while the remainder (retentate) is recirculated back to the feed reservoir. Thus, in TFF the majority of the feed travels tangentially across the surface of the filter and not into the filter.

After at least one processing step, the composition obtained can be subjected to one or more tangential flow filtration (TFF) steps, one TFF step and one DFF step, or one TFF step. This step serves to concentrate the sample to ensure there is sufficient potency and to remove small molecular weight impurities.

Suitable filter materials for TFF can be cross-linked cellulose and polyethersulfone. Preferably, a cross-linked cellulose-based polymer is used which is available from Sartorius under the name Hydrosart. The cut-off size of the filter material used in tangential flow filtration determines whether a certain compound is present in the filtrate or the retentate. The cut-off size may be smaller than 1/3 to 1/5 of the size of the virus to be purified. For an embodiment of the method of the invention, the cut-off size can be between 50 and 300 kDa. Within the range of 50 and 300 kDa, the cut-off size can be 100 kDa. Accordingly, for the purpose of the present invention, a cross-linked cellulose-based polymer filter with a cut-off size of 100 kDa can be used in the TFF step. Typical TFF membranes used for this step include molecular weight cut-off (MWCO) sizes of 100 kDa (Hydrosart) and 300 kDa polyethersulfone (PES).

The TFF process can be characterized by the flow rate and the transmembrane pressure. The flow rate is proportional to the transmembrane pressure and inverse proportional to the resistance of the membrane and the filter cake. The flow rate is given as the feed flow rate per unit area of membrane which is liter per meter squared per hour (LMH). In the TFF step of the present invention the flow rate can be between 100 and 500 LMH. Within the range of 100 and 500 LMH, the flow rate can be between 120 and 400 LMH, between 150 and 300 LMH or 300 LMH. The transmembrane pressure (TMP) is the pressure difference between two sides of the membrane. The TMP may be 0.2 to 0.5 bar, 0.25 to 0.45 bar, 0.30 to 0.45 bar, 0.35 to 0.45 bar, or 0.35 to 0.40 bar, or 0.30 to 0.50 bar.

Two basic filter configurations are generally used for TFF: cartridge filters and cassette filters. In cartridge filters (often called hollow fiber filters), the membrane forms a set of parallel hollow fibers. The feed stream passes through the lumen of the fibers and the permeate is collected from outside the fibers. In cassette filters, several flat sheets of membrane are held apart from each other and from the cassette housing by support screens. The feed stream passes into the space between two sheets and permeate is collected from the opposite side of the sheets. In embodiments of the TFF step in the method of the present invention, both a cartridge or cassette filter can be used. In other embodiments of the TFF step in the method of the present invention only a cartridge filter can be used. Alternatively. in the TFF step in the method of the present invention only a cassette filter can be used.

In the TFF step the solution containing the viruses for flaviviral vaccine manufacture is in the retentate. The retentate obtained is also the drug substance. After the TFF step, the feed volume can e.g. be concentrated two- to ten-folds, three- to eight-fold, four- to six-fold or five-fold. At the end of the TFF step, a drug substance is obtained. In one embodiment, the feed can be a clarified and purified harvest. In one embodiment, the feed can be a clarified, stabilized, and purified harvest. In one embodiment, the feed can be a stabilized, clarified and purified harvest. In one embodiment, the feed can be a stabilized and purified harvest. In one embodiment, the feed can be a purified harvest.

In certain embodiments, the pH throughout the steps mentioned above is maintained in a range from 7.6 to 8.1.

Accordingly, in certain embodiments, a method for large-scale Flaviviral vaccine production and manufacture is provided comprising the following sequential steps:
(i) providing cells in growth media,
(ii) infecting the cells of step (i) with infection media comprising flavivirus,
(iii) harvesting to obtain a harvest,
(iv) processing the harvest of step (iii) to obtain a processed harvest,
(v) purifying the processed harvest of step (iv), wherein step (v) comprises the following sequential steps:
(v-a) ion exchange chromatography to obtain a purified harvest preferably, anion exchange chromatography,
(v-b) ultrafiltration to obtain a drug substance preferably, tangential flow filtration,
wherein the pH throughout steps (iii) to (v) is maintained at a range from 7.6 to 8.1, and
wherein the difference between
(a) the maximum pH occurring at any time point throughout steps (iii) to (v), and
(b) the minimum pH occurring at any time point throughout steps (iii) to (v),
is no greater than 0.4 units.

In certain embodiments, the method results in higher viral yield as compared to the same method with the same process steps but allowing a difference between (a) and (b) to be greater than 0.4 units. In certain embodiments, the higher viral yield pertains to the viral yield that is higher by at least 1 mg/cm2, such as 1.5 mg/cm2.

Accordingly, in certain embodiments, a method for large-scale Flaviviral vaccine production and manufacture is provided comprising the following sequential steps:
(i) providing cells in growth media,
(ii) infecting the cells of step (i) with infection media comprising flavivirus,
(iii) harvesting to obtain a harvest,
(iv) processing the harvest of step (iii) to obtain a processed harvest,
(v) purifying the processed harvest of step (iv), wherein step (v) comprises the following sequential steps:
(v-a) ion exchange chromatography to obtain a purified harvest preferably, anion exchange chromatography,
(v-b) ultrafiltration to obtain a drug substance preferably, tangential flow filtration,
wherein the pH throughout steps (iii) to (v) is maintained at a range from 7.6 to 8.1, and
wherein the difference between
(a) the maximum pH occurring at any time point throughout steps (iii) to (v), and
(b) the minimum pH occurring at any time point throughout steps (iii) to (v),
is no greater than 0.3 units.

In certain embodiments, the method results in higher viral yield as compared to the same method with the same process steps but allowing a difference between (a) and (b) to be greater than 0.3 units. In certain embodiments, the higher viral yield pertains to the viral yield that is higher by at least 1 mg/cm2, such as 1.5 mg/cm2.

Accordingly, in certain embodiments, a method for large-scale Flaviviral vaccine production and manufacture is provided comprising the following sequential steps:
(i) providing cells in growth media,
(ii) infecting the cells of step (i) with infection media comprising flavivirus at a low MOI,
(iii) harvesting to obtain a harvest,
(iv) processing the harvest of step (iii) to obtain a processed harvest,
(v) purifying the processed harvest of step (iv), wherein step (v) comprises the following sequential steps:
(v-a) ion exchange chromatography to obtain a purified harvest preferably, anion exchange chromatography,
(v-b) ultrafiltration to obtain a drug substance preferably, tangential flow filtration,
wherein the pH throughout steps (iii) to (v) is maintained at a range from 7.6 to 8.1, and
wherein the difference between
(a) the maximum pH occurring at any time point throughout steps (iii) to (v), and
(b) the minimum pH occurring at any time point throughout steps (iii) to (v), is no greater than 0.4 units.

In certain embodiments, “low MOI” pertains to less than 0.008. In certain embodiments, the method results in higher viral yield as compared to the same method with the same process steps but allowing a difference between (a) and (b) to be greater than 0.4 units. In certain embodiments, the higher viral yield pertains to the viral yield that is higher by at least 1 mg/cm2, such as 1.5 mg/cm2.

Accordingly, in certain embodiments, a method for large-scale Flaviviral vaccine production and manufacture is provided comprising the following sequential steps:
(i) providing cells in growth media,
(ii) infecting the cells of step (i) with infection media comprising flavivirus at a low MOI,
(iii) harvesting to obtain a harvest,
(iv) processing the harvest of step (iii) to obtain a processed harvest,
(v) purifying the processed harvest of step (iv), wherein step (v) comprises the following sequential steps:
(v-a) ion exchange chromatography to obtain a purified harvest preferably, anion exchange chromatography,
(v-b) ultrafiltration to obtain a drug substance preferably, tangential flow filtration,
wherein the pH throughout steps (iii) to (v) is maintained at a range from 7.6 to 8.1, and
wherein the difference between
(a) the maximum pH occurring at any time point throughout steps (iii) to (v), and
(b) the minimum pH occurring at any time point throughout steps (iii) to (v), is no greater than 0.3 units.
   In certain embodiments, “low MOI” pertains to less than 0.008.
   In certain embodiments, the method results in higher viral yield as compared to the same method with the same process steps but allowing a difference between (a) and (b) to be greater than 0.3 units. In certain embodiments, the higher viral yield pertains to the viral yield that is higher by at least 1 mg/cm2, such as 1.5 mg/cm2.

Accordingly, an embodiment of the method for large-scale flaviviral vaccine production and manufacture of flaviviral vaccines includes a step of processing the harvest and a step of purifying the processed harvest comprising at least one chromatography step followed by an ultrafiltration step. The purification step comprises an ultrafiltration step after the chromatography step. At the end of the ultrafiltration step, a drug substance is obtained. If a divalent, trivalent, or tetravalent final drug product is intended, the drug substance at this step still comprises each individual monovalent drug substance. In the context of the invention, it is to be understood that each of the individual method steps and/or components of ingredients described herein above are contemplated alone or in combination with each other. Thus, each possibility described herein above alone and in combination with each other represents a separate embodiment of the invention.

### Processing the Drug Substance to Obtain a Bulk Drug Substance

According to an embodiment of the invention, the method for large-scale flaviviral vaccine production and manufacture comprises the sequential steps of (i) providing cells in growth media, (ii) infecting the cells of step (i) with media comprising a flavivirus at a low MOI, (iii) harvesting to obtain a harvest and (iv) processing the harvest of step (iii) to obtain a processed harvest and (v) purifying the processed harvest of step (iv) comprising at least one chromatography step to obtain a purified harvest (v) an ultrafiltration step to obtain a drug substance (vi) processing the drug substance obtained in step (v) comprising at least one flushing step of the ultrafiltration membrane with a flushing buffer to obtain a composition comprising the buffer flush and drug substance which is the bulk drug substance. Preferably, the ultrafiltration is tangential flow filtration. Preferably, the chromatography step is anion exchange chromatography. In this context, the cells can be Vero cells. In a preferred embodiment, the flaviviral vaccine is a tetravalent dengue vaccine including all four live, attenuated dengue serotypes including dengue serotype 1 such as dengue 2/1 chimera or TDV-1 represented by SEQ ID NO: 1 and/or SEQ ID NO:2, dengue serotype 2 or TDV-2 represented by SEQ ID NO: 3 and/or SEQ ID NO:4, dengue serotype 3 such as dengue serotype 2/3 or TDV-3 represented by SEQ ID NO: 5 and/or SEQ ID NO:6, dengue serotype 4 such as dengue serotype 2/4 or TDV-4 represented by SEQ ID NO: 7 and/or SEQ ID NO:8. The methods described in this section are performed for each of the four serotypes separately.

The TFF step for the purpose of the invention may or may not be followed by diafiltration. Diafiltration is a fractionation process that washes smaller molecules through a membrane and leaves larger molecules in the retentate without ultimately changing concentration. It can be used to remove salts or exchange buffers. In preferred embodiments the method of the invention includes a step of flushing the filter membrane with buffer one, two, three, four or five times to obtain a composition comprising the buffer flush and drug substance, which is the bulk drug substance. Preferably, the harvesting step, the clarification of the harvest, the stabilization of the harvest, the purification of the clarified and stabilized harvest with a chromatography step and tangential flow filtration followed by the step of flushing are all performed on the same day.

The TFF step for the purpose of the invention may or may not be followed by diafiltration. Diafiltration is a fractionation process that washes smaller molecules through a membrane and leaves larger molecules in the retentate without ultimately changing concentration. It can be used to remove salts or exchange buffers. In an exemplary embodiment the method of the invention includes a step of flushing the filter membrane with buffer one, two, three, four or five times to obtain a buffer flush Preferably, the harvesting step, the clarification of the harvest, the stabilization of the harvest, the purification of the clarified and stabilized harvest with a chromatography step and tangential flow filtration followed by the step of flushing are all performed on the same day.

After the TFF step, the composition comprising the drug substance and the buffer flush is the bulk drug substance. Thus, according to an embodiment of the invention, a bulk drug substance is a composition comprising the drug substance (TFF retentate) and at least two buffer flushes of the TFF membrane. Thus, according to an embodiment of the invention, a bulk drug substance is a composition comprising the drug substance (TFF retentate) and at least three buffer flushes of the TFF membrane. Thus, according to an embodiment of the invention, a bulk drug substance is a composition comprising the drug substance (TFF retentate) and at least four buffer flushes of the TFF membrane.

After the TFF step, the drug substance and the buffer flush can be mixed to obtain a bulk drug substance. Thus, according to an exemplary embodiment of the invention, a bulk drug substance is a composition comprising the drug substance (TFF retentate) and at least two buffer flushes of the TFF membrane. Thus, according to an embodiment of the invention, a bulk drug substance is a composition comprising the drug substance (TFF retentate) and at least three buffer flushes of the TFF membrane. Thus, according to an embodiment of the invention, a bulk drug substance is a composition comprising the drug substance (TFF retentate) and at least four buffer flushes of the TFF membrane.

The flushing buffer comprises the same buffered excipient composition/stabilization buffer added at the step of processing the harvest, more specifically at the step of stabilization of the harvest except that the excipients in the flushing buffer are at a lower concentration as compared to the buffered excipient composition/stabilization buffer.

The flushing buffer comprises one or more excipients e.g., including a non-ionic surfactant, a non-reducing sugar and an albumin that are all described in the section of stabilization of the harvest. The flushing buffer used for flushing can comprise F 127, trehalose dihydrate and human serum albumin. The buffer used for flushing preferably comprises 0.5 to 5% of a non-ionic surfactant, 20 to 40% of a non-reducing sugar and 0.05 to 0.3% of albumin. The buffer used for flushing preferably comprises 0.5 to 5% of F 127, 20 to 40% of trehalose dihydrate and 0.05 to 0.3% of human serum albumin.

The flushing buffer can comprise the same excipients as that of the stabilization buffer added at step (ii) of the method including sugar, non-ionic surfactant, and protein. Thus, in a preferred embodiment, the flushing buffer comprises the same excipients as that of the stabilization buffer added at step (ii) of the method including sugar, non-ionic surfactant, and protein except that the excipients in the flushing buffer are at a lower concentration as compared to the excipients in the stabilization buffer.

The flushing buffer can comprise the same excipients as that of the stabilized or purified harvest including sugar, non-ionic surfactant, and protein. Thus, in a preferred embodiment, the flushing buffer comprises the same excipients as that of the stabilized or purified harvest including sugar, non-ionic surfactant, and protein except that the excipients in the flushing buffer are at a higher concentration as compared to the excipients in the stabilized or purified harvest.

The flushing buffer can comprise the same excipients as that of the drug substance including sugar, non-ionic surfactant, and protein. In a preferred embodiment, the flushing buffer comprises the same excipients as that of the drug substance including sugar, non-ionic surfactant, and protein except that the sugar in the flushing buffer is at a higher concentration as compared to the sugar in the drug substance.

Thus, an embodiment of the method for large-scale production and manufacture of flaviviral vaccine includes further step (vi) of processing the drug substance comprising at least one flushing step of the ultrafiltration membrane with a flushing buffer to obtain a composition comprising the buffer flush and the drug substance, which is the bulk drug substance, wherein the flushing buffer comprises a sugar, a surfactant, and a protein. Preferably, steps (iii), (iv), (v), and (vi) are all performed on the same day.

In one exemplary embodiment, the method for large-scale production and manufacture of flaviviral vaccine includes further step (vi) of processing the drug substance comprising at least one flushing step of the ultrafiltration membrane with a flushing buffer to obtain a buffer flush, wherein the flushing buffer comprises a sugar, a surfactant, and a protein. Preferably, steps (iii), (iv), (v), and (vi) are all performed on the same day.

According to an embodiment of the method of the invention, the drug substance and the bulk drug substance comprise the same excipients as that of the stabilization buffer added at step (ii) of the method including sugar, non-ionic surfactant, and protein. In a preferred embodiment, the concentration of the excipients including sugar, non-ionic surfactant, and protein in the drug substance and bulk drug substance is the same.

In one embodiment, the concentration of the excipients in the bulk drug substance from the buffered excipient composition/stabilization buffer added at step (ii) is two to five times higher as compared to the concentration of these excipients in the stabilized harvest or the purified harvest. In a preferred embodiment, the concentration of these excipients in the bulk drug substance is five times higher as compared to the concentration of these excipients in the stabilized harvest or the purified harvest.

In one embodiment, the concentration of the excipients in the bulk drug substance from the buffered excipient composition/stabilization buffer added at step (ii) including a sugar, a protein and/or a non-ionic surfactant is two to five times higher as compared to the concentration of these excipients in the stabilized harvest or the purified harvest. In a preferred embodiment, the concentration of these excipients in the bulk drug substance is five times higher as compared to the concentration of these excipients in the stabilized harvest or the purified harvest.

In certain embodiments, the pH throughout the steps mentioned above is maintained in a range from 7.6 to 8.1.

Accordingly, in certain embodiments, a method for large-scale Flaviviral vaccine production and manufacture is provided comprising the following sequential steps:
(i) providing cells in growth media,
(ii) infecting the cells of step (i) with infection media comprising flavivirus,
(iii) harvesting to obtain a harvest,
(iv) processing the harvest of step (iii) to obtain a processed harvest,
(v) purifying the processed harvest of step (iv), wherein step (v) comprises the following sequential steps:
(v-a) ion exchange chromatography to obtain a purified harvest preferably, anion exchange chromatography,
(v-b) ultrafiltration to obtain a drug substance preferably, tangential flow filtration,
(vi) processing the drug substance obtained from step (v-b) to obtain a bulk drug substance,
wherein the pH throughout steps (iii) to (vi) is maintained at a range from 7.6 to 8.1, and
wherein the difference between
(a) the maximum pH occurring at any time point throughout steps (iii) to (vi), and
(b) the maximum pH occurring at any time point throughout steps (iii) to (vi),
is no greater than 0.4 units.
   In certain embodiments, the method results in higher viral yield as compared to the same method with the same process steps but allowing a difference between (a) and (b) to be greater than 0.4 units. In certain embodiments, the higher viral yield pertains to the viral yield that is higher by at least 1 mg/cm², such as 1.5 mg/cm².

Accordingly, in certain embodiments, a method for large-scale Flaviviral vaccine production and manufacture is provided comprising the following sequential steps:
(i) providing cells in growth media,
(ii) infecting the cells of step (i) with infection media comprising flavivirus,
(iii) harvesting to obtain a harvest,
(iv) processing the harvest of step (iii) to obtain a processed harvest,
(v) purifying the processed harvest of step (iv), wherein step (v) comprises the following sequential steps:
(v-a) ion exchange chromatography to obtain a purified harvest preferably, anion exchange chromatography,
(v-b) ultrafiltration to obtain a drug substance preferably, tangential flow filtration,
(vi) processing the drug substance obtained from step (v-b) to obtain a bulk drug substance,
wherein the pH throughout steps (iii) to (vi) is maintained at a range from 7.6 to 8.1, and
wherein the difference between
(a) the maximum pH occurring at any time point throughout steps (iii) to (vi), and
(b) the maximum pH occurring at any time point throughout steps (iii) to (vi),
is no greater than 0.3 units.
   In certain embodiments, the method results in higher viral yield as compared to the same method with the same process steps but allowing a difference between (a) and (b) to be greater than 0.3 units. In certain embodiments, the higher viral yield pertains to the viral yield that is higher by at least 1 mg/cm², such as 1.5 mg/cm².

Accordingly, in certain embodiments, a method for large-scale Flaviviral vaccine production and manufacture is provided comprising the following sequential steps:
(i) providing cells in growth media,
(ii) infecting the cells of step (i) with infection media comprising flavivirus at a low MOI,
(iii) harvesting to obtain a harvest,
(iv) processing the harvest of step (iii) to obtain a processed harvest,
(v) purifying the processed harvest of step (iv), wherein step (vi) comprises the following sequential steps:
(v-a) ion exchange chromatography to obtain a purified harvest preferably, anion exchange chromatography,
(v-b) ultrafiltration to obtain a drug substance preferably, tangential flow filtration,
(vi) processing the drug substance obtained from step (v-b) to obtain a bulk drug substance,
wherein the pH throughout steps (iii) to (v) is maintained at a range from 7.6 to 8.1, and
wherein the difference between
(a) the maximum pH occurring at any time point throughout steps (iii) to (vi), and
(b) the maximum pH occurring at any time point throughout steps (iii) to (vi),
is no greater than 0.4 units.
   In certain embodiments, “low MOI” pertains to less than 0.008. In certain embodiments, the method results in higher viral yield as compared to the same method with the same process steps but allowing a difference between (a) and (b) to be greater than 0.4 units. In certain embodiments, the higher viral yield pertains to the viral yield that is higher by at least 1 mg/cm², such as 1.5 mg/cm².

Accordingly, in certain embodiments, a method for large-scale Flaviviral vaccine production and manufacture is provided comprising the following sequential steps:
(i) providing cells in growth media,

(ii) infecting the cells of step (i) with infection media comprising flavivirus at a low MOI,

(iii) harvesting to obtain a harvest,

(iv) processing the harvest of step (iii) to obtain a processed harvest,

(v) purifying the processed harvest of step (iv), wherein step (v) comprises the following sequential steps:
(v-a) ion exchange chromatography to obtain a purified harvest preferably, anion exchange chromatography,
(v-b) ultrafiltration to obtain a drug substance preferably, tangential flow filtration,
(vi) processing the drug substance obtained from step (v-b) to obtain a bulk drug substance,
wherein the pH throughout steps (iii) to (v) is maintained at a range from 7.6 to 8.1, and
wherein the difference between
(a) the maximum pH occurring at any time point throughout steps (iii) to (vi), and
(b) the maximum pH occurring at any time point throughout steps (iii) to (vi),
is no greater than 0.3 units.
   In certain embodiments, “low MOI” pertains to less than 0.008.
   In certain embodiments, the method results in higher viral yield as compared to the same method with the same process steps but allowing a difference between (a) and (b) to be greater than 0.3 units. In certain embodiments, the higher viral yield pertains to the viral yield that is higher by at least 1 mg/cm², such as 1.5 mg/cm².

Accordingly, an embodiment of the method for large-scale production and manufacture of flaviviral vaccines includes a step of processing the drug substance comprising mixing the drug substance with a buffer flush to obtain a bulk drug substance. The flushing step results in a composition that comprises the buffer flush and the drug substance, which is the bulk drug substance. If a divalent, trivalent, or tetravalent final drug product is intended, the bulk drug substance at this step still comprises each individual monovalent bulk drug substance. In the context of the invention, it is to be understood that each of the individual method steps and/or components or ingredients described herein above are contemplated alone or in combination with each other. Thus, each possibility described herein above alone and in combination with each other represents a separate embodiment of the invention.

### Processing the Bulk Drug Substance

According to an embodiment of the invention, the method for large-scale flaviviral vaccine production and manufacture comprises the sequential steps of (i) providing cells in growth media, (ii) infecting the cells of step (i) with media comprising a flavivirus at a low MOI, (iii) harvesting to obtain a harvest and (iv) processing the harvest of step (iii) to obtain a processed harvest and (v) purifying the processed harvest of step (iv) comprising at least one chromatography step to obtain a purified harvest (v) an ultrafiltration step to obtain a drug substance (vi) processing the drug substance obtained in step (v) preferably comprising at least one flushing step of the ultrafiltration membrane with a flushing buffer to obtain a bulk drug substance and (vii) processing the bulk drug substance. Preferably, the ultrafiltration is tangential flow filtration. Preferably, the chromatography step is anion exchange chromatography. In this context, the cells can be Vero cells. In a preferred embodiment, the flaviviral vaccine is a tetravalent dengue vaccine including all four live, attenuated dengue serotypes including dengue serotype 1 such as dengue 2/1 chimera or TDV-1 represented by SEQ ID NO: 1 and/or SEQ ID NO:2, dengue serotype 2 or TDV-2 represented by SEQ ID NO: 3 and/or SEQ ID NO:4, dengue serotype 3 such as dengue serotype 2/3 or TDV-3 represented by SEQ ID NO: 5 and/or SEQ ID NO:6, dengue serotype 4 such as dengue serotype 2/4 or TDV-4 represented by SEQ ID NO: 7 and/or SEQ ID NO:8. The methods described in this section are performed for each of the four serotypes separately.

Processing the bulk drug substance may comprise a depth filtration step. This filtration step can be carried out in the same manner as the step of clarification of the harvest.

Accordingly, depth filtration uses a porous filtration medium to separate particles and solids from a liquid. The filter media which may be used in depth filtration can be cellulose acetate, polypropylene, cellulose acetate protected by glassfiber fleece and polyethersulfone. The filter used in depth filtration can have a pore size of 0.1 μm to 1 μm, 0.2 μm to 0.8 μm, or 0.2 μm to 0.45 μm.

The filters used in the clarification of the harvest can be heterogenous double layer of polyethersulfone with pore sizes of 0.2 μm and 0.45 μm. Thus, for the purpose of clarification of the harvest, several heterogenous double layer filters can be used such as Sartoclean (Cellulose Acetate) 0.8+0.65 μm, Sartopure (Polyporpylene) 0.65 μm, Sartoclean (Glassfiber Fleeces) 0.8+0.65 μm, Sartopore 2 (Polyethersulfone) 0.8+0.45 μm, Sartopore 2 (Polyethersulfone) 0.45+0.2 μm, or Sartopore 2 XLG (Polyethersulfone) 0.8+0.2 μm. Each possibility represents a separate embodiment of the invention.

According to the method of the invention, preferably, the bulk drug substance after this step comprises the same excipients as present in the bulk drug substance before the processing step.

Freezing is another way of processing the bulk drug substance and can be used to store and freeze the bulk drug substance preferably below −30° C.

Thawing is another way of processing the bulk drug substance and refers to adapting the frozen bulk drug substance to room temperature.

Pooling is another way of processing the bulk drug substance and comprises pooling two or more bulk drug substances.

In one embodiment of the method of the invention, a first harvest obtained from the first harvesting step is clarified and stabilized on the same day. The clarified and stabilized harvest is also purified on the same day with at least one chromatography step, preferably an anion exchange chromatography. The purified harvest is also subjected to an ultrafiltration step on the same day, preferably a tangential flow filtration to obtain a first drug substance. The first drug substance is also mixed on the same day with a flushing buffer to obtain a composition comprising the buffer flush and the first drug substance, which is the first bulk drug substance. In one embodiment, 20-30 hours after the first harvesting step, a second harvesting step is conducted, and the above steps are repeated to obtain a second bulk drug substance. In one embodiment, up to ten harvesting steps are conducted, and the above steps are repeated to obtain up to ten bulk drug substances, wherein the interval between each harvesting step is 20-30 hours.

In a preferred embodiment, six harvesting steps are conducted and the above steps are repeated to obtain six bulk drug substances on six different days, wherein the interval between each harvesting step is 20-30 hours.

In a preferred embodiment, the above steps are conducted in the same way for each of the four live, attenuated dengue serotypes including including dengue serotype 1 such as dengue 2/1 chimera or TDV-1 represented by SEQ ID NO: 1 and/or SEQ ID NO:2, dengue serotype 2 or TDV-2 represented by SEQ ID NO: 3 and/or SEQ ID NO:4, dengue serotype 3 such as dengue serotype 2/3 or TDV-3 represented by SEQ ID NO: 5 and/or SEQ ID NO: 6, dengue serotype 4 such as dengue serotype 2/4 or TDV-4 represented by SEQ ID NO: 7 and/or SEQ ID NO:8.

Accordingly, in a preferred embodiment, for dengue serotype 1, six bulk drug substances are obtained from six harvesting steps, each bulk drug substance obtained at the end of each day of harvest, wherein the interval between each harvesting step is 20-30 hours. Accordingly, in a preferred embodiment, for dengue serotype 2 six bulk drug substances are obtained from six harvesting steps, each bulk drug substance obtained at the end of each day of harvest, wherein the interval between each harvesting step is 20-30 hours. Accordingly, in a preferred embodiment, for dengue serotype 3 six bulk drug substances are obtained from six harvesting steps, each bulk drug substance obtained at the end of each day of harvest, wherein the interval between each harvesting step is 20-30 hours. Accordingly, in a preferred embodiment, for dengue serotype 4 six bulk drug substances are obtained from six harvesting steps, each bulk drug substance obtained at the end of each day of harvest, wherein the interval between each harvesting step is 20-30 hours.

In one embodiment, each bulk drug substance can be frozen on the same day or subjected to a step of depth filtration on the same day and then frozen on the same day.

In a preferred embodiment, each bulk drug substance of dengue serotype 1 is immediately frozen on the same day. In a preferred embodiment, each of the frozen bulk drug substances of dengue serotype 1 are thawed, pooled, and then subjected to a step of depth filtration. In a preferred embodiment, six bulk drug substances of dengue serotype 1 are thawed, pooled, and then subjected to a step of depth filtration.

In a preferred embodiment, each bulk drug substance of dengue serotype 2 is immediately frozen on the same day. In a preferred embodiment, each of the frozen bulk drug substances of dengue serotype 2 are thawed, pooled, and then subjected to a step of depth filtration. In a preferred embodiment, six bulk drug substances of dengue serotype 2 are thawed, pooled, and then subjected to a step of depth filtration.

In a preferred embodiment, each bulk drug substance of dengue serotype 3 is immediately subjected to a step of depth filtration and then frozen on the same day. In a preferred embodiment, six bulk drug substances of dengue serotype 3 are subjected to a step of depth filtration individually and then frozen on the same day individually.

In a preferred embodiment, each bulk drug substance of dengue serotype 4 is immediately subjected to a step of depth filtration and then frozen on the same day. In a preferred embodiment, six bulk drug substances of dengue serotype 4 are subjected to a step of depth filtration individually and then frozen on the same day individually.

In a preferred embodiment, the method of the invention includes the following steps for dengue serotype 1 such as dengue 2/1 chimera or TDV-1 represented by SEQ ID NO: 1 and/or SEQ ID NO:2, (i) providing cells in growth media as explained in the earlier section, (ii) infection of cells with media comprising dengue serotype 1 at a low MOI as described in the earlier section, (iii) a first harvest obtained from the first harvesting step (iv) processing the first harvest comprising clarification and stabilization of the harvest on the same day (v) clarified and stabilized harvest is purified on the same day comprising (v-a) at least one chromatography step, preferably an anion exchange chromatography and (v-b) purified harvest is also subjected to an ultrafiltration step on the same day, preferably a tangential flow filtration to obtain a first drug substance (vi) the first drug substance is also processed on the same say, preferably mixed on the same day with a flushing buffer to obtain a composition comprising the buffer flush and the first drug substance, which is the first bulk drug substance (vii) processing the first bulk drug substance comprising (vii-a) freezing the first bulk drug substance to obtain a frozen first bulk drug substance. In a preferred embodiment, six harvesting steps are conducted and the above steps are repeated to obtain six bulk drug substances on six different days, wherein the interval between each harvesting step is 20-30 hours. In one embodiment, the method includes step (vii-b) of thawing the six bulk drug substances, and (vii-c) pooling the six bulk drug substances, and (vii-d) subject the pooled six bulk drug substances to a depth filtration step, and (vii-e) freezing the filtered bulk drug substances.

In a preferred embodiment, the method of the invention includes the following steps for dengue serotype 2 or TDV-2 represented by SEQ ID NO: 3 and/or SEQ ID NO:4, (i) providing cells in growth media as explained in the earlier section, (ii) infection of cells with media comprising dengue serotype 2 at a low MOI as described in the earlier section, (iii) a first harvest obtained from the first harvesting step (iv) processing the first harvest comprising clarification and stabilization of the harvest on the same day (v) clarified and stabilized harvest is purified on the same day comprising (v-a) at least one chromatography step, preferably an anion exchange chromatography and (v-b) purified harvest is also subjected to an ultrafiltration step on the same day, preferably a tangential flow filtration to obtain a first drug substance (vi) the first drug substance is also processed on the same say, preferably mixed on the same day with a flushing buffer to obtain a composition comprising the buffer flush and the first drug substance, which is the first bulk drug substance (vii) processing the first bulk drug substance comprising (vii-a) freezing the first bulk drug substance to obtain a frozen first bulk drug substance. In a preferred embodiment, six harvesting steps are conducted and the above steps are repeated to obtain six bulk drug substances on six different days, wherein the interval between each harvesting step is 20-30 hours. In one embodiment, the method includes step (vii-b) of thawing the six bulk drug substances, and (vii-c) pooling the six bulk drug substances, and (vii-d) subject the pooled six bulk drug substances to a depth filtration step, and (vii-e) freezing the filtered bulk drug substances.

In a preferred embodiment, the method of the invention includes the following steps for dengue serotype 3 or TDV-3 represented by SEQ ID NO: 5 and/or SEQ ID NO:6, (i) providing cells in growth media as explained in the earlier section, (ii) infection of cells with media comprising dengue serotype 3 at a low MOI as described in the earlier section, (iii) a first harvest obtained from the first harvesting step (iv) processing the first harvest comprising clarification and stabilization of the harvest on the same day (v) clarified and stabilized harvest is purified on the same day comprising (v-a) at least one chromatography step, preferably an anion exchange chromatography and (v-b) purified harvest is also subjected to an ultrafiltration step on the same day, preferably a tangential flow filtration to obtain a first drug substance (vi) the first drug substance is also processed on the same day, preferably mixed on the same day with a flushing buffer to obtain a composition comprising the buffer flush and the first drug substance, which is the first bulk drug substance (vii) processing the first bulk drug substance comprising (vii-a) subjecting the first bulk drug substance to a depth filtration step, and (vii-b) freezing the filtered first bulk drug substance. In a preferred embodiment, six harvesting steps are conducted and the above steps are repeated to obtain six bulk drug substances on six different days, wherein the interval between each harvesting step is 20-30 hours.

In a preferred embodiment, the method of the invention includes the following steps for dengue serotype 4 or TDV-4 represented by SEQ ID NO: 7 and/or SEQ ID NO:8, (i) providing cells in growth media as explained in the earlier section, (ii) infection of cells with media comprising dengue serotype 4 at a low MOI as described in the earlier section, (iii) a first harvest obtained from the first harvesting step (iv) processing the first harvest comprising clarification and stabilization of the harvest on the same day (v) clarified and stabilized harvest is purified on the same day comprising (v-a) at least one chromatography step, preferably an anion exchange chromatography and (v-b) purified harvest is also subjected to an ultrafiltration step on the same day, preferably a tangential flow filtration to obtain a first drug substance (vi) the first drug substance is also processed on the same day preferably mixed on the same day with a flushing buffer to obtain a composition comprising the buffer flush and the first drug substance, which is the first bulk drug substance (vii) processing the first bulk drug substance comprising (vii-a) subjecting the first bulk drug substance to a depth filtration step, and (vii-b) freezing the filtered first bulk drug substance. In a preferred embodiment, six harvesting steps are conducted and the above steps are repeated to obtain six bulk drug substances on six different days, wherein the interval between each harvesting step is 20-30 hours.

Accordingly, an embodiment of the method for large-scale production and manufacture of flaviviral vaccines includes a step of processing the bulk drug substance comprising a depth filtration step. If a divalent, trivalent, or tetravalent final drug product is intended, the bulk drug substance at this step still comprises each individual monovalent bulk drug substance. In the context of the invention, it is to be understood that each of the individual method steps and/or components or ingredients described herein above are contemplated alone or in combination with each other. Thus, each possibility described herein above alone and in combination with each other represents a separate embodiment of the invention.

### Freeze and Storage of Bulk Drug Substance

The method of the invention also provides means and ways to freeze and store the bulk drug substance.

The bulk drug substance can be frozen and stored at any time. One such time point is after the filtration of the BDS. After the filtration step, the bulk drug substance can be frozen and stored. The objective of the freezing step is to ensure the viral potency in order for the material to be used for drug product formulation. In this step, any of the commercially available bags can be used to freeze and store the bulk drug substance.

In one embodiment, the bulk drug substance is frozen and stored after the filtration of the BDS as discussed in the previous section, e.g., for TDV-3 and TDV-4. In one embodiment, the bulk drug substance can be immediately frozen and stored after obtaining the bulk drug substance from each harvesting step, thereafter, the bulk drug substances from each harvesting step are thawed, pooled and filtered as discussed above and then frozen and stored e.g., for TDV-1 and TDV-2.

The bags are used to freeze the bulk drug substance and store them until they are thawed for use in the formulation of the drug product. The bags can be selected based on certain parameters such as size, manufacturer, material of construction, and storage temperature range, thaw conditions, ability to maintain sterile conditions, ease of transportation and use in previous processes and clinical studies.

The supported temperature range for the bags can e.g. be within the range −84° C. to 45° C. The freezing options supported by the bags used in the method of the invention can be ultra-low freezer, blast freezer, plate freezing and thawing. “Ultra-low freezer” or “ULTs” as used herein refers to a freezer in operating within the −50° C. to −80° C. range, ULTs are used to store a variety of analytes and products, from biospecimen samples to enzymes and drugs. “Blast freezer” as used herein refers to a specialized freezer that allows a product to be frozen rapidly over a short period of time. The temperature of a blast freezer can vary from −10° C. to −120° C. “Plate freezing” as used herein is a fast-freezing method, once the product is in direct contact with two cold metallic surfaces.

The thawing options supported by the bags used in certain embodiments of the method of the invention can be water bath, plasma thawing bath, shaker incubator, plate freeze-thawing. The term “water bath” refers to a bath containing a heating medium having at least 50% of water by volume. The maximum heating temperature of the water bath can be controlled by selecting the composition of the heating medium. “Plasma thawing” as used herein refers to a method of thawing using an equipment designed for rapid and uniform thawing of fresh frozen plasma (FFP) bags at 37° C. Typically, the device is made of a stainless-steel chamber and equipped with digital temperature controller and a water circulating pump. The considerations for handling and transportation of the bags used in the present invention can be rigidity of the bottles and hard casting that minimizes damage, EVOH gas barrier with EVA film layer, ULDPE film. The material compatibility and sterility characteristics of the bags include free of animal components, gamma irradiated, and USP standards. “EVOH” barrier refers to an environmentally friendly substance that is used as a barrier plastic. Unlike PVDC, EVOH does not contain chlorine, dioxins, metals, or other elements that may cause endocrinological disorders. “GULPED” as used herein refers to ultra-low-density polyethylene.

Accordingly, suitable bags are commercially available bags that can be used to freeze the bulk drug substance including Nalgene PETG/PFA bottles (50 mL-2 L), Sartorius-Stedim Celsius-Pak/FFT/FFTp, 2D bags (30 mL-16 L) or Pall Allegro 2D Biocontainers (125 mL-50 L).

Once a bag is selected, the freezing and thawing conditions must be optimized to minimize the loss of potency of the virus. These conditions include, freezing set point and rate, thawing set point and rate, and optionally cooling set point.

Accordingly, the large-scale production and manufacture of flaviviral vaccines comprises processing the bulk drug substance such as freezing and storing the bulk drug substance. If a divalent, trivalent, or tetravalent final drug product is intended, the bulk drug substance at this step still comprises each individual monovalent bulk drug substance. In the context of the invention, it is to be understood that each of the individual method steps and/or components of ingredients described herein above are contemplated alone or in combination with each other. Thus, each possibility described herein above alone and in combination with each other represents a separate embodiment of the invention.

### Formulation of a Bulk Drug Product

After the bulk drug substance has been frozen, the next step is (ix) formulation of the bulk drug product.

This can e.g. include two major sub-steps:
(ix-a) bulk drug substance thawing
(ix-b) Mixing the thawed bulk drug substance of step (ix-a) and a formulation buffer to obtain a bulk drug product.

If a divalent, trivalent, or tetravalent final drug product is intended, it is at this stage that two or more monovalent bulk drug substances are mixed to obtain a final bulk drug product comprising a divalent, trivalent, or tetravalent drug product. Thus, in one embodiment, after step (ix-a) and before step (ix-b), the two, three, or four thawed bulk drug substances are mixed. In a preferred embodiment, the flaviviral vaccine is a tetravalent dengue vaccine including all four live, attenuated dengue serotypes including dengue serotype 1 such as dengue 2/1 chimera or TDV-1 represented by SEQ ID NO: 1 and/or SEQ ID NO: 2, dengue serotype 2 or TDV-2 represented by SEQ ID NO: 3 and/or SEQ ID NO:4, dengue serotype 3 such as dengue serotype 2/3 or TDV-3 represented by SEQ ID NO: 5 and/or SEQ ID NO:6, dengue serotype 4 such as dengue serotype 2/4 or TDV-4 represented by SEQ ID NO: 7 and/or SEQ ID NO:8.

The formulation buffer comprises two buffers, the first excipient buffer (FEB) and a second excipient buffer (SEB). Qualitatively, the components of each of FEB and SEB may be the same or different. However, quantitatively, the components of each of FEB and SEB are different.

As used herein, the term “excipient” refers to a substance added to a liquid pharmaceutical composition in addition to the biological active agent. This can include substances used for the purpose of enhancing stabilization of the active agent, salts, carbohydrates (such as e.g., sugars), surfactants, proteins, bulking agents, fillers, or agents that in combination with the active agent can confer a therapeutic enhancement of the composition. In particular, excipients may refer to salts, carbohydrates, non-ionic surfactants and albumins. Excipients may also refer to buffers such as e.g., phosphate buffer.

The FEB and SEB buffers can e.g. comprise four salts, a sugar, a non-ionic surfactant, and a protein. The FEB and SEB buffer can also comprise water for injection (WFI) as needed. The salts used in the buffer can be anhydrous or hydrates. The salts can be either one, two, three, or four sodium salts, or one, two, three, or four potassium salts, or one, two, three, or four magnesium salts. Each possibility can be combined with the other possibility. Thus, each possibility represents a separate embodiment of the invention.

For example, the sodium salt can be sodium fluoride, or sodium chloride, or sodium bromide, or sodium iodide, or sodium sulphate, or disodium salts such as disodium phosphate, or sodium dihydrogen phosphate or disodium hydrogen phosphate or disodium hydrogen phosphate, dihydrate, or sodium bicarbonate or sodium carbonate. For example, the potassium salt can be potassium fluoride, or potassium chloride, or potassium iodide, or potassium sulphate, or potassium dihydrogen phosphate or potassium dihydrogen sulphate or potassium bicarbonate or potassium carbonate. For example, the magnesium salt can be magnesium fluoride, or magnesium chloride, or magnesium iodide, or magnesium sulphate, or magnesium dihydrogen phosphate or magnesium bicarbonate or magnesium carbonate.

Each of the above salts can e.g., be used within the range of 0.9 mM to 130 mM in the SEB buffer.

For example, the SEB buffer can comprise sodium chloride in the range 100 to 130 mM, 110 to 130 mM, 120 to 130 mM and other three salts within the range of 0.9 to 6 mM. For example, the SEB buffer can comprise potassium chloride in the range 100 to 130 mM, 110 to 130 mM, 120 to 130 mM and other three salts within the range of 0.9 to 6 mM. For example, the SEB and SEB buffer can comprise magnesium chloride in the range 100 to 130 mM, 110 to 130 mM, 120 to 130 mM and other three salts within the range of 0.9 to 6 mM.

For example, the SEB buffer can comprise sodium fluoride in the range 100 to 130 mM, 110 to 130 mM, 120 to 130 mM and other three salts within the range of 0.9 to 6 mM. For example, the SEB buffer can comprise potassium fluoride in the range 100 to 130 mM, 110 to 130 mM, 120 to 130 mM and other three salts within the range of 0.9 to 6 mM.

For example, the SEB buffer can comprise sodium carbonate in the range 100 to 130 mM, 110 to 130 mM, 120 to 130 mM and other three salts within the range of 0.9 to 6 mM. For example, the SEB buffer can comprise potassium carbonate in the range 100 to 130 mM, 110 to 130 mM, 120 to 130 mM and other three salts within the range of 0.9 to 6 mM.

For example, the SEB buffer can comprise sodium chloride in the range 100 to 130 mM, 110 to 130 mM, 120 to 130 mM, and potassium chloride in the range 1 mM to 2 mM, 1 mM to 1.5 mM and two other salts within the range of 0.9 to 6 mM. For example, the SEB buffer can comprise sodium chloride in the range 100 to 130 mM, 110 to 130 mM, 120 to 130 mM, and magnesium chloride in the range 1 mM to 2 mM, 1 mM to 1.5 mM and two other salts within the range of 0.9 to 6 mM. For example, the SEB buffer can comprise potassium chloride in the range 100 to 130 mM, 110 to 130 mM, 120 to 130 mM, and magnesium chloride in the range 1 mM to 2 mM, 1 mM to 1.5 mM and two other salts within the range of 0.9 to 6 mM.

For example, the SEB buffer can comprise sodium fluoride in the range 100 to 130 mM, 110 to 130 mM, 120 to 130 mM, and potassium chloride in the range 1 mM to 2 mM, 1 mM to 1.5 mM and two other salts within the range of 0.9 to 6 mM. For example, the SEB buffer can comprise sodium fluoride in the range 100 to 130 mM, 110 to 130 mM, 120 to 130 mM, and potassium carbonate in the range 1 mM to 2 mM, 1 mM to 1.5 mM and two other salts within the range of 0.9 to 6 mM. For example, the SEB buffer can comprise sodium fluoride in the range 100 to 130 mM, 110 to 130 mM, 120 to 130 mM, and magnesium chloride in the range 1 mM to 2 mM, 1 mM to 1.5 mM and two other salts within the range of 0.9 to 6 mM. For example, the SEB buffer can comprise sodium iodide in the range 100 to 130 mM, 110 to 130 mM, 120 to 130 mM, and potassium carbonate in the range 1 mM to 2 mM, 1 mM to 1.5 mM and two other salts within the range of 0.9 to 6 mM.

For example, the SEB buffer can comprise sodium chloride in the range 100 to 130 mM, 110 to 130 mM, 120 to 130 mM, and potassium chloride in the range 1 mM to 2 mM, 1 mM to 1.5 mM, potassium dihydrogen phosphate within the range 0.9 to 1.2 mM, 0.9 to 1.0 mM and one other salt within the range of 4 to 6 mM, 5 to 6 mM. For example, the SEB buffer can comprise sodium chloride in the range 100 to 130 mM, 110 to 130 mM, 120 to 130 mM, and potassium phosphate in the range 1 mM to 2 mM, 1 mM to 1.5 mM, potassium dihydrogen sulphate within the range 0.9 to 1.2 mM, 0.9 to 1.0 mM and one other salt within the range of 4 to 6 mM, 5 to 6 mM. For example, the SEB buffer can comprise sodium chloride in the range 100 to 130 mM, 110 to 130 mM, 120 to 130 mM, and potassium phosphate in the range 1 mM to 2 mM, 1 mM to 1.5 mM, sodium dihydrogen sulphate within the range 0.9 to 1.2 mM, 0.9 to 1.0 mM and one other salt within the range of 4 to 6 mM, 5 to 6 mM.

For example, the SEB buffer can comprise sodium chloride in the range 100 to 130 mM, 110 to 130 mM, 120 to 130 mM, and potassium chloride in the range 1 mM to 2 mM, 1 mM to 1.5 mM, potassium dihydrogen phosphate within the range 0.9 to 1.2 mM, 0.9 to 1.0 mM and disodium hydrogen phosphate dihydrate within the range of 4 to 6 mM, 5 to 6 mM. For example, the SEB buffer can comprise potassium chloride in the range 100 to 130 mM, 110 to 130 mM, 120 to 130 mM, and magnesium chloride in the range 1 mM to 2 mM, 1 mM to 1.5 mM, sodium dihydrogen phosphate within the range 0.9 to 1.2 mM, 0.9 to 1.0 mM and disodium hydrogen phosphate dihydrate within the range of 4 to 6 mM, 5 to 6 mM. For example, the SEB buffer can comprise potassium chloride in the range 100 to 130 mM, 110 to 130 mM, 120 to 130 mM, and magnesium chloride in the range 1 mM to 2 mM, 1 mM to 1.5 mM, sodium dihydrogen phosphate within the range 0.9 to 1.2 mM, 0.9 to 1.0 mM and magnesium carbonate within the range of 4 to 6 mM, 5 to 6 mM.

Each of the above salts can be used within the range of 3 mM to 50 mM in the FEB buffer.

For example, the FEB buffer can comprise sodium chloride in the range 20 to 50 mM, 30 to 50 mM, 40 to 50 mM and other three salts within the range of 3 to 25 mM. For example, the FEB buffer can comprise potassium chloride in the range 20 to 50 mM, 30 to 50 mM, 40 to 50 mM and other three salts within the range of 3 to 25 mM. For example, the FEB buffer can comprise magnesium chloride in the range 20 to 50 mM, 30 to 50 mM, 40 to 50 mM and other three salts within the range of 3 to 25 mM.

For example, the FEB buffer can comprise sodium fluoride in the range 20 to 50 mM, 30 to 50 mM, 40 to 50 mM and other three salts within the range of 3 to 25 mM. For example, the FEB buffer can comprise potassium fluoride in the range 20 to 50 mM, 30 to 50 mM, 40 to 50 mM and other three salts within the range of 3 to 25 mM.

For example, the FEB buffer can comprise sodium carbonate in the range 20 to 50 mM, 30 to 50 mM, 40 to 50 mM and other three salts within the range of 3 to 25 mM. For example, the FEB buffer can comprise potassium carbonate in the range 20 to 50 mM, 30 to 50 mM, 40 to 50 mM and other three salts within the range of 3 to 25 mM.

For example, the FEB buffer can comprise sodium chloride in the range range 20 to 50 mM, 30 to 50 mM, 40 to 50 mM, and potassium chloride in the range 4 to 6 mM, 5 to 6 mM and two other salts within the range of 3 to 25 mM. For example, the FEB buffer can comprise sodium chloride in the range range 20 to 50 mM, 30 to 50 mM, 40 to 50 mM, and magnesium chloride in the range 4 to 6 mM, 5 to 6 mM and two other salts within the range of 3 to 25 mM. For example, the FEB buffer can comprise potassium chloride in the range range 20 to 50 mM, 30 to 50 mM, 40 to 50 mM, and magnesium chloride in the range 4 to 6 mM, 5 to 6 mM and two other salts within the range of 3 to 25 mM.

For example, the FEB buffer can comprise sodium fluoride in the range 20 to 50 mM, 30 to 50 mM, 40 to 50 mM, and potassium chloride in the range 4 to 6 mM, 5 to 6 mM and two other salts within the range of 3 to 25 mM. For example, the FEB buffer can comprise sodium fluoride in the range 20 to 50 mM, 30 to 50 mM, 40 to 50 mM, and potassium carbonate in the range 4 to 6 mM, 5 to 6 mM and two other salts within the range of 3 to 25 mM. For example, the FEB buffer can comprise sodium fluoride in the range 20 to 50 mM, 30 to 50 mM, 40 to 50 mM, and magnesium chloride in the range 4 to 6 mM, 5 to 6 mM and two other salts within the range of 3 to 25 mM. For example, the FEB buffer can comprise sodium iodide in the range 20 to 50 mM, 30 to 50 mM, 40 to 50 mM, and potassium carbonate in the range 4 to 6 mM, 5 to 6 mM and two other salts within the range of 3 to 25 mM.

For example, the FEB buffer can comprise sodium chloride in the range range 20 to 50 mM, 30 to 50 mM, 40 to 50 mM, and potassium chloride in the range 4 to 6 mM, 5 to 6 mM, potassium dihydrogen phosphate within the range 3 to 4 mM, 3.2 to 4 mM and one other salt within the range of 18 to 22 mM. For example, the FEB buffer can comprise sodium chloride in the range range 20 to 50 mM, 30 to 50 mM, 40 to 50 mM, and potassium phosphate in the range 4 to 6 mM, 5 to 6 mM, potassium dihydrogen sulphate within the range 3 to 4 mM, 3.2 to 4 mM and one other salt within the range of 18 to 22 mM. For example, the FEB buffer can comprise sodium chloride in the range range 20 to 50 mM, 30 to 50 mM, 40 to 50 mM, and potassium phosphate in the range 4 to 6 mM, 5 to 6 mM, sodium dihydrogen sulphate within the range 3 to 4 mM, 3.2 to 4 mM and one other salt within the range of 18 to 22 mM.

For example, the FEB buffer can comprise sodium chloride in the range 20 to 50 mM, 30 to 50 mM, 40 to 50 mM, and potassium chloride in the range 4 to 6 mM, 5 to 6 mM, potassium dihydrogen phosphate within the range 3 to 4 mM, 3.2 to 4 mM and disodium hydrogen phosphate dihydrate within the range of 18 to 22 mM, 19 to 22 mM, 20 to 22 mM. For example, the FEB buffer can comprise potassium chloride in the range 20 to 50 mM, 30 to 50 mM, 40 to 50 mM, and magnesium chloride in the range 4 to 6 mM, 5 to 6 mM, sodium dihydrogen phosphate within the range 3 to 4 mM, 3.2 to 4 mM and disodium hydrogen phosphate dihydrate within the range of 18 to 22 mM, 19 to 22 mM, 20 to 22 mM. For example, the FEB buffer can comprise potassium chloride in the range 20 to 50 mM, 30 to 50 mM, 40 to 50 mM, and magnesium chloride in the range 4 to 6 mM, 5 to 6 mM, sodium dihydrogen phosphate within the range 3 to 4 mM, 3.2 to 4 mM and magnesium carbonate within the range of 18 to 22 mM, 19 to 22 mM, 20 to 22 mM.

The sugar in the FEB and SEB buffer can be monosaccharides, (e.g. glucose, galactose, ribose, mannose, rhamnose, talose, xylose, or allose arabinose.), disaccharides (e.g. trehalose, sucrose, maltose, isomaltose, cellibiose, gentiobiose, laminaribose, xylobiose, mannobiose, lactose, or fructose), trisaccharides (e.g. acarbose, raffinose, melizitose, panose, or cellotriose) and sugar polymers (e.g. dextran, xanthan, pullulan, cyclodextrins, amylose, amylopectin, starch, cello-oligosaccharides, cellulose, maltooligosaccharides, glycogen, chitosan, or chitin). The sugar in the FEB and SEB buffer can be sugar alcohols such as mannitol, sorbitol, arabitol, erythritol, maltitol, xylitol, glycitol, glycol, polyglycitol, polyethylene glycol, polypropylene glycol, and glycerol. The sugar in the FEB and SEB buffer can be a non-reducing sugar. The sugar in the FEB and SEB buffer can be sucrose, trehalose or its hydrates such as trehalose dihydrate.

Each of the above sugars can be used within the range of 120 to 160 g/L in the SEB buffer and within the range of 180 to 220 g/L in the FEB buffer. For example, sucrose can be within the range of 120 to 160 g/L, 130 to 160 g/L, 140 to 150 g/L in the SEB buffer and 180 to 220 g/L, 190 to 210 g/L, 200 to 210 g/L in the FEB buffer. For example, trehalose can be within the range of 120 to 160 g/L, 130 to 160 g/L, 140 to 150 g/L in the SEB buffer and 180 to 220 g/L, 190 to 210 g/L, 200 to 210 g/L in the FEB buffer. For example, trehalose dihydrate can be within the range of 120 to 160 g/L, 130 to 160 g/L, 140 to 150 g/L in the SEB buffer and 180 to 220 g/L, 190 to 210 g/L, 200 to 210 g/L in the FEB buffer.

The non-ionic surfactant in the FEB and SEB buffer can be selected from block copolymers, sorbitan esters, ethoxylated or propoxylated sorbitan esters, alkyl-polyglycosides (APG), alkoxylated mono- or di-alkylamines, fatty acid monoethanolamides (FAMA), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), and combinations thereof.

The non-ionic surfactant in the FEB and SEB buffer can be a high molecular weight non-ionic surfactant. The non-ionic surfactant can be a non-ionic triblock copolymer. The surfactant can be a non-ionic, hydrophilic, polyoxyethylene-polyoxypropylene block copolymer (or EO-PO block copolymer). The EO-PO block copolymers can include blocks of polyethylene oxide (—CH2CH2O-designated EO) and polypropylene oxide (—CH2CHCH30-designated PO). The PO block can be flanked by two EO blocks in a EOx-POy-Eox arrangement. Since the PO component is hydrophilic and the EO component is hydrophobic, the overall hydrophilicity, molecular weight and the surfactant properties of the copolymer can be adjusted by varying x and y in the EOx-POy-Eox block structure. In aqueous solutions, the EO-PO block copolymers will self-assemble into micelles with a PO core and a corona of hydrophilic EO groups.

The non-ionic surfactant in the FEB and SEB buffer can be a poloxamer.

Poloxamers are non-ionic triblock copolymers composed of a central hydrophobic chain of poly(propyleneoxide) flanked by two hydrophilic chains of poly(ethylene oxide). The length of the polymer blocks can be customized, leading to different poloxamers with slightly different properties. The non-ionic surfactant in the FEB and SEB buffer can be Pluronic F127 (poloxamer 407), Pluronic F68 (poloxamer 403), Pluronic P123, Pluronic P85, other polyethylene oxide-polypropylene oxide (EO-PO) block copolymers of greater than 3,000-4,000 MW or combinations thereof.

The concentration of the non-ionic surfactant in the FEB and SEB buffer can be the same. The concentration of the non-ionic surfactant in the FEB and SEB buffer each can be with the range 2.0 to 12 g/L, 4 to 12 g/L, 6 to 12 g/L, 8 to 12 g/L, or 10 to 12 g/L. The concentration of a high molecular weight non-ionic surfactant in the FEB and SEB buffer each can be with the range 2.0 to 12 g/L, 4 to 12 g/L, 6 to 12 g/L, 8 to 12 g/L, or 10 to 12 g/L. The concentration of a poloxamer in the FEB and SEB buffer each can be with the range 2.0 to 12 g/L, 4 to 12 g/L, 6 to 12 g/L, 8 to 12 g/L, or 10 to 12 g/L. The concentration of Pluronic F127 (poloxamer 407) in the FEB and SEB buffer each can be with the range 2.0 to 12 g/L, 4 to 12 g/L, 6 to 12 g/L, 8 to 12 g/L, or 10 to 12 g/L. The concentration of Pluronic F68 (poloxamer 403) in the FEB and SEB buffer each can be with the range 2.0 to 12 g/L, 4 to 12 g/L, 6 to 12 g/L, 8 to 12 g/L, or 10 to 12 g/L. The concentration of Pluronic P123 in the FEB and SEB buffer each can be with the range 2.0 to 12 g/L, 4 to 12 g/L, 6 to 12 g/L, 8 to 12 g/L, or 10 to 12 g/L. The concentration of a Pluronic P85 in the FEB and SEB buffer each can be with the range 2.0 to 12 g/L, 4 to 12 g/L, 6 to 12 g/L, 8 to 12 g/L, or 10 to 12 g/L.

The protein in the FEB and SEB buffer can be any protein which is essentially inert and does not react with the virus. In particular, the protein does not affect the structure or infectivity of the virus. Thus, the protein can be a structural protein or a serum protein. The protein can be selected from an albumin, human serum albumin, collagen, hydrolyzed collagen, gelatin and hydrolyzed gelatin.

The concentration of the protein in the FEB and SEB buffer can be the same.

The concentration of protein in the FEB and SEB buffer each can be within the range 0.5 to 1.5 g/L, 0.7 to 1.2 g/L, 0.8 to 1.0 g/L. The concentration of protein in the FEB and SEB buffer each can be within the range 0.5 to 1.5 g/L, 0.7 to 1.2 g/L, 0.8 to 1.0 g/L. The concentration of albumin in the FEB and SEB buffer each can be within the range 0.5 to 1.5 g/L, 0.7 to 1.2 g/L, 0.8 to 1.0 g/L. The concentration of human serum albumin in the FEB and SEB buffer each can be within the range 0.5 to 1.5 g/L, 0.7 to 1.2 g/L, 0.8 to 1.0 g/L. The concentration of collagen in the FEB and SEB buffer each can be within the range 0.5 to 1.5 g/L, 0.7 to 1.2 g/L, 0.8 to 1.0 g/L. The concentration of gelatin in the FEB and SEB buffer each can be within the range 0.5 to 1.5 g/L, 0.7 to 1.2 g/L, 0.8 to 1.0 g/L.

In the context of the invention, it is to be understood that each of the individual components of the buffer described herein above are contemplated alone or in combination with each other. Once the bulk drug substance is thawed, it is mixed with FEB and SEB to formulate the bulk drug product.

Accordingly, one embodiment of the large-scale production and manufacture of flaviviral vaccines, comprises formulation of a bulk drug product. If a divalent, trivalent, or tetravalent final drug product is intended, the drug product obtained at this stage comprises a divalent, trivalent or tetravalent bulk drug product. In the context of the invention, it is to be understood that each of the individual method steps and/or components of ingredients described herein above are contemplated alone or in combination with each other. Thus, each possibility described herein above alone and in combination with each other represents a separate embodiment of the invention.

### Sterile Filtration

The formulated and mixed bulk drug product material can be filtered through a sterile filtration assembly. The pore size of the filter can be up to a maximum of 0.2 μm. The specific types and characteristics of the filters that can be used have been discussed previously under clarification of the harvest and can also be used in this step.

For the purpose of the invention, sterile filtration of the bulk drug product can be conducted via a peristaltic pump characterized by up to 260 rpm and a maximum inlet pressure of 1.0 to 1.2 bar. The filtered drug product can be collected in a separate vessel that is held at a temperature within the range 2° C. to 8° C. For the purpose of the invention, the sterile filtered bulk drug product can be mixed at approximately 100 to 150 rpm for at least 10 minutes and then further sampled for sterility.

Accordingly, the large-scale production and manufacture of flaviviral vaccines may comprise sterile filtration of the bulk drug product. In the context of the invention, it is to be understood that each of the individual method steps and/or components described herein above are contemplated alone or in combination with each other. Thus, each possibility described herein above alone and in combination with each other represents a separate embodiment of the invention.

### Filling and Loading

The filling process can be conducted using any commercial scale isolator filling line known in the art. Filling machine isolators are designed to enclose the filling, stoppering and capping operations of a Powder or Liquid Filling Machine while providing an aseptic environment for the process. This provides increased product safety by reducing the risk of contamination with no direct contact between operators and product. The isolators also provide good, environmentally friendly decontamination options.

According to the method of the invention, during filling, the temperature of the isolator filling line is maintained at 10° C. to 25° C., more preferably between 15° C. to 25° C.

The method of the invention utilizes vials that can be used to fill the filtered drug product. The vials can be either plastic or glass vials. In a preferred embodiment, the vials are glass vials. When glass vials are used, any of the USP Type I, II or III glass vials can be used. In a preferred embodiment, USP Type I glass vials are used.

“Glass” as used herein refers to any suitable glass. For example, neutral glass is a borosilicate glass containing significant amounts of boric oxide, aluminum oxide, alkali and/or alkaline earth oxides. It has a high hydrolytic resistance and a high thermal shock resistance. Soda-lime-silica glass is a silica glass containing alkali metal oxides, mainly sodium oxide and alkaline earth oxides, mainly calcium oxide. It has only a moderate hydrolytic resistance.

According to their hydrolytic resistance, glass containers are classified as:
Type I glass containers which are of neutral glass, with a high hydrolytic resistance, suitable for most preparations whether or not for parenteral use,
Type II glass containers which are usually of soda-lime-silica glass with high hydrolytic resistance resulting from suitable treatment of the surface. They are suitable for most acidic and neutral, aqueous preparations whether or not for parenteral use,
Type III glass containers which are usually of soda-lime-silica glass with only moderate hydrolytic resistance. They are generally suitable for non-aqueous preparations for parenteral use, for powders for parenteral use (except for freeze-dried preparations) and for preparations not for parenteral use.

In a preferred embodiment, the vials used in the present invention are made of borosilicate glass. In a preferred embodiment, the vials used in the present invention have high hydrolytic resistance and a high thermal shock resistance. Thus, in a preferred embodiment, the vials used are USP Type I glass vials.

The USP Type I glass vials which can be used in the present invention can be either 2R or 4R tubular glass vials with a capacity of 2 mL or 4 mL, respectively.

Thus, according to an embodiment of the invention, a USP Type I 2R vial or a USP Type 1 2R vial can be used to fill the filtered bulk drug product. Prior to filling, a number of steps can be carried including but not limited to one or more of washing of the vials, sterilization of the vials, or de-pyrogenation of the vials. Alternatively, the method can use pre-sterilized glass supplied directly to the filling station as a cost- and space-saving alternative to operating a washing and sterilizing line. Pre-sterilized glass vials are washed and depyrogenated by the supplier, double bagged and then gamma irradiated to sterilize before being shipped to the end user for use.

Once the vials have been processed, they are reading for filling. At this time, the sterile filtered bulk is aseptically connected to the single use filling manifold. Prior to filling, the bulk can be mixed at approximately 100 to 150 rpm, 110 to 120 rpm, 130 to 140 rpm to 150 rpm for a minimum of 20 to 30 minutes, 15 to 20 minutes, or 15 minutes. In a preferred embodiment, mixing is carried out at 140 to 150 rpm, for about 15 minutes.

In a preferred embodiment of the invention, the mixing and filling continue in parallel with each other. Preferably, the mixing continues for 15 minutes until approximately 60 to 80% of the vials are filled, for example, 70 to 80%, 75 to 80% of the vials are filled. In another embodiment, after the first 15 minutes, or the first 20 minutes of mixing and filling, the mixing is switched off. In this embodiment, the mixing is not continued in parallel with the further filling. Thus, in one embodiment, at least 60 to 80% of the vials for example, 70 to 80%, 75 to 80% vials are filled in parallel with the mixing of bulk drug product.

The filling range is one of the important parameters of this step in certain embodiments of the method of the invention. The critical vial fill volume can be set according to the circumstances and the required use. For example, critical vial fill volume can be set at 0.5 to 0.8 mL, 0.6 to 0.7 mL, 0.65 to 0.66 mL, 0.651, 0.652, 0.653, 0.654, or 0.655 mL. A skilled person knows how to set a critical vial fill volume by manually setting the pump seeds for the filling needles in the isolator filling line at the initiation of the fill run and then maintaining the volume by automatic feedback control. In an embodiment of the method of the invention, additional checks are incorporated to ensure that the content remains within the critical fill volume. These checks can be for example weight checks.

Thus, the method of the invention may or may not include weight checks to confirm that the volume is within the critical fill volume. If weight checks are employed, a standard deviation of +5 to 7% may be considered as fulfilling the requirements of the critical fill volume.

Once the filling is completed, one embodiment of the method of the invention involves stoppering of the vial. In one embodiment, the filling machine may partially stopper the vials. In this embodiment, the partial stoppering is accomplished before transferring the vial into the freeze-drying units. The stoppers may or may not undergo a step of sterilization before being transferred to the filler. In one preferred embodiment, the transfer is carried out aseptically. After the step of partial stoppering, a step of “proper stoppering” inspection may or may not be implemented. At the end of filling the minimum product yield is confirmed to be at least 90%, 95% or 97% good vials out of total number of filled vials.

The total time in solution post formulation (post-TIS) is defined as the time that begins with the end of transfer of the last lot per serotype and ends at beginning of lyophilization cycle. The post-TIS may be within the range of 10-30 hours, more preferably 12-29 hours. It is possible that the post-TIS for one serotype is different as compared to the post-TIS for another serotype. In one embodiment, the post-TIS for TDV-4 is the longest, the post-TIS for TDV 2 is the shortest or the same as TDV-1 and TDV-3, and the post-TIS for TDV-1 and TDV-3 is the same. In this embodiment, the the post-TIS for TDV-4 is within the range of 15-30 hours, 16-30 hours, or 16-29 hours, the post-TIS for TDV 2 is within the rage 10-24 hours, or 12-24 hours, and the post-TIS for TDV-1 and TDV-3 is 11-25 hours, or 12-25 hours.

Accordingly, the large-scale production and manufacture of flaviviral vaccines may comprise filling and loading the bulk drug product in vials. In the context of the invention, it is to be understood that each of the individual method steps and/or components described herein above are contemplated alone or in combination with each other. Thus, each possibility described herein above alone and in combination with each other represents a separate embodiment of the invention.

### Lyophilization and Seal

The drug product can be lyophilized, such as in commercial scale freeze-dryers. Lyophilization involves manipulating the temperature and pressure of the solution so that the phase of the solution can move directly from the frozen state to the gaseous state without moving through the liquid phase/state. This is achieved by cooling the solution and lowering the pressure to below the triple point of water (the temperature and pressure at which water can exist in equilibrium in the liquid, solid, and gaseous states-which is 0.01° C.). This allows for the removal of the solvent from the product without subjecting the product to intense heat.

The lyophilization process cycle for freeze-drying liquid is e.g. comprised of five primary phases: equilibration, freezing, primary drying, secondary drying, and stoppering, each controlled by three basic process parameters: temperature, pressure, and time.

Thus, one embodiment of the method of the invention comprises a freeze-drying method for preparing a dry composition, the method comprising the steps of (i) providing an aqueous composition, (ii) freezing the aqueous composition to form a frozen composition, (iii) subjecting the frozen composition to a primary drying step in an apparatus with a shelf temperature, which ranges from above the Tg of the composition to not more than 15° C. above the Tg and at a pressure below 0.3 mbar to form a primary dried product, and (iv) subjecting the primary dried product to a secondary drying step at a temperature above the temperature of the primary drying step to form a secondary dried product.

According to one embodiment of the method of the invention, the primary drying step (iii) is carried out in an apparatus with a shelf temperature of from −33° C. to −10° C., or from −33° C. to −20° C., or from −31° C. to −23° C., or from 29° C. to −25° C., such as about −27° C. In this context, the primary drying step (c) can be carried out in an apparatus with a shelf temperature above the collapse temperature of the composition (Tc). Further, the primary drying step (iii) is carried out for a period of from 100 hours to 10 hours, or from 60 hours to 30 hours, or from 50 hours to 40 hours, such as around 48 hours. Further, the primary drying step (iii) can be carried out at a pressure of less than 0.1 mbar, or less than 0.05 mbar, or less than 0.025 mbar, and optionally above about 0.01 mbar. Further, the secondary drying step (iv) can be carried out at a temperature of at least 20° C., or at least 25° C.

Accordingly, the large-scale production and manufacture of flaviviral vaccines may comprise lyophilization of the drug product. In the context of the invention, it is to be understood that each of the individual method steps and/or components described herein above are contemplated alone or in combination with each other. Thus, each possibility described herein above alone and in combination with each other represents a separate embodiment of the invention.

### Unloading and Capping

Upon completion of secondary drying the vials can be capped. For example: the chamber pressure can be backfilled to −0.125 to −0.075 bar (partial vacuum) for stoppering using dry sterile nitrogen gas (N2). Vials can be stoppered with ≥6.5 N/cm² pressure for 60 seconds and the shelf temperature is then decreased to 3° C. to 7° C. until unloaded into a Grade A environment.

The vials can be sealed, such as with aluminium/plastic, e.g. while in the sterile isolator.

### Visual Inspection

Every vial is visually inspected either manually or through a semi-automated process for potential defects of product, glass, crimping, stoppering or others including but not limited to cake appearance, cap defects, glass defects, and visible particles.

### Freezing Final Drug Product

It is preferred that the drug product is frozen for storage. Each drug product batch can e.g, be transferred to an additional storage location at 2° C. to 8° C. before being frozen, or the product batch can be transferred directly to a dedicated blast freezer, for example with temperature −15° C. to −35° C., for blast freezing which can last e.g., at least 36 hours blast freezer. The batch can e.g. remain in the blast freezer for at least 36 hours.

After blast freezing, drug product batches can be transferred to long-term storage, for example at −15° C. to −25° C.

### EXAMPLES

### Example 1-Low MOI Range

This example shows that high viral titers can be obtained in large scale flaviviral vaccine production and manufacture when cells in monolayer are infected with infection media comprising a flavivirus at a low MOI (less than 0.008, or 0.005 or less) irrespective of the volume of infection media used.

A vial of Vero WCB is thawed in 37° C. water bath. The thawing should not exceed 5 minutes. Post-thawed cells are then transferred into appropriate tissue culture vessels. Vero cells were grown in tissue culture vessels comprising growth media.

The major components of Vero cell growth media-Dulbecco growth media (DGM)—consists of Dulbecco's Modified Eagle's Medium (DMEM), glutamine and fetal bovine serum (FBS). The growth media comprised DMEM+4.0 mM glutamine+3.52 g/L glucose+10% fetal bovine serum (FBS). Vero cells were cultured at 37° C., 5% CO₂, with operating ranges of 36° C. to 39° C. and 4% to 6% respectively.

Vero cells were grown to at least 90% confluency on the surface of the culture vessel prior to TDV infection. When the cells are confluent, the cells are dislodged from the surface by TrypLE Select within 10 to 30 minutes. When the desired cell density had reached, infection and virus production was performed using the infection media. The infection media comprises DMEM+4.0 mM glutamine+3.88 g/L glucose+0.1% (w/v) of F127. The volume of infection media was between 0.014 to 0.114 mL/cm².

Infection media, as described above, comprising monovalent TDV-1, 2, 3 or 4 working virus seed (WVS) at low MOI (in this example 0.005 and 0.001) and high MOI (in this example 0.01 and 0.05) was used for the infection of Vero cells in monolayer in individual tissue culture vessels.

The volume of infection media used was between 0.014 to 0.114 mL/cm² and the method of infection was rocking/shaking of the tissue culture vessels. Thus, leading to the following groups as shown in Table 1 below.

TABLE 1
MOI and method of infection

| | | | |
|---|---|---|---|
| TDV-1 | TDV-2 | TDV-3 | TDV-4 |
| 0.001 | 0.001 | 0.001 | 0.001 |
| Rocking/ | Rocking/ | Rocking/ | Rocking/ |
| Shaking | Shaking | Shaking | Shaking |
| TDV-1 | TDV-2 | TDV-3 | TDV-4 |
| 0.005 | 0.005 | 0.005 | 0.005 |
| Rocking/ | Rocking/ | Rocking/ | Rocking/ |
| Shaking | Shaking | Shaking | Shaking |
| TDV-1 | TDV-2 | TDV-3 | TDV-4 |
| 0.01 | 0.01 | 0.01 | 0.01 |
| Rocking/ | Rocking/ | Rocking/ | Rocking/ |
| Shaking | Shaking | Shaking | Shaking |

Representative average titers obtained for TDV-1 serotype are shown in Table 2 below.

TABLE 2
Average titer values with MOI and method
of infection being rocking/shaking

| | Volume (mL) of | Method of | Average Titer |
|---|---|---|---|
| MOI | Infection Media/cm² | Infection | (log₁₀ PFU/mL) |
| 0.001 | 0.014 | Rocking/Shaking | 8.38 ± 0.28 (a) |
| 0.001 | 0.014 | Rocking/Shaking | 8.46 ± 0.07 (b) |
| 0.005 | 0.029 | Rocking/Shaking | 8.20 ± 0.41 (a) |
| 0.010 | 0.029 | Rocking/Shaking | 8.25 ± 0.37 (a) |

1. Values were the average titers from Day 5, Day 6 and Day 9 samples plus/minus one standard deviation.
(b) Values were the average titers on Day 6 and Day 8 samples plus/minus one standard deviation.

Table 2 shows that high flaviviral titres can be obtained in large-scale flaviviral vaccine production and manufacture at low MOI range irrespective of the volume of infection media used.

Thus, it can be concluded that infection of vero cell monolayer with a low MOI (of less than 0.008, e.g., 0.005 or less) still provides high viral titers in large scale production and manufacture of flaviviral vaccines.

### Example 2-Low MOI Range+Static Infection

This example shows that high viral titers can be obtained in large scale flaviviral vaccine production and manufacture when cells in monolayer are infected with infection media comprising flavivirus at a low MOI less than 0.008, e.g., 0.005 or less and when the method of infection is static.

The same parameters as in Example 1 were used, except that the method of infection was static instead of rocking/shaking. Table 3 below shows representative average titers for TDV-1 serotype.

TABLE 3
Average titer values with MOI and method of infection being static

| | Volume (mL) of | Method of | Average Titer |
|---|---|---|---|
| MOI | Infection Media/cm² | Infection | (log₁₀ PFU/mL) |
| 0.05 | 0.029 | Static | 8.11 ± 0.45 (a) |
| 0.05 | 0.057 | Static | 8.30 ± 0.26 (a) |
| 0.01 | 0.057 | Static | 8.41 ± 0.14 (a) |
| 0.005 | 0.057 | Static | 8.42 ± 0.28 (a) |
| 0.005 | 0.057 | Static | 8.49 ± 0.32 (b) |
| 0.004 | 0.057 | Static | 8.40 ± 0.26 (c) |

(a) Values were the average titers from Day 5, Day 6 and Day 9 samples plus/minus one standard deviation.
(b) Values were the average titers on Day 6 and Day 8 samples plus/minus one standard deviation.
(c) Values were the average titers on Day 6, Day 8 and Day 10 samples plus/minus one standard deviation.

Table 3 shows that high flaviviral titres can still be obtained in large-scale flaviviral vaccine production and manufacture when the infection media comprises flavivirus at low MOI and the method of infection is static rather than rocking/shaking. To further confirm the above findings, using TDV-3 as the representative serotype, a study was performed where the infection volume was fine-tuned from 0.057 mL/cm² to 0.063 mL/cm² while a low MOI was maintained at 0.004. Each TDV-3 daily harvest titer from Day 5 to Day 9 still achieved potency value above 8.0 log 10 PFU/mL.

### Example 2A-Low MOI Infection and Improved Titers

In this experiment three groups were set up according to the table below:

| Groups | MOI | Titers measured at |
|---|---|---|
| 1 | 0.005 | 5-, 6- and 9-days post |
| 2 | 0.05 | infection |

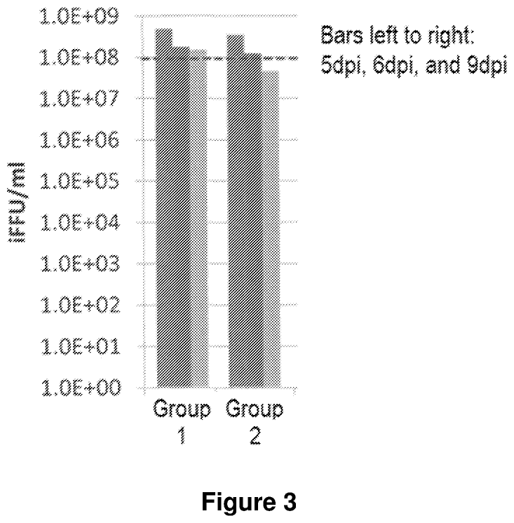

All other parameters were kept constant. The results are shown in FIG. 3. As can be seen in FIG. 3, All titers measured at low MOI (group 1) were above 1.0×10⁸ PFU/mL. Also, the titers of group 2 were above 1.0×10⁸ PFU/mL. However, when comparing groups 1 and 2, the average titer of group 1 i.e., average of day 5, day 6 and day 9 post infection was higher than group 2. This means that low MOI provides consistently high titers up to day 9 post infection, i.e., provides high titers for each harvest day, and that low MOI (group 1) provides on average higher titers as compared to high MOI (group 2)

Another experiment was set up as shown in the Table below:

| Groups | MOI | Titers measured at |
|---|---|---|
| 1 | 0.001 | 5-, 6- and 9-days post |
| 2 | 0.05 | infection |

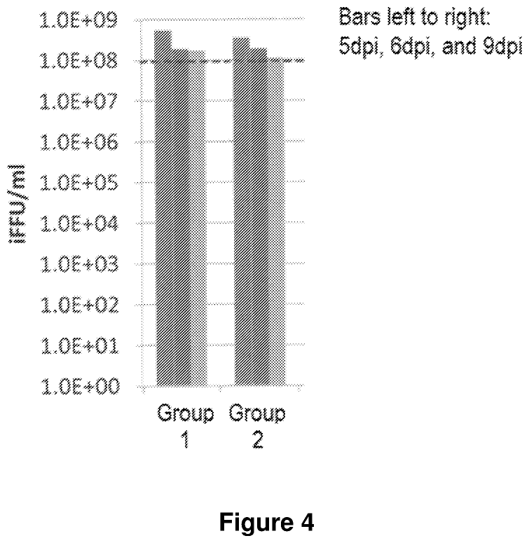

All other parameters were kept constant. The results are shown in FIG. 4. FIG. 4 also shows that that low MOI provides consistently high titers up to day 9 post infection, and that low MOI (group 1) provides on average higher titers as compared to high MOI (group 2)

### Example 3-Media Change and Daily Harvest

This example shows that high viral titers can be obtained in large scale flaviviral vaccine production and manufacture when cells in monolayer are infected with infection media comprising flavivirus at a low MOI (less than 0.008, e.g. 0.005 or less) and when the method of infection is static and when media change is carried out 20 hours before the first harvesting step in combination with at least two or at least three further harvesting steps.

Table 4 below shows representative average titer for TDV-1 serotype. In particular, when infection media comprising flavivirus at low MOI is used in combination with static method of infection and when a media change is carried out 20 hours before the first harvesting step and at least two further harvesting steps are conducted.

TABLE 4
Average titer with MOI, method of infection being static
and media change 20 hours before the first harvest

| | | TDV-1 |
|---|---|---|
| | Method of | Average log₁₀ |
| MOI | Infection | Titer ^{(a)} |
| 0.005 | Static | 8.49 |

^{(a)} Values were the average titers on Day 6 and Day 8 samples plus.

Table 5 below shows representative average titers for TDV-1 serotype. In particular, when infection media comprising flavivirus at low MOI is used in combination with static method of infection and when a media change is carried out 20 hours before the first harvesting step and at least three further harvesting steps are conducted.

TABLE 5
Average titers with MOI, method if infection being static, media
change 20 hours before harvest and three further harvesting steps

| | | TDV-1 | TDV-2 | TDV-3 | TDV-4 |
|---|---|---|---|---|---|
| | | Average | Average | Average | Average |
| | Method of | log₁₀ | log₁₀ | log₁₀ | log₁₀ |
| MOI | Infection | Titer ^{(a)} | Titer ^{(a)} | Titer ^{(a)} | Titer ^{(a)} |
| 0.001 | Static | 8.86 | 8.34 | 8.51 | 7.62 |
| 0.004 | Static | 8.83 | 8.03 | 8.39 | 7.77 |
| 0.008 | Static | 8.76 | 8.08 | 8.25 | 7.83 |

^{(a)}These titer values represent the average value of the individual Day 6, Day 8 and Day 10 harvest titers.

### Example 4—No Substantial Viral Loss when Anion Exchange Chromatography is Performed Before a Tangential Flow Filtration Step

A proof-of-concept study was designed to assess whether a chromatography step can be implemented directly on the clarified and stabilized harvest before an ultrafiltration step without substantial loss of viral titer.

Table 6 below shows the loss of viral titer when a clarified and stabilized harvest is subjected to anion exchange chromatography as compared to the loss of viral titer when a TFF retentate is subjected to anion exchange chromatography. The experiment was replicated with two different chromatography columns.

It was surprisingly found that subjecting the clarified and stabilized harvest directly to anion exchange chromatography before an ultrafiltration step does not lead to substantial loss of titer.

Even more surprisingly, it was found that subjecting the clarified and stabilized harvest directly to anion exchange chromatography leads to higher viral titers as compared to when an ultrafiltration step is carried out immediately before the anion exchange chromatography step.

TABLE 6
Loss of viral titer

| | | Sartobind Q | | Mustang Q | |
|---|---|---|---|---|---|
| | | SingleSep Nano | | Acrodisc (25 mm) | |
| Feed Material | | HC DNA | | HC DNA | |
| Load Volume | | Concentration | log10 Total | Concentration | log10 Total |
| (mL) | Samples | (ng/mL) (a) | Titer (PFU) | (ng/mL) (a) | Titer (PFU) |
| Clarified and | Feed | 1174.79 | 10.46 | 1254.26 | 9.85 |
| stabilized | Flow-through | ≤50 | 10.37 | ≤50 | 9.21 |
| harvest | Reduction | ≥1124 | 0.09 | ≥1204 | 0.64 |
| TFF retentate | Feed | 117.92 | 8.44 | 182.05 | 8.16 |
| | Flow-through | <50 | 7.82 | ≤50 | 6.83 |
| | Reduction | ≥67 | 0.62 | ≥132 | 1.33 |

### Example 5—Complete Process

Each of the four viral serotypes is manufactured separately and constitutes one of the active components of the drug product. The manufacturing process flow for all four serotypes are similar.

The major unit operations are as follows:
1. Vero cell expansion
2. Infection of Vero cells
3. Clarification of daily harvest
4. Daily harvest stabilization with FTA
5. AEX on daily harvest
6. TFF on daily harvest
7. Processing the drug substance to obtain a bulk drug substance
8. Processing the bulk drug substance including filtration of bulk drug substance
9. Processing the bulk drug substance such as freeze and storage of bulk drug substance
10. Formulation of drug product
11. Sterile filtration
12. Filling and Loading
13. Lyophilization and Seal
14. Unloading and Capping
15. Visual Inspection
16. Freezing Final Drug product
17. Labeling and Packaging

The order of the steps above is exemplary and can be adjusted. For example, another variation of these steps can be:
1. Vero cell expansion
2. Infection of Vero cells
3. Clarification of daily harvest
4. Daily harvest stabilization with FTA
5. AEX on daily harvest
6. TFF on daily harvest
7. Processing the drug substance to obtain a bulk drug substance
8. Processing the bulk drug substance such as freeze and storage of bulk drug substance
9. Processing the bulk drug substance such as filtration of bulk drug substance followed by final freeze and storage
10. Formulation of drug product
11. Sterile filtration
12. Filling and Loading
13. Lyophilization and Seal
14. Unloading and Capping
15. Visual Inspection
16. Freezing Final Drug product
17. Labeling and Packaging

### Example 5.1—Vero Cell Expansion

The use of Vero cell line for the manufacture of viral vaccines is widely accepted by regulatory authorities. The continuous mammalian cell line is well characterized with a proven safety profile as it has been used in several approved vaccines. The Vero cell line was shown to support replication of the attenuated dengue vaccine strains, therefore, was used for the manufacture of the master and working virus seeds (MVS) and (WVS) as well as for production of monovalent drug substances.

In this example, a vial of Vero WCB (working cell bank) is thawed in 37° C. water bath. The thawing duration is controlled within 5 minutes. Post-thawed cells are then transferred into the vessels already containing DMEM comprising 3.52 g/L of glucose+10% FBS+4 mM glutamine and cultured at 37° C., 5% CO2, respectively. The media was aseptically mixed and homogenized prior to use. The vessels used for cell expansion in this example included 16×CF10 chambers. This corresponds to a surface are of 101,760 cm2. When the cells are confluent, the cells are dislodged from the surface by TrypLE Select within 40 minutes. During the cell passaging process, allowable cells “dry” duration and maximum duration for cell passaging are defined up to one hour and 8.0 hours, respectively.

A recombinant dissociation trypsin enzyme TrypLE Select was used for dissociation of the cells from the surface of culture vessels. When all the cells are dislodged from the surfaces of the culture vessels, DGM is added to neutralize the activity of TrypLE Select.

### Example 5.2—Infection of Vero Cells and Harvest

After the Vero cells have expanded to sufficient numbers in TDV production vessels, the cells are subsequently infected with monovalent dengue WVS. The infection media used was DMEM without phenol red. The final concentrations of glucose, glutamine and F127 in the infection media were 3.88 g/L, 4.0 mM and 0.1% (w/v), respectively. DMP was prepared by aseptically mixing the sterile liquid components including DMEM, F127 and glutamine, the mixed liquid components was further filtered using Sartopore 2 0.45+0.2 μm filter.

Vero cells are grown to at least 80% confluency on the surface of the culture vessel prior to TDV infection. Vero cells were required to be minimally 75% confluent to attain a cell density of approximately 1.0×10⁵ cells/cm². When the desired cell density has been reached, infection and virus production is performed using DMP.

Monovalent TDV working virus seed (WVS) was used for the infection of the Vero cells. Vero cells with at least 85% confluency in vessels were infected in monolayer with WVS at MOI 0.001 and the infection volume per cm² was 0.057 mL/cm². The method of infection was static, i.e., no rocking mechanism was used for the vessels during the infection process. The infection and virus production parameters; temperature and CO₂ set points were kept at 38° C. and 4%, respectively. The duration of the infection was set at 70 minutes.

Three DPBS wash steps were conducted post infection. Fresh DMP was added into the vessels to facilitate virus production after post-infection DPBS washes.

Six daily harvesting steps were conducted in this example between days 5-10 post infection. Each harvesting step is conducted by collecting the supernantant from the chambers.

28 hours before the first harvesting step, a media change was carried out. After each harvesting step, fresh DMP was added to the cells. The daily harvest volume was up to approximately 12.8 L per daily harvest and up to 76.8 L total harvest volume. All daily harvests achieved greater than 7.0 log 10 PFU/mL with IFA assay.

### Example 5.3—Clarification of the Daily Harvest

Sartopore 2 (0.45+0.2 μm) was used for the clarification of the daily harvest. The throughput capacity of 20.454 mL/cm² (Pmax) was determined for this filter. A conservative filtration flow rate of 800 L/m²/h was chosen for the harvest clarification step with this filter, resulting in a high flow rate, short process time and a reasonable safety margin.

Before clarification, the turbidity of harvests ranged between 1 to 9 nephelometric turbidity units (NTUs). Upon subjecting to the clarification step, a decrease of turbidity (below 1 NTU) is observed. This indicates that the cell debris from the harvest are effectively removed in this clarification step. The average filter backpressure ranged between 0.1 to 0.3 bar. The robustness of the clarification step to reduce the turbidity to the low levels, with relatively low operating backpressure, ensured that the clarification process operated consistently and away from the process boundary.

### Example 5.4—Stabilization of the Harvest

The stabilization buffer was a stock solution of 3×FTA to be added to the harvest to yield a final concentration of 0.2×FTA (0.2% F127, 3% trehalose dihydrate, 0.02% HSA in PBS). A small volume of 3×FTA was used (which was calculated based on one-14th of the total volume of clarified harvest) to yield a concentration of 0.2×FTA.

This is the only time when a buffered excipient composition is added to the harvest during the entire purification process until a drug substance (retentate of TFF) is obtained. This means that the 0.2×FTA clarified and stabilized harvest is the feed for both the anion exchange chromatography step and the tangential flow filtration step. At the end of the tangential flow filtration step, a retentate is obtained, which is the drug substance.

### Example 5.5—Anion Exchange Chromatography

The primary objective of this step is removal of the host cell DNA (HC DNA) while ensuring maximal virus recovery.

AEX column operating in a flow through mode is used in this step. It was found that the stabilized harvest as described above can be directly used as feed material for the AEX column. No buffer exchange or buffer concentration step was needed. Sartobind Q mini column was used in this step that efficiently removed HC DNA with minimal viral loss.

Two 7 mL AEX capsule at the scale of 16×CF10 were used in this example. The flow rate was 230 mL/min.

The DBC of Sartobind Q 7 mL membrane ranged from 2.15 to 3.69 mg of DNA per 1 mL of Membrane bed volume (MV).

AEX step was sufficient to reduce the amount of host cell DNA to values below the specification of ≤50 ng/mL.

### Example 5.6—Tangential Flow Filtration

A TFF membrane with molecular weight cut-off (MWCO) of 300 kDa polyethersulfone (PES) was was used in this example. The pore size is smaller than the size of a dengue virus particle which is approximately 50 nm and thus is able to retain the virus. The feed flow rate of 300 LMH was used.

The flow through from the AEX could be directly implemented as feed composition for the TFF step.

The TFF rententate obtained is the drug substance.

### Example 5.7-Processing the Drug Substance to Obtain a Bulk Drug Substance

Three flushing steps of the TFF membrane were conducted to obtain a bulk drug substance.

The flushing buffer was 1×FTA. However, to compensate for the trehalose dihydrate passage through the TFF membrane, the buffer flush composition comprised of 27% trehalose dihydrate concentration, to yield a 15% trehalose dihydrate concentration in the monovalent bulk drug substance.

The analyzed data of the FTA content of the bulk drug substance, for all serotypes, produced during the production scale runs was close to the theoretical value of 1×FTA concentration.

### Example 5.8—Processing the Bulk Drug Substance Such as Filtration of the Bulk Drug Substance

In this step, the filtration of the bulk drug substance was conducted immediately on the bulk drug substance for TDV-3 and TDV-4.

Sartopore 2 filter was also adopted for the filtration of the bulk drug substance. This is a hydrophilic polyethersulfone membrane with pore size 0.45+0.2 μm.

The filter size of 525.0 cm² was used for the situation where concentrated individual harvests are pooled prior to filtration, and 87.6 cm² for the situation where each daily harvest is filtered separately. The closest membrane area sizes available from the filter manufacturer are 0.1 m² and 150 cm² respectively.

The analyzed data of the FTA content of the filtered bulk drug substance, for all serotypes, produced during the production scale runs was close to the theoretical value of 1×FTA concentration.

### Example 5.9—Freeze and Storage of Bulk Drug Substance

After the filtration step, of TDV-3 and TDV-4, the bulk drug substances are frozen and stored. While for TDV-1 and TDV-2, the bulk drug substance of each harvesting day was frozen and thawed on the day of obtaining the last bulk drug substance from the last harvesting step and then all bulk drug substances were pooled and then subjected to a filtration step followed by final freezing and storing. The objective of the freezing step is to ensure the drug substance potency and other quality attributes are maintained in order for the material to be used for drug product formulation.

6L FFtp bags were used for this purpose as they offer the possibility of utilizing various freeze/thaw options, while maintaining the same product contact surface. This ensures sufficient ease and robustness in transportation and product compatibility at various fill volumes.

The bags of bulk drug substance are stored frozen until they are thawed for use in the formulation of the drug product.

### Example 5.10—Formulation of Drug Product

After the bulk drug substance was frozen, the next step was formulation of the bulk drug product. This included bulk drug substance thawing, and mixing the thawed bulk drug substance (i.e., for each of TDV-1, TDV-2, TDV-3 and TDV-4) with a formulation buffer to obtain a bulk drug product.

The formulation buffer comprised two buffers, the first excipient buffer (FEB) and a second excipient buffer (SEB). Qualitatively, the components of each of FEB and SEB are the same. However, quantitatively, the components of each of FEB and SEB are different.

After sterile filtration of the drug product (step 5.11), it was filled and loaded in vials (step 5.12) and directly moved to the next step.

### Example 5.13—Lyophilization and Seal

An exemplary lyophilization cycle is shown in Table 8 below:

TABLE 7
Exemplary lyophilization cycle

| | | Normal Operating Ranges | |
|---|---|---|---|
| Cycle Step | Parameter | (NOR) | |
| Initial shelf | Temp (° C.) | 5° C. ± 2° C. | |
| temperature during | | | |
| loading | | | |
| Equilibration | Temp (° C.) | 5° C. ± 2° C. | |
| | Time (min) | 60 ± 5 | min |
| | Pressure (mbar) | Atmospheric pressure | |
| Freezing rate | Rate | 0.5° C./min (95-105 | |
| | (° C./time) | min to reach −45° | |
| | | C. ± 2° C.) | |
| Freezing hold | Pressure (mbar) | Atmospheric pressure | |
| | Temp (° C.) | −45° C. ± 2° C. | |
| | Time (min) | 140 ± 5 min | |
| | Pressure (mbar) | Atmospheric pressure | |
| Primary drying | Rate ( | 0.5° C./min (31-41 | |
| ramp | ° C./time) | min to reach −27° | |
| | | C. ± 2° C.) | |
| | Time taken to | 30-105 | min |
| | reach chamber | | |
| | pressure for | | |
| | primary drying | | |
| Primary drying | Temp (° C.) | −27° C. ± 2° C. | |
| hold | Time (min) | 2880 ± 5 | min |
| | Pressure (mbar) | 0.024 ± 0.005 | mbar |
| | Pressure rise | Pressure increase to max. | |
| | test | 0.044 mbar in 1 min | |
| Secondary drying | Rate | 0.1° C./min (515-525 | |
| ramp | (° C./time) | min to reach | |
| | | 25° C. ± 2° C.) | |
| Secondary drying | Temp (° C.) | 25° C. ± 2° C. | |
| hold | Time (min) | 285 ± 5 | min |
| | Pressure (mbar) | 0.024 ± 0.005 | mbar |
| | Pressure rise | Pressure increase to max. | |
| | test | 0.044 mbar in 1 min | |
| Vial cooling temperature | Temp (° C.) | 3° C.-7° C. | |
| (for 2° C.-8° C. storage | | | |
| in freeze-dryer) | | | |
| Chamber pressure before | Pressure (mbar) | −0.125 to −0.075 | mbar rel. |
| stoppering | | | |
| Stoppering pressure | Pressure | ≥6.5 | N/cm2 |
| | (N/cm²) | | |
| Vial stoppering time | Time (sec) | 60 | s |

After Unloading and Capping (step 5.14)- and Visual Inspection (step 5.15), the final drug product is frozen (step 5.16) and finally labelled and packaged in step 5.17.

### Example 6—Complete Process

Each of the four viral serotypes is manufactured separately and constitutes one of the active components of the drug product. The manufacturing process flow for all four serotypes (drug substance) are similar, with differences in individual serotypes driven by titer and capacity needs.

The major unit operations are as follows:
1. Vero cell expansion
2. Infection of Vero cells
3. Clarification of daily harvest
4. Daily harvest stabilization with FTA
5. AEX on daily harvest
6. TFF on daily harvest
7. Processing the drug substance to obtain a bulk drug substance
8. Processing the bulk drug substance such as filtration of bulk drug substance
9. Processing the bulk drug substance such as freeze and storage of bulk drug substance
10. Formulation of drug product
11. Sterile filtration
12. Filling and Loading
13. Lyophilization and Seal
14. Unloading and Capping
15. Visual Inspection
16. Freezing Final Drug product
17. Labeling and Packaging
   The order of the steps above is exemplary and can be adjusted. For example, another variation of these steps can be:
1. Vero cell expansion
2. Infection of Vero cells
3. Clarification of daily harvest
4. Daily harvest stabilization with FTA
5. AEX on daily harvest
6. TFF on daily harvest
7. Processing the drug substance to obtain a bulk drug substance
8. Processing the bulk drug substance such as freeze and storage of bulk drug substance
9. Processing the bulk drug substance such as filtration of bulk drug substance followed by final freeze and storage
10. Formulation of drug product
11. Sterile filtration
12. Filling and Loading
13. Lyophilization and Seal
14. Unloading and Capping
15. Visual Inspection
16. Freezing Final Drug product
17. Labeling and Packaging

### Example 6.1—Vero Cell Expansion

A vial of Vero WCB is thawed in 37° C. water bath. In this example, the thawing duration is controlled within 8 minutes. Post-thawed cells are then transferred into the vessels already containing DMEM comprising 4.0 g/L of glucose+5% FBS+4.5 mM glutamine and cultured at 38° C., 4% CO2, respectively. Phenol red was excluded from the media and visual microscopic check was employed. The media used was mixed together and filtered to maintain microbial control prior to use. The vessels used for cell expansion in this example include 4×CF40 chambers. This corresponds to a surface are of 101,760 cm². When the cells are confluent, the cells are dislodged from the surface by TrypLE Select within 20 minutes. During the cell passaging process, allowable cells “dry” duration and maximum duration for cell passaging are defined up to 30 minutes and 5.0 hours respectively.

A recombinant dissociation trypsin enzyme TrypLE Select was used for dissociation of the cells from the surface of culture vessels was used as in Example 5.1. However, in this example, prior to adding TrypLE Select, the cells are washed with DPBS to remove residual FBS. When all the cells are dislodged from the surfaces of the culture vessels, DGM is added to neutralize the activity of TrypLE Select.

### Example 6.2—Infection of Vero Cells

After the Vero cells have expanded to sufficient numbers in production vessels, the cells are subsequently infected with monovalent dengue WVS (working virus seed). The infection media (DMP) was DMEM without phenol red. The final concentrations of glucose, glutamine and F68 in the infection media were 3 g/L, 4.5 mM and 0.2% (w/v), respectively. In this example, DMP was filtered using Sartopore 2 0.45+0.2 μm filter with aseptically mixing the liquid components.

In this example, Vero cells are grown to at least 90% confluency on the surface of the culture vessel prior to infection. Here, Vero cells were required to be minimally 90% confluent to attain a cell density of approximately 2×10⁵ cells/cm². When the desired cell density has been reached, infection and virus production is performed using DMP.

Monovalent TDV working virus seed (WVS) was used for the infection of the Vero cells. Vero cells with at least 90% confluency were infected in monolayer with WVS at MOI 0.0014 and the infection volume per cm² was 0.014 mL/cm². Manual rocking of CF10s during the infection process was performed. In this example, a DPBS washing step was conducted prior to infection, as part of a series of dilution steps. The infection and virus production parameters; temperature and CO₂ set points were kept at 37° C. and 5%, respectively. The duration of the infection was set at 90 minutes.

Three DPBS wash steps were also conducted post infection. Fresh DMP was added to facilitate virus production after post-infection DPBS washes.

Three daily harvesting steps were conducted in this example between days 5-7 post infection. Harvesting step included collecting the supernatant from the chambers. 12 hours before the first harvesting step, a media change was carried out. After each harvesting step, fresh DMP was added to the cells. The daily harvest volume was up to approximately 12.8 L per daily harvest and up to 38.4 L total harvest volume. All daily harvests achieved greater than 7.0 log 10 PFU/mL with IFA assay.

### Example 6.3—Clarification of the Daily Harvest

Sartopore 2 (0.45+0.2 μm) was used for the clarification of the daily harvest. The throughput capacity of 20.454 mL/cm² (Pmax) was determined for this filter. The filtration flow rate used in this study was 300 mL/min (equal to 1200 L/m²/h [LMH]) for a Sartopore 2 150 cm² filter. A conservative filtration flow rate of 800 L/m²/h was chosen for the harvest clarification step with this filter, resulting in a high flow rate, short process time and a reasonable safety margin.

Before clarification, harvests ranged between 1 to 9 nephelometric turbidity units (NTUs). Upon subjecting to the clarification step, a decrease of turbidity (below 1 NTU) is observed. This indicates that the cell debris from the harvest are effectively removed in this clarification step. The average filter backpressure ranged between 0.2 to 0.4 bar. The robustness of the clarification step to reduce the turbidity to the low levels, with relatively low operating backpressure, ensured that the clarification process operated consistently and away from the process boundary.

### Example 6.4—Stabilization of the Harvest

The stabilization buffer was a stock solution of 3×FTA to be added to the harvest to yield a final concentration of 0.2×FTA (0.2% F123, 3% trehalose dihydrate, 0.02% HSA in PBS). A smaller volume of 3×FTA was used (which was calculated based on one-14th of the total volume of clarified harvest) to yield a concentration of 0.2×FTA.

This is the only time when a buffered excipient composition is added to the harvest during the entire purification process until a drug substance is obtained. This means that the 0.2×FTA harvest is the feed for both the anion exchange chromatography step and the tangential flow filtration step. At the end of the tangential flow filtration step, a retentate is obtained, which is the drug substance.

### Example 6.5—Anion Exchange Chromatography

The primary objective of this step is removal of the host cell DNA (HC DNA) while ensuring maximal virus recovery.

AEX column operating in a flow through mode is used in this step. It was found that the stabilized harvest as described above can be directly used as feed material for the AEX column. No buffer exchange or buffer concentration step was needed. Mustang Q column was used in this step that efficiently removed HC DNA with minimal viral loss.

Two 7 mL AEX capsule were used in this example. The flow rate was 230 mL/min. AEX step was sufficient to reduce the amount of host cell DNA to values below the specification of ≤50 ng/ml.

The DBC of Mustang Q membrane ranged from 2.15 to 3.69 mg of DNA per 1 mL of Membrane bed volume (MV).

### Example 6.6—Tangential Flow Filtration

A TFF membrane with molecular weight cut-off (MWCO) of 100 kDa (Hydrosart) were was used in this example. The pore size is smaller than the size of a dengue virus particle which is approximately 50 nm and should thus be able to retain the virus. The feed flow rate of 180 LMH was used.

The flow through from the AEX could be directly implemented as feed composition for the TFF step.

The TFF rententate obtained is the drug substance.

### Example 6.7—Processing the Drug Substance

Three flushing steps of the TFF membrane were conducted to obtain a bulk drug substance.

The flushing buffer was 1×FTA. However, to compensate for the trehalose dihydrate passage through the TFF membrane, the buffer flush composition comprised of 27% trehalose dihydrate concentration, to yield a 15% trehalose dihydrate concentration in the monovalent bulk drug substance.

The analyzed data of the FTA content of the bulk drug substance, for all serotypes, produced during the production scale runs was close to the theoretical value of 1×FTA concentration.

### Example 6.8—Processing the Bulk Drug Substance

The filtration of the bulk drug substance was conducted immediately on the bulk drug substance for TDV-3 and TDV-4.

Sartopore 2 filter was also adopted for the filtration of the bulk drug substance. This is a hydrophilic polyethersulfone membrane with pore size 0.45+0.2 μm.

The filter size of 525.0 cm2 was used when concentrated individual harvests are pooled prior to filtration, and 87.6 cm2 when each daily harvest is filtered separately. The closest membrane area sizes available from the filter manufacturer are 0.1 m² and 150 cm² respectively.

### Example 6.9—Freeze and Storage of Bulk Drug Substance

After the filtration step, of TDV-3 and TDV-4, the bulk drug substances are frozen and stored. While for TDV-1 and TDV-2, the bulk drug substance of each harvesting day was frozen and thawed on the day of obtaining the last bulk drug substance from the last harvesting step and then all bulk drug substances were pooled and then subjected to a filtration step followed by final freezing and storing. The objective of the freezing step is to ensure the drug substance potency and other quality attributes are maintained in order for the material to be used for drug product formulation.

Bulk drug substance is stored frozen until thawed for use in the formulation of the drug product using a plate freezer.

### Example 6.10—Formulation of Drug Product

After the bulk drug substance was frozen, the next step was formulation of the bulk drug product. This included bulk drug substance thawing, and mixing the thawed bulk drug substance (i.e., for each of TDV-1, TDV-2, TDV-3 and TDV-4) with a formulation buffer to obtain a bulk drug product.

The formulation buffer comprised two buffers, the first excipient buffer (FEB) and a second excipient buffer (SEB). Qualitatively, the components of each of FEB and SEB are the same. However, quantitatively, the components of each of FEB and SEB are different.

After sterile filtration of the drug product (step 6.11), it was filled and loaded in vials (step 6.12) and directly moved to the next step.

### Example 6.13—Lyophilization and Seal

An exemplary lyophilization cycle is shown in Table 11 below:

TABLE 8
Exemplary lyophilization cycle

| | | Normal Operating Ranges | |
|---|---|---|---|
| Cycle Step | Parameter | (NOR) | |
| Initial shelf temperature | Temp (° C.) | 5° C. ± 2° C. | |
| during loading | | | |
| Equilibration | Temp (° C.) | 5° C. ± 2° C. | |
| | Time (min) | 70 ± 10 | min |
| | Pressure (mbar) | Atmospheric pressure | |
| Freezing rate | Rate | 0.5° C./min | |
| | (° C./time) | (95-105 min to reach −45° | |
| | | C. ± 2° C.) | |
| | Pressure (mbar) | Atmospheric pressure | |
| Freezing hold | Temp (° C.) | −45° C. ± 2° C. | |
| | Time (min) | 150 ± 10 | min |
| | Pressure (mbar) | Atmospheric pressure | |
| Primary drying ramp | Rate | 0.5° C./min (31-41 min | |
| | (° C./time) | to reach −27° | |
| | | C. ± 2° C.) | |
| | Time taken to | 30-105 | min |
| | reach chamber | | |
| | pressure for | | |
| | primary drying | | |
| Primary drying hold | Temp (° C.) | −27° C. ± 2° C. | |
| | Time (min) | 2890 ± 10 | min |
| | Pressure (mbar) | 0.024 ± 0.005 | mbar |
| | Pressure rise | Pressure increase to max. | |
| | test | 0.044 mbar in 1 min | |
| Secondary drying ramp | Rate | 0.1° C./min (515-525 | |
| | (° C./time) | min to reach 25° C.+2° C.) | |
| Secondary drying hold | Temp (° C.) | 25° C. ± 2° C. | |
| | Time (min) | 295 ± 10 | min |
| | Pressure (mbar) | 0.024 ± 0.005 | mbar |
| | Pressure rise | Pressure increase to max. | |
| | test | 0.044 mbar in 1 min | |
| Vial cooling temperature | Temp (° C.) | 3° C.-7° C. | |
| (for 2° C.-8° C. storage | | | |
| in freeze-dryer) | | | |
| Chamber pressure before | Pressure (mbar) | −0.125 to −0.075 mbar rel. | |
| stoppering | | | |
| Stoppering pressure | Pressure (N/cm²) | ≥6.5 | N/cm2 |
| Vial stoppering time | Time (sec) | 60 | s |

After Unloading and Capping (step 6.14) and Visual Inspection (step 6.15), the final drug product is frozen in step 6.16 and finally in step 6.17-labelled and packaged.

### Example 7: pH Fluctuation and Virus Yield

The optimum pH for the entire purification process was in the range from 7.6 to 8.1. In order to test, whether pH fluctuations affect virus yields, an experiment was set up with two groups as follows:
Group A: pH was maintained within 7.6 to 8.1 but allowed to fluctuate by 0.5 units
Group B: pH was maintained within 7.6 to 8.1 but allowed to fluctuate by less than 0.5 units (0.3 units in this group).

All other conditions that would not allow fair comparison between the two groups were kept constant.

The results are provided in Table 9 below:

| | Group A (pH | Group B (pH |
|---|---|---|
| | value at each | value at each |
| Process step | process step) | process step) |
| Harvest 1 | 7.8 | 7.8 |
| Harvest 2 | 7.8 | 7.8 |
| Harvest 3 | 8.1 | 7.8 |
| Harvest 4 | 7.9 | 7.7 |
| Harvest 5 | 7.8 | 7.8 |
| Harvest 6 | 8.0 | 7.8 |
| Post stabilisation 1 | 8.1 | 7.7 |
| Post stabilisation 2 | 7.7 | 7.7 |
| Post stabilisation 3 | 7.8 | 7.6 |
| Post stabilisation 4 | 7.7 | 7.8 |
| Post stabilisation 5 | 7.8 | 7.9 |
| Post stabilisation 6 | 7.7 | 7.7 |
| Post AEX 1 | 7.7 | 7.8 |
| Post AEX 2 | 7.8 | 7.7 |
| Post AEX 3 | 7.8 | 7.9 |
| Post AEX 4 | 7.7 | 7.9 |
| Post AEX 5 | 7.7 | 7.7 |
| Post AEX 6 | 7.9 | 7.7 |
| After TFF 1 | 7.7 | 7.6 |
| After TFF 2 | 7.7 | 7.6 |
| After TFF 3 | 7.7 | 7.6 |
| After TFF 4 | 7.7 | 7.7 |
| After TFF 5 | 7.7 | 7.6 |
| After TFF 6 | 7.7 | 7.6 |
| Post-Thaw 1 | 7.6 | 7.7 |
| Post-Thaw 2 | 7.6 | 7.6 |
| Post-Thaw 3 | 7.7 | 7.6 |
| Post-Thaw 4 | 7.6 | 7.7 |
| Post-Thaw 5 | 7.7 | 7.7 |
| Post-Thaw 6 | 7.7 | 7.7 |
| Bulk drug substance | 7.7 | 7.7 |

The allowed fluctuation in pH units according to Table 9, above,
and yields obtained in each group are shown below in Table 10

| | Allowed | Virus | Virus | Total |
|---|---|---|---|---|
| | fluctuation | Yield in | Yield in | number of |
| Group | in pH | grams | mg/cm2 | CF10 flasks |
| Group A | 0.5 units | 23,951 | 40.5 | 93 |
| Group B | 0.3 units | 25,052 | 42.4 | 93 |

It was surprisingly found that the yield of the virus can be improved significantly by not only maintaining the pH throughout the process steps at a given pH range (here 7.6 to 8.1) but also by ensuring that the pH is not allowed to fluctuate by more than 0.3 units. The calculation of virus yield in mg/cm² is conducted as follows:

### Group A

$$Total number of CF 10 flasks ( 93 ) \times area of each CF 10 flask ( 6360 cm 2 ) = total area \qquad ( i )$$

$$Total virus yield in milligrams ( 23951000 g ) / Total area obtained from step ( i ) , \\ above ( 591480 cm 2 ) = 40.5 mg / cm 2 \qquad ( ii )$$

### Group B

$$Total number of CF 10 flasks ( 93 ) \times area of each CF 10 flask ( 6360 cm 2 ) = total area \qquad ( i )$$

$$Total virus yield in milligrams ( 23951000 g ) / Total area obtained from step ( i ) , \\ above ( 591480 cm 2 ) = 40.5 mg / cm 2 \qquad ( ii )$$

Further, the application refers to the following items:

Item 1: A method for large-scale flaviviral vaccine production and manufacture comprising the following sequential steps:
(i) providing cells in growth media,
(ii) infecting the cells of step (i) with infection media comprising flavivirus at a low MOI,
(iii) harvesting to obtain a harvest,
(iv) processing the harvest of step (iii) to obtain a processed harvest,
(v) purifying the processed harvest of step (iv),
wherein step (v) comprises at least one chromatography step.
   Item 1A: A method for large-scale Flaviviral vaccine production and manufacture comprising the following sequential steps:
(i) providing cells in growth media,
(ii) infecting the cells of step (i) with infection media comprising flavivirus,
(iii) harvesting to obtain a harvest,
(iv) processing the harvest of step (iii) to obtain a processed harvest,
(v) purifying the processed harvest of step (iv), wherein step (v) comprises at least one chromatography step,
   wherein the pH throughout steps (iii) to (v) is maintained at a range from 7.6 to 8.1, and wherein the difference between
(a) the maximum pH occurring at any time point throughout steps (iii) to (v), and
(b) the minimum pH occurring at any time point throughout steps (iii) to (v),
is no greater than 0.4 units.
   Item 1B: The method of item 1A, wherein the difference between item 1A (a) and item 1A (b) is no greater than 0.3 units.
   Item 1C: The method of item 1A, wherein the method results in higher viral yield as compared to the same method with the same process steps but allowing a difference between item 1A (a) and item 1A (b) to be greater than 0.4 units.
   Item 1D: The method of item 1B, wherein the method results in higher viral yield as compared to the same method with the same process steps but allowing a difference between between item 1A (a) and item 1A (b) to be greater than 0.3 units.
   Item 1E: The method of item 1C or 1D, wherein the increase in viral yield is at least about 1 mg/cm² such as 1.5 mg/cm².
   Item 2: The method of item 1, 1A, 1B, 1C, 1D, or 1E, wherein the method of infection in step (ii) is static, and/or the cells are infected in monolayer.
   Item 3: The method of items item 1, 1A, 1B, 1C, 1D, 1E or 2, wherein the low MOI is less than 0.008.
   Item 4: The method of item 3, wherein the low MOI is 0.005 or less.
   Item 5: The method of any one of item 1, 1A, 1B, 1C, 1D, 1E, or 2 to 4, wherein harvesting comprises a first harvesting step and optionally at least one further harvesting step.
   Item 6: The method of any one of item 1, 1A, 1B, 1C, 1D, 1E or 2 to 5, wherein harvesting in step (iii) comprises collecting the supernatant from the cells obtained in step (ii).
   Item 7: The method of item 6, wherein collecting the supernatant is immediately followed by adding fresh media.
   Item 8: The method of any one of items 5-7, wherein at least 12 to 30 hours before the first harvesting step, a media change is carried out.
   Item 9: The method of item 8, wherein the media change comprises discarding the supernatant and adding fresh media.
   Item 10: The method of any one of item 5 to 9, wherein the first harvesting step is carried out on day 2, day 3, day 4, day 5, day 6, day 7, day 8, day 9, or day 10 post infection.
   Item 11: The method of any one of items 5 to 10, wherein the at least one further harvesting step comprises up to nine, preferably five further harvesting steps.
   Item 12: The method of item 11, wherein the interval between each harvesting step is at least 20 to 30 hours.
   Item 13: The method of any one of the preceding items, wherein step (i) comprises expanding cells in growth media.
   Item 14: The method of item 13, wherein the growth media does not comprise a non-ionic surfactant.
   Item 15: The method of any one of the preceding items, wherein the cells are adherent cells.
   Item 16: The method of any one of the preceding items, wherein the cells comprise abundant flaviviral receptors.
   Item 17: The method of any one of the preceding items, wherein the cells comprise abundant dengue virus receptors.
   Item 18: The method of item 15, wherein the cells are selected from a group consisting of Madin-Darby Canine Kidney cells, monkey cell lines pMK, Vero cells, and human cell lines HEK 293, MRC 5, Per.C6, PMK, and WI-38.
   Item 19: The method of item 18, wherein the cells are Vero cells.
   Item 20: The method of any one of 2 to 19, wherein the infection media of step (ii) covers the cell monolayer.
   Item 21: The method of any one of the preceding items, wherein step (ii) comprises incubating the cells for 50 to 250 minutes, 60 to 200 minutes, or 70 to 150 minutes.
   Item 22: The method of item 21, wherein the cells are incubated at a temperature range between 36 to 39° C.
   Item 23: The method of any one of the preceding items, wherein step (ii) is immediately followed by one, two, three or four wash step(s).
   Item 24: The method of item 23, wherein the cells are washed with PBS, DPBS, or TBS.
   Item 25: The method of any one of the preceding items, wherein step (iv) comprises clarification of the harvest.
   Item 26: The method of item 25, wherein clarification of the harvest comprises one or more depth filtration steps.
   Item 27: The method of item 26, wherein the one or more depth filtration steps comprise a filter comprising a pore size of 0.1 μm to 1 μm.
   Item 28: The method of any one of items 26 to 27, wherein the one or more depth filtration steps comprise a heterogenous double layer filter.
   Item 29: The method of any one of items 25 to 28, wherein immediately after clarification, the harvest is frozen.
   Item 30: The method of item 6, wherein the harvest is immediately frozen after collection of the supernatant.
   Item 31: The method of item 30, wherein the clarification is carried out on pooled harvests obtained from a first harvesting step and at least one further harvesting step.
   Item 32: The method of any one of the preceding items, wherein step (iv) comprises stabilization of the harvest.
   Item 33: The method of item 32, wherein stabilization comprises adding a stabilization buffer to the harvest or to the clarified harvest.
   Item 34: The method of item 33, wherein the stabilization buffer comprises a sugar, a surfactant, and a protein.
   Item 35: The method of any one of the preceding items, wherein step (v) comprises the following sequential steps:
(v-a) ion exchange chromatography to obtain a purified harvest,
(v-b) ultrafiltration.
   Item 36: The method of item 35, wherein the ion exchange chromatography is anion exchange chromatography.
   Item 37: The method of items 35 to 36, wherein step (v-a) is carried out on stabilized harvest.
   Item 38: The method of any one of items 35 to 7, wherein ultrafiltration of step (v-b) is tangential flow filtration.
   Item 39: The method of any one of items 35 to 38, wherein the purified harvest obtained after step (v-a) comprises 50 ng/ml or lower host cell DNA.
   Item 40: The method of any one of items 35 to 39, wherein a drug substance is obtained after the ultrafiltration of step (v-b)
   Item 41: The method of any one of items 35 to 40, further comprising step (vi) of processing the drug substance preferably comprising at least one flushing step of the ultrafiltration membrane with a flushing buffer to obtain a composition comprising a buffer flush and drug substance.
   Item 42: The method of item 41, wherein the composition comprising the drug substance and the buffer flush is a bulk drug substance.
   Item 43: The method of any one of items 41 to 42, wherein the flushing buffer comprises a sugar, a surfactant, and a protein.
   Item 44: The method of any one of items 35 to 45, wherein the volume of the drug substance of step (v-b) is two- to ten-folds lower than the volume obtained after step (v-a).
   Item 45: The method of any one of items 42 to 44, wherein
step (iii) comprises a first harvesting step to obtain a first harvest,
step (iv) comprises processing the first harvest obtained in step (iii) comprising clarification and stabilization of the harvest to obtain a first clarified and stabilized harvest,
step (v) comprises purification of the clarified and stabilized harvest obtained in step (iv) comprising
   (v-a) at least one chromatography step such as an anion exchange chromatography to obtain a first purified harvest, and
   (v-b) ultrafiltration such as tangential flow filtration on the purified harvest of step (v-a) to obtain a first drug substance, and
step (vi) at least one flushing step of the ultrafiltration membrane with a flushing buffer to obtain a composition comprising a buffer flush and drug substance, which is the first bulk drug substance
   wherein all of steps (iii) to (vi) are performed on the same day to obtain the first bulk drug substance.
   Item 46: The method of item 45, wherein 20-30 hours after the first harvesting step of (iii), a second harvesting step is conducted, and steps (iv) to (vi) are repeated on the same day to obtain a second bulk drug substance.
   Item 47: The method of item 46, wherein a total of up to ten harvesting steps are conducted to obtain ten bulk drug substances, preferably a total of up to six harvesting steps are conducted to obtain six bulk drug substances, wherein the interval between each harvesting step is 20-30 hours.
   Item 48: The method of any one of item 1, 1A, 1B, 1C, 1D, or 1E or 2 to 47, wherein the flavivirus is a live, attenuated dengue virus selected from a group consisting of dengue serotype 1 (DENV-1) such as a dengue 2/1 chimera, dengue serotype 2 (DENV-2), dengue serotype 3 (DENV-3) such as a dengue 2/3 chimera and dengue serotype 4 (DENV-4) such as a dengue 2/4 chimera.
   Item 49: The method of any one of item 1, item 1A, 1B, 1C, 1D, or 1E or 2 to 48, wherein the flavivirus is a live, attenuated dengue virus selected from a group consisting of TDV-1 represented by SEQ ID NO: 1 and/or 2, TDV-2 represented by SEQ ID NO: 3 and/or 4, TDV-3 represented by SEQ ID NO: 5 and/or 6, and TDV-4 represented by SEQ ID NO: 7 and/or 8.
   Item 50: The method of any one of items 42 to 47, comprising step (vii) of processing the bulk drug substance comprising
(vii-a) filtering the bulk drug substance to obtain a filtered bulk drug substance,
(vii-b) freezing the filtered bulk drug substance obtained from step (vii-a) to obtain a frozen bulk drug substance,
   preferably wherein steps (vii-a) and (vii-b) are conducted on the same day as step (vi).
   Item 51: The method of item 50, comprising step (viii) of storing the frozen bulk drug substance, obtained from step (vii-b), preferably wherein step (viii) is conducted on the same day as step (vii-a) and (vii-b).
   Item 52: The method of any one of items 50 and 51, wherein the flavivirus is a live, attenuated dengue virus selected from a group consisting of dengue serotype 3 (DENV-3) such as a dengue 2/3 chimera, and dengue serotype 4 (DENV-4) such as a dengue 2/4 chimera.
   Item 53: The method of any one of items 50 to 52, wherein the flavivirus is a live, attenuated dengue virus selected from a group consisting of TDV-3 represented by SEQ ID NO: 5 and/or 6, and TDV-4 represented by SEQ ID NO: 7 and/or 8.
   Item 54: The method of any one of items 42 to 47, comprising step (vii) of processing the bulk drug substance comprising
(vii-a) freezing the bulk drug substances obtained from the first harvesting step and at least one further harvesting step to obtain frozen bulk drug substances, preferably from a total of up to ten harvesting steps to obtain ten frozen bulk drug substances, more preferably, from a total of six harvesting steps to obtain six frozen bulk drug substances,
(vii-b) thawing the bulk drug substances of step (vii-a) to obtain thawed bulk drug substances,
(vii-c) pooling the thawed bulk drug substances of step (vii-b) to obtain pooled bulk drug substances.
(vii-d) subjecting the pooled bulk drug substances obtained from step (vii-c) to a filtration step to obtain filtered drug substances
(vii-e) freezing the filtered drug substances from step (vii-d) to obtain frozen bulk drug substances,
   preferably wherein steps (vii-a) to (vii-e) are conducted on the same day as step (vi).
   Item 55: The method of item 54, comprising step (viii) of storing the frozen bulk drug substances obtained from step (vii-e), preferably wherein step (viii) is conducted on the same day as step (vii-a) to (vii-e).
   Item 56: The method of item 54 to 55, wherein the flavivirus is a live, attenuated dengue virus selected from a group consisting of dengue serotype 1 (DENV-1) such as a dengue 2/1 chimera, and dengue serotype 2 (DENV-2).
   Item 57: The method of any one of items 54 to 56, wherein the flavivirus is a live, attenuated dengue virus selected from a group consisting of TDV-1 represented by SEQ ID NO: 1 and/or 2, TDV-2 represented by SEQ ID NO: 3 and/or 4.
   Item 58: The method of any one of items 51-53 or 55-57, further comprising step (ix) of formulation of drug product.
   Item 59: The method of item 58, wherein formulation of drug product comprises the following sequential steps:
(ix-a) bulk drug substances thawing,
(ix-b) Mixing the thawed bulk drug substances of step (ix-a) with the formulation buffer.
   Item 60: The method of item 59, wherein the formulation buffer comprises a first excipient buffer (FEB) and a second excipient buffer (SEB).
   Item 61: The method of item 60, wherein the FEB and SEB each comprise four salts, a sugar, a non-ionic surfactant, and a protein.
   Item 62: The method of any one of items 59 to 61, wherein step (ix-a) comprises thawing two, three, or four bulk drug substances each comprising a distinct dengue serotype selected from a group consisting of a live, attenuated dengue virus selected from a group consisting of dengue serotype 1 (DENV-1) such as a dengue 2/1 chimera, dengue serotype 2 (DENV-2), dengue serotype 3 (DENV-3) such as a dengue 2/3 chimera, dengue serotype 4 (DENV-4) such as a dengue 2/4 chimera . . .
   Item 63: The method of any one of items 59 to 62, wherein step (ix-a) comprises thawing two, three, or four bulk drug substances each comprising a distinct dengue serotype selected from a group consisting of TDV-1 represented by SEQ ID NO: 1 and/or 2, TDV-2 represented by SEQ ID NO: 3 and/or 4, TDV-3 represented by SEQ ID NO: 5 and/or 6, and TDV-4 represented by SEQ ID NO: 7 and/or 8 . . .
   Item 64: The method of item 62 or 63, wherein immediately after step (ix-a) and before step (ix-b), the two, three, or four thawed bulk drug substances are mixed.
   Item 65: The method of any one of items 62 to 64, wherein the viral vaccine is divalent, trivalent or tetravalent, preferably wherein the viral vaccine is the tetravalent dengue vaccine TAK-003.
   Item 66: The method of any one of items 48 to 65, wherein the live, attenuated dengue-2 virus serotype is in the form of DENV-2 16681 derived DEN-2 PDK-53 variant with a triple mutation at NS1-53, at 5′NCR-57 and at NS3-250 such that the amino acid position 250 of the NS3 protein contains a valine residue, and wherein the chimeras have said DEN-2 PDK-53 genome as viral backbone and one or more structural protein genes encoding capsid, premembrane/membrane or envelope of said DEN-2 PDK-53 genome or combinations thereof replaced with one or more corresponding structural protein genes from DEN-1, DEN-3 or DEN-4.
   Item 67: The method of any one of items 48 to 66, wherein the dengue 2/1 chimera has two further mutations such that the amino acid position 116 of the NS2A protein contains a leucine residue and the amino acid position 92 of the NS2B protein contains an aspartic acid residue, wherein the live, attenuated dengue-2 virus serotype has two further mutations such that the amino acid position 52 of the prM protein contains a glutamic acid residue and the amino acid position 412 of the NS5 protein contains a valine residue, wherein the dengue-2/3 chimera has said DEN-2 PDK-53 genome as viral backbone and has the prM-E gene (nt-457 to -2373) of said DEN-2 PDK-53 genome replaced with the corresponding prM-E gene from wild-type DEN-3 16562 and wherein said dengue-2/3 chimera has one further mutation at said corresponding prM-E gene from wild-type DEN-3 16562 such that the amino acid position 223 of the E protein contains a serine residue, and wherein the dengue-2/4 chimera has two further mutations such that the amino acid position 66 of the NS2A protein contains a glycine residue and the amino acid position 21 of the NS4A protein contains a valine residue and wherein the dengue-2/4 chimera is a mixed genotype with respect to amino acid position 99 of the NS2A protein containing an arginine or a lysine residue.
   Item 68: The method of any one of items 48 to 67, wherein the dengue-2/1 chimera has said DEN-2 PDK-53 genome as viral backbone and has the prM-E gene (nt-457 to -2379) of said DEN-2 PDK-53 genome replaced with the corresponding prM-E gene from wild-type DEN-1 16007, wherein the dengue-2/3 chimera has said DEN-2 PDK-53 genome as viral backbone and has the prM-E gene (nt-457 to -2373) of said DEN-2 PDK-53 genome replaced with the corresponding prM-E gene from wild-type DEN-3 16562, and wherein the dengue-2/4 chimera has said DEN-2 PDK-53 genome as viral backbone and has the prM-E gene (nt-457 to -2379) of said DEN-2 PDK-53 replaced with the corresponding prM-E gene from wild-type DEN-4 1036.
   Item 69: The method of any one of items 48 to 68, wherein the live, attenuated dengue-2 virus serotype is represented by a polynucleotide of SEQ ID NO: 3, or a polypeptide of SEQ ID NO: 4; the dengue 2/1 chimera having nonstructural proteins from a modified live, attenuated dengue-2 virus serotype and structural proteins from a dengue-1 virus serotype, represented by a polynucleotide of SEQ ID NO: 1, or a polypeptide of SEQ ID NO: 2; the dengue 2/3 chimera having nonstructural proteins from a modified live, attenuated dengue-2 virus serotype and structural proteins from a dengue-3 virus serotype, represented by a polynucleotide of SEQ ID NO: 5, or a polypeptide of SEQ ID NO: 6; and the dengue 2/4 chimera having nonstructural proteins from a modified live, attenuated dengue-2 virus serotype and structural proteins from a dengue-4 virus serotype, represented by a polynucleotide of SEQ ID NO: 7, or a polypeptide of SEQ ID NO: 8.
   Item 70: The method of any one of preceding items, wherein the method provides for high viral titres.
   Item 71: The method of any one of preceding items, wherein the method provides higher average viral titres as compared to a method comprising the same process steps but wherein at step (ii), the cells are infected with infection media comprising a flavivirus at high MOI, preferably wherein high MOI refers to MOI greater than 0.008.
   Item 72: The method of any one of preceding items, wherein the method provides higher average viral titres as compared to a method comprising the same process steps but wherein at step (ii), the cells are infected with infection media comprising a flavivirus at high MOI, and a media change is carried 12-30 hours before the first harvesting step, preferably wherein high MOI refers to MOI greater than 0.008.
   Item 73: The method of any one of items 70, wherein the high viral titres are titres greater than 7.0 log 10 PFU/mL, preferably greater than 7.5 log 10 PFU/mL.
   Item 74: The method of any one of the preceding items, wherein large-scale comprises production cultures of surface area 35,000 cm² or more.
   Item 75: The method of item 74, wherein large-scale comprises production cultures of surface area 50,000 cm² or more.
   Item 76: The method of items 74 or 75, wherein large-scale comprises production cultures of surface area 100,000 cm² or more.
   Item 77: The method of any one of the preceding items, wherein the viral vaccine is a live, attenuated tetravalent dengue vaccine, preferably TAK-003.
   Item 78: The method of any one of the preceding items, wherein the pH throughout steps (iii) to (v) is maintained at a range from 7.6 to 8.1, and wherein the difference between
(a) the maximum pH occurring at any time point throughout steps (iii) to (v), and
(b) the minimum pH occurring at any time point throughout steps (iii) to (v),
is no greater than 0.4 units.
   Item 79: The method of any one of the items 45 to 77, wherein the pH throughout steps (iii) to (vi) is maintained at a range from 7.6 to 8.1, and wherein the difference between
(a) the maximum pH occurring at any time point throughout steps (iii) to (vi), and
(b) the maximum pH occurring at any time point throughout steps (iii) to (vi),
is no greater than 0.4 units.
   Item 80: The method of any one of the items 50 to 77, wherein the pH throughout steps (iii) to

(vii) is maintained at a range from 7.6 to 8.1, and wherein the difference between
(a) the maximum pH occurring at any time point throughout steps (iii) to (vii), and
(b) the maximum pH occurring at any time point throughout steps (iii) to (vii),
is no greater than 0.4 units.
   Item 81: A method for large-scale Flaviviral vaccine production and manufacture comprising the following sequential steps:
(i) providing cells in growth media,
(ii) infecting the cells of step (i) with infection media comprising flavivirus,
(iii) harvesting to obtain a harvest,
(iv) processing the harvest of step (iii) to obtain a processed harvest,
(v) purifying the processed harvest of step (iv), wherein step (v) comprises at least one chromatography step,
   wherein the pH throughout steps (iii) to (v) is maintained at a range from 7.6 to 8.1, and wherein the difference between
(a) the maximum pH occurring at any time point throughout steps (iii) to (v), and
(b) the minimum pH occurring at any time point throughout steps (iii) to (v),
is no greater than 0.4 units.
   Item 82: The method of item 78, wherein the difference between item 78 (a) and 78 (b) is no greater than 0.3 units.
   Item 83: The method of item 79, wherein the difference between item 79 (a) and 79 (b) is no greater than 0.3 units.
   Item 84: The method of item 80, wherein the difference between item 80 (a) and 80 (b) is no greater than 0.3 units.
   Item 85: The method of item 81, wherein the difference between item 81 (a) and 81 (b) is no greater than 0.3 units.
   Item 86: The method of item 78, wherein the method results in higher viral yield as compared to the same method with the same process steps but allowing a difference between item 78 (a) and item 78 (b) to be greater than 0.4 units.
   Item 87: The method of item 79, wherein the method results in higher viral yield as compared to the same method with the same process steps but allowing a difference between item 79 (a) and item 79 (b) to be greater than 0.4 units.
   Item 88: The method of item 80, wherein the method results in higher viral yield as compared to the same method with the same process steps but allowing a difference between item 80 (a) and item 80 (b) to be greater than 0.4 units.
   Item 89: The method of item 81, wherein the method results in higher viral yield as compared to the same method with the same process steps but allowing a difference between item 81 (a) and item 81 (b) to be greater than 0.4 units.
   Item 90: The method of any one of items 81, wherein the method results in higher viral yield as compared to the same method with the same process steps but allowing a difference between item 81 (a) and item 81 (b) to be greater than 0.3 units.
   Item 91: The method of item 82, wherein the method results in higher viral yield as compared to the same method with the same process steps but allowing a difference between item 82 (a) and item 82 (b) to be greater than 0.3 units.
   Item 92: The method of item 83, wherein the method results in higher viral yield as compared to the same method with the same process steps but allowing a difference between item 83 (a) and item 83 (b) to be greater than 0.3 units.
   Item 93: The method of item 84, wherein the method results in higher viral yield as compared to the same method with the same process steps but allowing a difference between item 84 (a) and item 84 (b) to be greater than 0.3 units.
   Item 94: The method of any one of items 86 to 93, wherein the increase in viral yield is at least about 1 mg/cm² such as 1.5 mg/cm².
   Item 95: The viral composition obtained by the method according to any one of the previous items.
   Item 96: The viral vaccine obtained by the method according to items 65 to 88.
   Item 97: A pharmaceutical package comprising the viral vaccine obtained by the method according to any one of the preceding items.

1: A method for large-scale flaviviral vaccine production and manufacture comprising the following sequential steps:
(i) providing cells in growth media,
(ii) infecting the cells of step (i) with infection media comprising flavivirus at a low MOI,
(iii) harvesting to obtain a harvest,
(iv) processing the harvest of step (iii) to obtain a processed harvest,
(v) purifying the processed harvest of step (iv),
wherein step (v) comprises at least one chromatography step.

2: The method of claim 1, wherein the method of infection in step (ii) is static, and/or the cells are infected in monolayer.

3: The method of claim 1 or 2, wherein the low MOI is less than 0.008.

4: The method of claim 3, wherein the low MOI is 0.005 or less.

5: The method of any one of claims 1 to 4, wherein harvesting comprises a first harvesting step and optionally at least one further harvesting step.

6: The method of any one of claims 1 to 5, wherein harvesting in step (iii) comprises collecting a supernatant from the cells obtained in step (ii).

7: The method of claim 6, wherein collecting the supernatant is immediately followed by adding fresh media.

8: The method of any one of claims 5-7, wherein at least 12 to 30 hours before the first harvesting step, a media change is carried out.

9: The method of claim 8, wherein the media change comprises discarding the supernatant and adding fresh media.

10: The method of any one of claims 5 to 9, wherein the first harvesting step is carried out on day 2, day 3, day 4, day 5, day 6, day 7, day 8, day 9, or day 10 post infection.

11: The method of any one of claims 5 to 10, wherein the at least one further harvesting step comprises up to nine, preferably five further harvesting steps.

12: The method of claim 11, wherein the interval between each harvesting step is at least 20 to 30 hours.

13: The method of any one of the preceding claims, wherein step (i) comprises expanding cells in growth media.

14: The method of claim 13, wherein the growth media does not comprise a non-ionic surfactant.

15: The method of any one of the preceding claims, wherein the cells are adherent cells.

16: The method of any one of the preceding claims, wherein the cells comprise abundant flaviviral receptors.

17: The method of any one of the preceding claims, wherein the cells comprise abundant dengue virus receptors.

18: The method of claim 15, wherein the cells are selected from a group consisting of Madin-Darby Canine Kidney cells, monkey cell lines pMK, Vero cells, and human cell lines HEK 293, MRC 5, Per.C6, PMK, and WI-38.

19: The method of claim 18, wherein the cells are Vero cells.

20: The method of any one of claims 2 to 19, wherein the infection media of step (ii) covers the cell monolayer.

21: The method of any one of the preceding claims, wherein step (ii) comprises incubating the cells for 50 to 250 minutes, 60 to 200 minutes, or 70 to 150 minutes.

22: The method of claim 21, wherein the cells are incubated at a temperature range between 36 to 39° C.

23: The method of any one of the preceding claims, wherein step (ii) is immediately followed by one, two, three or four wash step(s).

24: The method of claim 23, wherein the cells are washed with PBS, DPBS, or TBS.

25: The method of any one of the preceding claims, wherein step (iv) comprises clarification of the harvest.

26: The method of claim 25, wherein clarification of the harvest comprises one or more depth filtration steps.

27: The method of claim 26, wherein the one or more depth filtration steps comprise employing a filter comprising a pore size of 0.1 μm to 1 μm.

28: The method of any one of claims 26 to 27, wherein the one or more depth filtration steps comprise employing a heterogenous double layer filter.

29: The method of any one of claims 25 to 28, wherein immediately after clarification, the harvest is frozen.

30: The method of claim 6, wherein the harvest is immediately frozen after collection of the supernatant.

31: The method of claim 30, wherein the clarification is carried out on pooled harvests obtained from a first harvesting step and at least one further harvesting step.

32: The method of any one of the preceding claims, wherein step (iv) comprises stabilization of the harvest.

33: The method of claim 32, wherein stabilization comprises adding a stabilization buffer to the harvest or to the clarified harvest.

34: The method of claim 33, wherein the stabilization buffer comprises a sugar, a surfactant, and a protein.

35: The method of any one of the preceding claims, wherein step (v) comprises the following sequential steps:
(v-a) ion exchange chromatography to obtain a purified harvest,
(v-b) ultrafiltration.

36: The method of claim 35, wherein the ion exchange chromatography is anion exchange chromatography.

37: The method of claims 35 to 36, wherein step (v-a) is carried out on clarified and stabilized harvest.

38: The method of any one of claims 35 to 7, wherein ultrafiltration of step (v-b) is tangential flow filtration.

39: The method of any one of claims 35 to 38, wherein the purified harvest obtained after step (v-a) comprises 50 ng/mL or lower host cell DNA.

40: The method of any one of claims 35 to 39, wherein a drug substance is obtained after the ultrafiltration of step (v-b)

41: The method of any one of claims 35 to 40, further comprising step (vi) of processing the drug substance preferably comprising at least one flushing step of the ultrafiltration membrane with a flushing buffer to obtain a composition comprising a buffer flush and drug substance.

42: The method of claim 41, wherein the composition comprising the drug substance and the buffer flush is a bulk drug substance.

43: The method of any one of claims 41 to 42, wherein the flushing buffer comprises a sugar, a surfactant, and a protein.

44: The method of any one of claims 35 to 45, wherein the volume of the drug substance of step (v-b) is two- to ten-folds lower than the volume of the purified harvest obtained after step (v-a).

45: The method of any one of claims 42 to 44, wherein
step (iii) comprises a first harvesting step to obtain a first harvest,
step (iv) comprises processing the first harvest obtained in step (iii) comprising clarification and stabilization of the harvest to obtain a first clarified and stabilized harvest,
step (v) comprises purification of the clarified and stabilized harvest obtained in step (iv) comprising
(v-a) at least one chromatography step such as an anion exchange chromatography to obtain a first purified harvest, and
(v-b) ultrafiltration such as tangential flow filtration on the purified harvest of step
(v-a) to obtain a first drug substance, and
step (vi) at least one flushing step of the ultrafiltration membrane with a flushing buffer to obtain a composition comprising a buffer flush and drug substance, which is the first bulk drug substance
wherein all of steps (iii) to (vi) are performed on the same day to obtain the first bulk drug substance.

46: The method of claim 45, wherein 20-30 hours after the first harvesting step of (iii), a second harvesting step is conducted, and steps (iv) to (vi) are repeated on the same day to obtain a second bulk drug substance.

47: The method of claim 46, wherein a total of up to ten harvesting steps are conducted to obtain ten bulk drug substances, preferably a total of up to six harvesting steps are conducted to obtain six bulk drug substances, wherein the interval between each harvesting step is 20-30 hours.

48: The method of any one of claims 1 to 47, wherein the flavivirus is a live, attenuated dengue virus selected from a group consisting of dengue serotype 1 (DENV-1) such as a dengue 2/1 chimera, dengue serotype 2 (DENV-2), dengue serotype 3 (DENV-3) such as a dengue 2/3 chimera and dengue serotype 4 (DENV-4) such as a dengue 2/4 chimera.

49: The method of any one of claims 1 to 48, wherein the flavivirus is a live, attenuated dengue virus selected from a group consisting of TDV-1 represented by SEQ ID NO: 1 and/or 2, TDV-2 represented by SEQ ID NO: 3 and/or 4, TDV-3 represented by SEQ ID NO: 5 and/or 6, and TDV-4 represented by SEQ ID NO: 7 and/or 8.

50: The method of any one of claims 42 to 47, comprising step (vii) of processing the bulk drug substance comprising
(vii-a) filtering the bulk drug substance to obtain a filtered bulk drug substance,
(vii-b) freezing the filtered bulk drug substance obtained from step (vii-a) to obtain a frozen bulk drug substance,
preferably wherein steps (vii-a) and (vii-b) are conducted on the same day as step (vi).

51: The method of claim 50, comprising step (viii) of storing the frozen bulk drug substance, obtained from step (vii-b), preferably wherein step (viii) is conducted on the same day as step (vii-a) and (vii-b).

52: The method of any one of claims 50 and 51, wherein the flavivirus is a live, attenuated dengue virus selected from a group consisting of dengue serotype 3 (DENV-3) such as a dengue 2/3 chimera, and dengue serotype 4 (DENV-4) such as a dengue 2/4 chimera.

53: The method of any one of claims 50 to 52, wherein the flavivirus is a live, attenuated dengue virus selected from a group consisting of TDV-3 represented by SEQ ID NO: 5 and/or 6, and TDV-4 represented by SEQ ID NO: 7 and/or 8.

54: The method of any one of claims 42 to 47, comprising step (vii) of processing the bulk drug substance comprising
(vii-a) freezing the bulk drug substances obtained from the first harvesting step and at least one further harvesting step to obtain frozen bulk drug substances, preferably from a total of up to ten harvesting steps to obtain ten frozen bulk drug substances, more preferably, from a total of six harvesting steps to obtain six frozen bulk drug substances,
(vii-b) thawing the bulk drug substances of step (vii-a) to obtain thawed bulk drug substances,
(vii-c) pooling the thawed bulk drug substances of step (vii-b) to obtain pooled bulk drug substances.
(vii-d) subjecting the pooled bulk drug substances obtained from step (vii-c) to a filtration step to obtain filtered drug substances
(vii-e) freezing the filtered drug substances from step (vii-d) to obtain frozen bulk drug substances,
preferably wherein steps (vii-a) to (vii-e) are conducted on the same day as step (vi).

55: The method of claim 54, comprising step (viii) of storing the frozen bulk drug substances obtained from step (vii-e), preferably wherein step (viii) is conducted on the same day as step (vii-a) to (vii-e).

56: The method of claims 54 to 55, wherein the flavivirus is a live, attenuated dengue virus selected from a group consisting of dengue serotype 1 (DENV-1) such as a dengue 2/1 chimera, and dengue serotype 2 (DENV-2).

57: The method of any one of claims 54 to 56, wherein the flavivirus is a live, attenuated dengue virus selected from a group consisting of TDV-1 represented by SEQ ID NO: 1 and/or 2, TDV-2 represented by SEQ ID NO: 3 and/or 4.

58: The method of any one of claim 51-53 or 55-57, further comprising step (ix) of formulation of drug product.

59: The method of claim 58, wherein formulation of drug product comprises the following sequential steps:
(ix-a) thawing the bulk drug substances,
(ix-b) Mixing the thawed bulk drug substances of step (ix-a) with the formulation buffer.

60: The method of claim 59, wherein the formulation buffer comprises a first excipient buffer (FEB) and a second excipient buffer (SEB).

61: The method of claim 60, wherein the FEB and SEB each comprise four salts, a sugar, a non-ionic surfactant, and a protein.

62: The method of any one of claims 59 to 61, wherein step (ix-a) comprises thawing two, three, or four bulk drug substances each comprising a distinct dengue serotype selected from a group consisting of a live, attenuated dengue virus selected from a group consisting of dengue serotype 1 (DENV-1) such as a dengue 2/1 chimera, dengue serotype 2 (DENV-2), dengue serotype 3 (DENV-3) such as a dengue 2/3 chimera, dengue serotype 4 (DENV-4) such as a dengue 2/4 chimera.

63: The method of any one of claims 59 to 62, wherein step (ix-a) comprises thawing two, three, or four bulk drug substances each comprising a distinct dengue serotype selected from a group consisting of TDV-1 represented by SEQ ID NO: 1 and/or 2, TDV-2 represented by SEQ ID NO: 3 and/or 4, TDV-3 represented by SEQ ID NO: 5 and/or 6, and TDV-4 represented by SEQ ID NO: 7 and/or 8.

64: The method of claim 62 or 63, wherein immediately after step (ix-a) and before step (ix-b), the two, three, or four thawed bulk drug substances are mixed.

65: The method of any one of claims 62 to 64, wherein the viral vaccine is divalent, trivalent or tetravalent, preferably wherein the viral vaccine is a tetravalent dengue vaccine TAK-003.

66: The method of any one of claims 48 to 65, wherein the live, attenuated dengue-2 virus serotype is in the form of DENV-2 16681 derived DEN-2 PDK-53 variant with a triple mutation at NS1-53, at 5′NCR-57 and at NS3-250 such that the amino acid position 250 of the NS3 protein contains a valine residue, and wherein the chimeras have said DEN-2 PDK-53 genome as viral backbone and one or more structural protein genes encoding capsid, premembrane/membrane or envelope of said DEN-2 PDK-53 genome or combinations thereof replaced with one or more corresponding structural protein genes from DEN-1, DEN-3 or DEN-4.

67: The method of any one of claims 48 to 66, wherein the dengue 2/1 chimera has two further mutations such that the amino acid position 116 of the NS2A protein contains a leucine residue and the amino acid position 92 of the NS2B protein contains an aspartic acid residue, wherein the live, attenuated dengue-2 virus serotype has two further mutations such that the amino acid position 52 of the prM protein contains a glutamic acid residue and the amino acid position 412 of the NS5 protein contains a valine residue, wherein the dengue-2/3 chimera has said DEN-2 PDK-53 genome as viral backbone and has the prM-E gene (nt-457 to -2373) of said DEN-2 PDK-53 genome replaced with the corresponding prM-E gene from wild-type DEN-3 16562 and wherein said dengue-2/3 chimera has one further mutation at said corresponding prM-E gene from wild-type DEN-3 16562 such that the amino acid position 223 of the E protein contains a serine residue, and wherein the dengue-2/4 chimera has two further mutations such that the amino acid position 66 of the NS2A protein contains a glycine residue and the amino acid position 21 of the NS4A protein contains a valine residue and wherein the dengue-2/4 chimera is a mixed genotype with respect to amino acid position 99 of the NS2A protein containing an arginine or a lysine residue.

68: The method of any one of claims 48 to 67, wherein the dengue-2/1 chimera has said DEN-2 PDK-53 genome as viral backbone and has the prM-E gene (nt-457 to -2379) of said DEN-2 PDK-53 genome replaced with the corresponding prM-E gene from wild-type DEN-1 16007, wherein the dengue-2/3 chimera has said DEN-2 PDK-53 genome as viral backbone and has the prM-E gene (nt-457 to -2373) of said DEN-2 PDK-53 genome replaced with the corresponding prM-E gene from wild-type DEN-3 16562, and wherein the dengue-2/4 chimera has said DEN-2 PDK-53 genome as viral backbone and has the prM-E gene (nt-457 to -2379) of said DEN-2 PDK-53 replaced with the corresponding prM-E gene from wild-type DEN-4 1036.

69: The method of any one of claims 48 to 68, wherein the live, attenuated dengue-2 virus serotype is represented by a polynucleotide of SEQ ID NO: 3, or a polypeptide of SEQ ID NO: 4; the dengue 2/1 chimera having nonstructural proteins from a modified live, attenuated dengue-2 virus serotype and structural proteins from a dengue-1 virus serotype, represented by a polynucleotide of SEQ ID NO: 1, or a polypeptide of SEQ ID NO: 2; the dengue 2/3 chimera having nonstructural proteins from a modified live, attenuated dengue-2 virus serotype and structural proteins from a dengue-3 virus serotype, represented by a polynucleotide of SEQ ID NO: 5, or a polypeptide of SEQ ID NO: 6; and the dengue 2/4 chimera having nonstructural proteins from a modified live, attenuated dengue-2 virus serotype and structural proteins from a dengue-4 virus serotype, represented by a polynucleotide of SEQ ID NO: 7, or a polypeptide of SEQ ID NO: 8.

70: The method of any one of preceding claims, wherein the method provides for high viral titres.

71: The method of any one of preceding claims, wherein the method provides higher average viral titres as compared to a method comprising the same process steps but wherein at step (ii), the cells are infected with infection media comprising a flavivirus at high MOI, preferably wherein high MOI refers to MOI greater than 0.008.

72: The method of any one of preceding claims, wherein the method provides higher average viral titres as compared to a method comprising the same process steps but wherein at step (ii), the cells are infected with infection media comprising a flavivirus at high MOI, and a media change is carried 12-30 hours before the first harvesting step, preferably wherein high MOI refers to MOI greater than 0.008.

73: The method of any one of claim 70, wherein the high viral titres are titres greater than 7.0 log 10 PFU/mL, preferably greater than 7.5 log 10 PFU/mL.

74: The method of any one of the preceding claims, wherein large-scale comprises production cultures of surface area 35,000 cm² or more.

75: The method of claim 74, wherein large-scale comprises production cultures of surface area 50,000 cm² or more.

76: The method of claim 74 or 75, wherein large-scale comprises production cultures of surface area 100,000 cm² or more.

77: The method of any one of the preceding claims, wherein the viral vaccine is a live, attenuated tetravalent dengue vaccine, preferably TAK-003.

78: The method of any one of the preceding claims, wherein the pH throughout steps (iii) to (v) is maintained at a range from 7.6 to 8.1, and wherein the difference between
(a) the maximum pH occurring at any time point throughout steps (iii) to (v), and
(b) the minimum pH occurring at any time point throughout steps (iii) to (v),
is no greater than 0.4 units.

79: The method of any one of the claims 45 to 77, wherein the pH throughout steps (iii) to (vi) is maintained at a range from 7.6 to 8.1, and wherein the difference between
(a) the maximum pH occurring at any time point throughout steps (iii) to (vi), and
(b) the maximum pH occurring at any time point throughout steps (iii) to (vi),
is no greater than 0.4 units.

80: The method of any one of the claims 50 to 77, wherein the pH throughout steps (iii) to (vii) is maintained at a range from 7.6 to 8.1, and wherein the difference between
(a) the maximum pH occurring at any time point throughout steps (iii) to (vii), and
(b) the maximum pH occurring at any time point throughout steps (iii) to (vii),
is no greater than 0.4 units.

81: A method for large-scale Flaviviral vaccine production and manufacture comprising the following sequential steps:
(i) providing cells in growth media,
(ii) infecting the cells of step (i) with infection media comprising flavivirus,
(iii) harvesting to obtain a harvest,
(iv) processing the harvest of step (iii) to obtain a processed harvest,
(v) purifying the processed harvest of step (iv), wherein step (v) comprises at least one chromatography step,
wherein the pH throughout steps (iii) to (v) is maintained at a range from 7.6 to 8.1, and wherein the difference between
(a) the maximum pH occurring at any time point throughout steps (iii) to (v), and
(b) the minimum pH occurring at any time point throughout steps (iii) to (v),
is no greater than 0.4 units.

82: The method of claim 78, wherein the difference between claims 78 (a) and 78 (b) is no greater than 0.3 units.

83: The method of claim 79, wherein the difference between claims 79 (a) and 79 (b) is no greater than 0.3 units.

84: The method of claim 80, wherein the difference between claims 80 (a) and 80 (b) is no greater than 0.3 units.

85: The method of claim 81, wherein the difference between claims 81 (a) and 81 (b) is no greater than 0.3 units.

86: The method of claim 78, wherein the method results in higher viral yield as compared to the same method with the same process steps but allowing a difference between claim 78 (a) and claim 78 (b) to be greater than 0.4 units.

87: The method of claim 79, wherein the method results in higher viral yield as compared to the same method with the same process steps but allowing a difference between claim 79 (a) and claim 79 (b) to be greater than 0.4 units.

88: The method of claim 80, wherein the method results in higher viral yield as compared to the same method with the same process steps but allowing a difference between claim 80 (a) and claim 80 (b) to be greater than 0.4 units.

89: The method of claim 81, wherein the method results in higher viral yield as compared to the same method with the same process steps but allowing a difference between claim 81 (a) and claim 81 (b) to be greater than 0.4 units.

90: The method of any one of claim 81, wherein the method results in higher viral yield as compared to the same method with the same process steps but allowing a difference between claim 81 (a) and claim 81 (b) to be greater than 0.3 units.

91: The method of claim 82, wherein the method results in higher viral yield as compared to the same method with the same process steps but allowing a difference between claim 82 (a) and claim 82 (b) to be greater than 0.3 units.

92: The method of claim 83, wherein the method results in higher viral yield as compared to the same method with the same process steps but allowing a difference between claim 83 (a) and claim 83 (b) to be greater than 0.3 units.

93: The method of claim 84, wherein the method results in higher viral yield as compared to the same method with the same process steps but allowing a difference between claim 84 (a) and claim 84 (b) to be greater than 0.3 units.

94: The method of any one of claims 86 to 93, wherein the increase in viral yield is at least about 1 mg/cm² such as 1.5 mg/cm².

95: The method of any one of claims 81 to 94, wherein the flavivirus is a live, attenuated dengue virus selected from a group consisting of dengue serotype 1 (DENV-1), dengue serotype 2 (DENV-2), dengue serotype 3 (DENV-3), and dengue serotype 4 (DENV-4).

96: The method of claim 48, 62, or 95, wherein the flavivirus is a live, attenuated dengue virus selected from a group consisting of dengue 2/1 chimera, dengue serotype 2, dengue 2/3 chimera and a dengue 2/4 chimera.claim 95: The viral composition obtained by the method according to any one of the previous claims.

97: The viral vaccine obtained by the method according to claims 65 to 94.

98: A pharmaceutical package comprising the viral vaccine obtained by the method according to any one of the preceding claims.


SEQUENCE LISTING (8 sequences)

SEQ ID NO: 1 - DNA, 10723 bp, synthetic construct
  source 1..10723: mol_type = other DNA; note = chimeric dengue serotype 2/1
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacgcgtgggggagagccgcatatgatagttagcaagcaggaaagaggaaagtcacttttgttcaagacctctgcaggtgtcaacatgtgcaccctcattgcgatggatttgggagagttgtgtgaggacacgatgacctacaaatgcccccggatcactgaggcggaaccagatgacgttgactgttggtgcaatgccacggacacatgggtgacctatggaacgtgctctcaaactggcgaacaccgacgagacaaacgttccgtcgcattggccccacacgtggggcttggcctagaaacaagagccgaaacgtggatgtcctctgaaggtgcttggaaacagatacaaaaagtagagacttgggctctgagacatccaggattcacggtgatagccctttttctagcacatgccataggaacatccatcacccagaaagggatcattttcattttgctgatgctggtaacaccatctatggccatgcgatgcgtgggaataggcaacagagacttcgtggaaggactgtcaggagcaacatgggtggatgtggtactggagcatggaagttgcgtcaccaccatggcaaaaaacaaaccaacactggacattgaactcttgaagacggaggtcacaaaccctgcagttctgcgtaaattgtgcattgaagctaaaatatcaaacaccaccaccgattcgagatgtccaacacaaggagaagccacactggtggaagaacaagacgcgaactttgtgtgccgacgaacgttcgtggacagaggctggggcaatggctgtgggctattcggaaaaggtagtctaataacgtgtgccaagtttaagtgtgtgacaaaactagaaggaaagatagttcaatatgaaaacctaaaatattcagtgatagtcaccgtccacactggagatcagcaccaggtgggaaatgagactacagaacatggaacaactgcaaccataacacctcaagctcctacgtcggaaatacagctgaccgactacggaacccttacattagattgttcacctaggacagggctagattttaacgagatggtgttgctgacaatgaaagaaagatcatggcttgtccacaaacaatggttcctagacttaccactgccttggacctctggggcttcaacatcccaagagacttggaacagacaagatttactggtcacatttaagacagctcatgcaaagaagcaggaagtagtcgtactaggatcacaagaaggagcaatgcacactgcgctgactggagcgacagaaatccaaacgtcaggaacgacaacaattttcgcaggacacctaaaatgcagactaaaaatggacaaactaactttaaaagggatgtcatatgtgatgtgcacaggctcattcaagttagagaaagaagtggctgagacccagcatggaactgttctggtgcaggttaaatatgaaggaacagacgcaccatgcaagattcccttttcgacccaagatgagaaaggagcaacccagaatgggagattaataacagccaaccccatagtcactgacaaagaaaaaccagtcaatattgaggcagaaccaccctttggtgagagctacatcgtggtaggagcaggtgaaaaagctttgaaactaagctggttcaagaaaggaagcagcatagggaaaatgtttgaagcaactgcccgaggagcacgaaggatggccattctgggagacaccgcatgggacttcggttctataggaggagtgttcacgtctatgggaaaactggtacaccaggtttttggaactgcatatggagttttgtttagcggagtttcttggaccatgaaaataggaatagggattctgctgacatggctaggattaaattcaaggaacacgtccctttcgatgatgtgcatcgcagccggcattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccctaccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagatcaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccgaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 2 - protein, 3391 aa, synthetic construct
  source 1..3391: note = chimeric dengue serotype 2/1
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRGGEPHMIVSKQERGKSLLFKTSAGVNMCTLIAMDLGELCEDTMTYKCPRITEAEPDDVDCWCNATDTWVTYGTCSQTGEHRRDKRSVALAPHVGLGLETRAETWMSSEGAWKQIQKVETWALRHPGFTVIALFLAHAIGTSITQKGIIFILLMLVTPSMAMRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTTATITPQAPTSEIQLTDYGTLTLDCSPRTGLDFNEMVLLTMKERSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSTQDEKGATQNGRLITANPIVTDKEKPVNIEAEPPFGESYIVVGAGEKALKLSWFKKGSSIGKMFEATARGARRMAILGDTAWDFGSIGGVFTSMGKLVHQVFGTAYGVLFSGVSWTMKIGIGILLTWLGLNSRNTSLSMMCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTLPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEDQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 3 - DNA, 10723 bp, synthetic construct
  source 1..10723: mol_type = other DNA; note = dengue serotype 2
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacacgtaacggagaaccacacatgatcgtcagcagacaagagaaagggaaaagtcttctgtttaaaacagaggttggcgtgaacatgtgtaccctcatggccatggaccttggtgaattgtgtgaagacacaatcacgtacgagtgtccccttctcaggcagaatgagccagaagacatagactgttggtgcaactctacgtccacgtgggtaacttatgggacgtgtaccaccatgggagaacatagaagagaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgatcttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccatcacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccatttggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccgtattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 4 - protein, 3391 aa, synthetic construct
  source 1..3391: note = dengue serotype 2
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRNGEPHMIVSRQEKGKSLLFKTEVGVNMCTLMAMDLGELCEDTITYECPLLRQNEPEDIDCWCNSTSTWVTYGTCTTMGEHRREKRSVALVPHVGMGLETRTETWMSSEGAWKHVQRIETWILRHPGFTMMAAILAYTIGTTHFQRALIFILLTAVTPSMTMRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAVFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 5 - DNA, 10717 bp, synthetic construct
  source 1..10717: mol_type = other DNA; note = chimeric dengue serotype 2/3
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggaccattaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagatctgcaggcatgatcattatgctgattccaacagtgatggcgttccatttaaccacgcgtgatggagagccgcgcatgattgtggggaagaatgaaagaggaaaatccctacttttcaagacagcctctggaatcaacatgtgcacactcatagctatggatctgggagagatgtgtgatgacacggtcacttacaaatgcccccacattaccgaagtggagcctgaagacattgactgctggtgcaaccttacatcgacatgggtgacttatggaacatgcaatcaagctggagagcatagacgcgataagagatcagtggcgttagctccccatgttggcatgggactggacacacgcactcaaacctggatgtcggctgaaggagcttggagacaagtcgagaaggtagagacatgggcccttaggcacccagggtttaccatactagccctatttcttgcccattacataggcacttccttgacccagaaagtggttatttttatactattaatgctggttaccccatccatgacaatgagatgtgtaggagtaggaaacagagattttgtggaaggcctatcgggagctacgtgggttgacgtggtgctcgagcacggtgggtgtgtgactaccatggctaagaacaagcccacgctggacatagagcttcagaagaccgaggccacccaactggcgaccctaaggaagctatgcattgagggaaaaattaccaacataacaaccgactcaagatgtcccacccaaggggaagcgattttacctgaggagcaggaccagaactacgtgtgtaagcatacatacgtggacagaggctggggaaacggttgtggtttgtttggcaagggaagcttggtgacatgcgcgaaatttcaatgtttagaatcaatagagggaaaagtggtgcaacatgagaacctcaaatacaccgtcatcatcacagtgcacacaggagaccaacaccaggtgggaaatgaaacgcagggagtcacggctgagataacaccccaggcatcaaccgctgaagccattttacctgaatatggaaccctcgggctagaatgctcaccacggacaggtttggatttcaatgaaatgatctcattgacaatgaagaacaaagcatggatggtacatagacaatggttctttgacttacccctaccatggacatcaggagcttcagcagaaacaccaacttggaacaggaaagagcttcttgtgacatttaaaaatgcacatgcaaaaaagcaagaagtagttgttcttggatcacaagagggagcaatgcatacagcactgacaggagctacagagatccaaacctcaggaggcacaagtatctttgcggggcacttaaaatgtagactcaagatggacaaattggaactcaaggggatgagctatgcaatgtgcttgagtagctttgtgttgaagaaagaagtctccgaaacgcagcatgggacaatactcattaaggttgagtacaaaggggaagatgcaccctgcaagattcctttctccacggaggatggacaaggaaaagctctcaatggcagactgatcacagccaatccagtggtgaccaagaaggaggagcctgtcaacattgaggctgaacctccttttggagaaagtaacatagtaattggaattggagacaaagccctgaaaatcaactggtacaagaagggaagctcgattgggaagatgttcgaggccactgccagaggtgcaaggcgcatggccatcttgggagacacagcctgggactttggatcagtgggtggtgttttgaattcattagggaaaatggtccaccaaatatttgggagtgcttacacagccctatttggtggagtctcctggatgatgaaaattggaataggtgtcctcttaacctggatagggttgaactcaaaaaatacttctatgtcattttcatgcatcgcggccggcattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcgttgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccatgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 6 - protein, 3389 aa, synthetic construct
  source 1..3389: note = chimeric dengue serotype 2/3
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVIITVHTGDQHQVGNETQGVTAEITPQASTAEAILPEYGTLGLECSPRTGLDFNEMISLTMKNKAWMVHRQWFFDLPLPWTSGASAETPTWNRKELLVTFKNAHAKKQEVVVLGSQEGAMHTALTGATEIQTSGGTSIFAGHLKCRLKMDKLELKGMSYAMCLSSFVLKKEVSETQHGTILIKVEYKGEDAPCKIPFSTEDGQGKALNGRLITANPVVTKKEEPVNIEAEPPFGESNIVIGIGDKALKINWYKKGSSIGKMFEATARGARRMAILGDTAWDFGSVGGVLNSLGKMVHQIFGSAYTALFGGVSWMMKIGIGVLLTWIGLNSKNTSMSFSCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 7 - DNA, 10723 bp, synthetic construct
  source 1..10723: mol_type = other DNA; note = chimeric dengue seroytpe 2/4
agttgttagtctacgtggaccgacaaagacagattctttgagggagctaagctcaatgtagttctaacagttttttaattagagagcagatctctgatgaataaccaacggaaaaaggcgaaaaacacgcctttcaatatgctgaaacgcgagagaaaccgcgtgtcgactgtgcaacagctgacaaagagattctcacttggaatgctgcagggacgaggacctttaaaactgttcatggccctggtggcgttccttcgtttcctaacaatcccaccaacagcagggatattgaagagatggggaacaattaaaaaatcaaaagctattaatgttttgagagggttcaggaaagagattggaaggatgctgaacatcttgaataggagacgcagctctgcaggcatgatcattatgctgattccaacagtgatggcgttccatttaaccacgcgtgatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccttactggtcaataccgaacctgaagacattgattgctggtgcaatctcacgtctacctgggtcatgtatgggacatgcacccagagcggagaacggagacgagagaagcgctcagtagctttaacaccacattcaggaatgggattggaaacaagagctgagacatggatgtcatcggaaggggcttggaagcatgctcagagagtagagagctggatactcagaaacccaggattcgcgctcttggcaggatttatggcttatatgattgggcaaacaggaatccagcgaactgtcttctttgtcctaatgatgctggtcgccccatcctacggaatgcgatgcgtaggagtaggaaacagagactttgtggaaggagtctcaggtggagcatgggtcgatctggtgctagaacatggaggatgcgtcacaaccatggcccagggaaaaccaaccttggattttgaactgactaagacaacagccaaggaagtggctctgttaagaacctattgcattgaagcctcaatatcaaacataaccacggcaacaagatgtccaacgcaaggagagccttatctaaaagaggaacaagaccaacagtacatttgccggagagatgtggtagacagagggtggggcaatggctgtggcttgtttggaaaaggaggagttgtgacatgtgcgaagttttcatgttcggggaagataacaggcaatttggtccaaattgagaaccttgaatacacagtggttgtaacagtccacaatggagacacccatgcagtaggaaatgacacgtccaatcatggagttacagccacgataactcccaggtcaccatcggtggaagtcaaattgccggactatggagaactaacactcgattgtgaacccaggtctggaattgactttaatgagatgattctgatgaaaatgaaaaagaaaacatggcttgtgcataagcaatggtttttggatctacctctaccatggacagcaggagcagacacatcagaggttcactggaattacaaagagagaatggtgacatttaaggttcctcatgccaagagacaggatgtgacagtgctgggatctcaggaaggagccatgcattctgccctcgctggagccacagaagtggactccggtgatggaaatcacatgtttgcaggacatctcaagtgcaaagtccgtatggagaaattgagaatcaagggaatgtcatacacgatgtgttcaggaaagttctcaattgacaaagagatggcagaaacacagcatgggacaacagtggtgaaagtcaagtatgaaggtgctggagctccgtgtaaagtccccatagagataagagatgtgaacaaggaaaaagtggttgggcgtatcatctcatccacccctttggctgagaataccaacagtgtaaccaacatagagttagaacccccctttggggacagctacatagtgataggtgttggaaacagtgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtgcaccaggtttttggaagtgtgtatacaaccctgtttggaggagtctcatggatgattagaatcctaattgggttcctagtgttgtggattggcacgaactcaaggaacacttcaatggctatgacgtgcatagctgccggcattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagaggacatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagttaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcacagggaacatgtcctttagagacctgggaagagtgatggttatggtaggcgccactatgacgggtgacataggtatgggcgtgacttatcttgccctactagcagccttcaaagtcagaccaacttttgcagctggactactcttgagaaagctgacctccarggaattgatgatgactactataggaattgtactcctctcccagagcaccataccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacaatattggcagtggtgtccgtttccccactgttcttaacatcctcacagcaaaaaacagattggataccattagcattgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagcattgaagacaacccagagatcgaagatgacattttccgaaagagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgtgcacaccgggcgggagattgtggacctaatgtgtcatgccacatttaccatgaggctgctatcaccagttagagtgccaaactacaacctgattatcatggacgaagcccatttcacagatccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgctcgtggaattccggacatgaatgggtcacggattttaaagggaagactgtttggttcgttccaagtataaaagcaggaaatgatatagcagcttgcctgaggaaaaatggaaagaaagtgatacaactcagtaggaagacctttgattctgagtatgtcaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacctctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaaaacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggtaagagacgcactggacaacttagcagtgctgcacacggctgaggcaggtggaagggcgtacaaccatgctctcagtgaactgccggagaccctggagacattgcttttactgacacttctggctacagtcacgggagggatctttttattcttgatgagcgcaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctcggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagcyatagccaaccaagccacagtgttaatgggtctcgggaaaggatggccattgtcaaagatggacatcggagttccccttctcgccattggatgctactcacaagtcaaccccataactctcacagcagctcttttcttattggtagcacattatgccatcatagggccaggactccaagcaaaagcaaccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggctttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacacaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatgatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaatgattggacacaagtgcccttctgttcacaccatttccatgagttaatcatgaaagacggtcgcgtactcgttgttccctgtagaaaccaagatgaactgattggcagagcccgaatctcccaaggagcagggtggtctttgcgggagacggcctgtttggggaagtcttacgcccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtagaaagcaaaactaacatgaaacaaggctagaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 8 - protein, 3391 aa, synthetic construct
  VARIANT 1226: note = X -R or K
  source 1..3391: note = chimeric dengue seroytpe 2/4
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRSSAGMIIMLIPTVMAFHLTTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYGMRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFRKGSSIGKMFESTYRGAKRMAILGETAWDFGSVGGLFTSLGKAVHQVFGSVYTTLFGGVSWMIRILIGFLVLWIGTNSRNTSMAMTCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTGDIGMGVTYLALLAAFKVRPTFAAGLLLRKLTSXELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKVRDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW